(19) United States
(12) Patent Application Publication
(10) Pub. No.: US 2025/0078955 A1
(43) Pub. Date: Mar. 6, 2025

(54) DETECTING THE PRESENCE OF A TUMOR BASED ON METHYLATION STATUS OF CELL-FREE NUCLEIC ACID MOLECULES

(71) Applicant: GUARDANT HEALTH, INC., Palo Alto, CA (US)

(72) Inventors: Sai CHEN, San Diego, CA (US); Katie Julia QUINN, Redwood City, CA (US); Tingting JIANG, San Diego, CA (US); Jun ZHAO, San Diego, CA (US)

(21) Appl. No.: 18/628,053

(22) Filed: Apr. 5, 2024

Related U.S. Application Data
(60) Provisional application No. 63/494,984, filed on Apr. 7, 2023.

(51) Int. Cl. G16B 20/20; G16B 40/20; G16H 50/20

(52) U.S. Cl. CPC G16B 20/20; G16B 40/20; G16H 50/20

(57) ABSTRACT

In implementations described herein, methylation information is determined with respect to classification regions of a reference genome that are related to the presence of a tumor in a subject. The methylation information can be analyzed using a number of computational techniques to provide metrics related to the presence or absence of a tumor in a given subject, including a determination of tumor fraction.

### CROSS REFERENCE TO RELATED APPLICATIONS

This application claims the benefit of U.S. provisional patent application No. 63/494,984 filed Apr. 7, 2023, which is incorporated by reference herein in its entirety.

### BACKGROUND

Cancer is a major cause of disease worldwide. Each year, tens of millions of people are diagnosed with cancer around the world, and more than half eventually die from it. In many countries, cancer ranks the second most common cause of death following cardiovascular diseases. Early detection is associated with improved outcomes for many cancers.

Cancer can be caused by the accumulation of genetics variations within an individual's normal cells, at least some of which result in improperly regulated cell division. Such variations commonly include copy number variations (CNVs), single nucleotide variations (SNVs), gene fusions, insertions and/or deletions (indels), epigenetic variations including 5-methylation of cytosine (5-methylcytosine) and association of DNA with chromatin and transcription factors.

Cancers are often detected by biopsies of tumors followed by analysis of cells, markers or DNA extracted from cells. But more recently it has been proposed that cancers can also be detected from cell-free nucleic acids in body fluids, such as blood or urine. Such tests have the advantage that they are noninvasive and can be performed without identifying suspected cancer cells in biopsy. However, such tests are complicated by the fact that the amount of nucleic acids in body fluids is very low and what nucleic acids are present are heterogeneous in form (e.g., RNA and DNA, single-stranded and double-stranded, and various states of post-replication modification and association with proteins, such as histones).

Thus, there is a need for improved systems and methods for improved cancer detection using liquid biopsy assays. Therefore, it is an object of the disclosure to provide computer-implemented systems and methods that have improved capability to classify a sample as containing tumor-derived DNA with heightened sensitivity.

Circulating tumor DNA (ctDNA) level and change in ctDNA level on-treatment are promising tools for predicting patient prognosis and response to therapy.

Existing methods commonly use variant allele frequency (VAF) of somatic mutations to quantify circulating tumor fraction (cTF). Their performance can be limited by the number of detectable somatic mutations and the associated limit of detection (LoD), as well as interference from copy number variation and non-tumor alterations, such as clonal hematopoiesis (CH). Moreover, previous studies also show that 30-50% patients with stage I-III cancer, and 15-20% patients with stage IV cancer, lack detectable somatic mutations1.

Combined genomic and/or epigenomic detection assay described herein, provides a unique combined genomic and epigenomic molecular profile revealing unseen insights distinctive to each sample from a single blood draw. Described herein are methods and compositions for determining measurement of tumor fraction using methylation, including detection using a combined genomic and/or epigenomic detection assay described herein epigenomic panel which allows for near genome-wide methylation detection.

### BRIEF DESCRIPTION OF THE DRAWINGS

FIG. 1 is a diagrammatic representation of an example environment 100 that identifies nucleic acids that correspond to classification regions of a reference sequence, where the classification regions have at least a threshold number of CpGs

FIG. 2 is a diagrammatic representation of an example architecture to determine tumor metrics based on one or more models that analyze methylation status of cell free nucleic acid molecules, according to one or more implementations.

FIG. 3 is a diagrammatic representation of an example architecture to train one or more machine learning models to determine cancer metrics based on methylation status of cell-free nucleic acid molecules, according to one or more implementations.

FIG. 4 is a flow diagram of an example process to determine tumor metrics related to levels of methylation of classification regions of a reference sequence, according to one or more implementations.

FIG. 5 is a block diagram illustrating components of a machine, in the form of a computer system, that may read and execute instructions from one or more machine-readable media to perform any one or more methodologies described herein, in accordance with one or more example implementations.

FIG. 6 is block diagram illustrating a representative software architecture that may be used in conjunction with one or more hardware architectures described herein, in accordance with one or more example implementations.

FIGS. 7A, 7B, and 7C are graphical representations showing promoter-methylation calls in training and test samples among 88 TSG+HRD genes.

FIG. 8A is a graphical representation showing cancer-prediction scores in cancer-free samples that have >1 call. FIG. 8B is a table showing genes that called most often for promoter methylation in cancer-free donors. FIG. 8C is a table showing in-silico LoD estimates in selected genes from cell line KM12.

FIG. 9 is a graphical representation showing prevalence of promoter methylation in different types of cancer of TSG and HRD genes, our test dataset (total N=559), and in TCGA public data (total N=2,380). Limited to genes with promoter methylation in TCGA.

FIG. 10A is a graphical representation showing MLH1 promoter methylation in cancer-free donors and CRC patients (MSI-H and MSS). FIG. 10B is a table showing calls of MLH1 promoter methylation and BRAF-V600E in CRC patients.

FIG. 11 is a table showing an overview of the training and the test datasets for Example 5.

FIG. 12A is a graph graphical representation showing model performance for the prediction of CRC/cancer-free status in the training set. Shadows indicate variations in iterations. FIG. 12B is a table showing performance of cancer prediction models on the independent test dataset.

FIG. 13A is a table showing CV of TF estimates from genomic calls and methylation in the in-vitro dataset. FIG. 13B is a graphical representation showing TF model performance (black lines for diagonals) in the training set of CRC and cancer-free samples (cross-validation) FIG. 13C is a graphical representation showing the in-silico dataset for lower truth TFs.

FIG. 14 is a graphical representation showing distribution of predicted TF for CRC patients in the training set with and without driver mutations.

FIG. 15A is a graphical representation showing positivity rates in individual for lung cancer detection in stage I/II patients and in stage III/IV. patients.

FIG. 15B is a graphical representation showing positivity rates in individuals for multi-cancer detection (bladder, gastric, ovarian, pancreatic, and liver) in stage I/II patients and in stage III/IV patients.

FIG. 16A is a graphical representation showing positivity rates in individuals for multi-cancer detection (bladder, gastric, ovarian, pancreatic, and liver) in stage I patients, stage II patients, stage III patients, and stage IV patients.

FIG. 17 is a graphical representation of epigenomic MAF in relation to target MAF for colorectal cancer, lung cancer, and breast cancer.

FIG. 18 is a table showing that the quantitative precision of epigenomics cTF is capable of reaching an LoQ of less than 0.1% in CRC, lung and breast clinical samples.

FIG. 19A is a graphical representation showing that the somatic mutation based cTF is robust for replicates within the same cTF levels, particularly at cTF levels of 0.5% or higher.

FIG. 19B is a graphical representation showing that the epigenomic cTF can maintain a 100% evaluation rate and has a LoQ down to 0.1% cTF.

FIG. 20A is a graphical representation of methylation signals and somatic mutations for a first replicate of clinical titrations.

FIG. 20B is a graphical representation of methylation signals and somatic mutations for a second replicate of clinical titrations.

FIG. 21 is a table indicating ctDNA level changes for the first replicate and the second replicate calculated using a genomic-only method and a methylation method.

FIG. 22 is a graphical representation of epigenomic vs genomic cTF on clinical samples (one point for one sample).

FIG. 23 is a graphical representation of the epiMAF distribution in early and late-stage cancer patients for breast cancer, colorectal cancer, lung cancer, and a group of other cancers.

FIG. 24 is a graphical representation of CpG counts in different partitions including hyper, hypo and residual partitions.

FIG. 25 is a graphic representation of exemplary region scores, including when using normalization.

FIG. 26 is a representation of serial sample % TF using relative change of patient-specific DMRs. Serial samples' % TF could be calculated using the relative change from a previous sample. Can identify patient-specific DMRs that track tumor dynamics. Then these can track TF to low levels with high specificity (since we know they are tumor).

For example: Two longitudinal samples from a BRAF+ CRC patient: Methylation detects tumor at both timepoints. % TF from single-sample method and relative change agree, but relative change can use more DMRs, which could enable quantification down to lower levels.

FIG. 27 is representation of Epi MAF by “normalizing” molecules in each peak first Ctrl molecules were smoothed before using to normalize other regions.

FIG. 28 is a representation of pre-select regions from internal training data

Exclude regions with too frequent signals in normal. In the current single-sample setting, it can be challenging to distinguish normal/cancer signals. Select high-prevalence cancer-specific regions A trade-off between selecting low-prevalence regions (more signals) vs additional noise

FIG. 29 is a representation of further select top regions to estimate epi MAF From prevalence analysis—not all regions are methylated in all samples. Better to define the possibly methylated regions in each sample. Titrations (in silico and experimental) indicate an LoD of ˜0.1% for 30 ng clinical samples. epiTF is reported when methylation is detected. Limit of Quantification (LoQ), the TF where variation <=defined threshold, focus on >=0.1%. A representation of estimate sample TF % from sample's top regions can be made using a decision tree: Among the selected regions select the sample's 100 regions with highest methylation “VAF” % TF=Average methylation “VAF” of these regions. In some instances, one can take 5%-95% quantile to avoid outliers. If fewer than 100 regions have any molecules then fill with zeroes.

FIG. 30 is a representation of performance in a cohort: % TF is consistent with both genomic % TF and paired-sample “informed” method 48 patients with pre and post surgery runs on methylation detection platform. Results from current method is consistent with our previous method which uses the paired-sample info to estimate TF

FIG. 31 is a representation of an example of benefit of Paired sample relative analysis in a cohort. Observation: In a BRAF V600E driver cohort, a few samples (˜8/408) are BRAF+ but Epi-neg. The BRAF is usually <0.1%. Approach: For serial samples, look for the epi signal from high-TF sample in the low-TF sample (is it still there?), and also look for what can interfere with Epi Tumor detection in the low-TF sample. Result: In 1 patient with 3 samples: In the 3rd sample, the epi tumor signal is reduced to the norm_mol level of the background (the background is at the same level across the 3 samples). But the tumor signal is still obvious and quantifiable by using the regions that had been high in the previous samples.

FIG. 32. Accuracy, Limit of Quantification (LoQ) and Coefficient of Variation (CV) of methyl cTF in replicates of clinical titrations. Clinical titrations=clinical cancer samples experimentally titrated into a cancer-free donor sample at known fractions

FIG. 33. Estimated cTF ratio between replicates. In somatic-mutation based methods, 15-20% stage iv patients have no detectable signals (“ctDNA low”). With methylation, there are still >1,000 regions with detectable signals at cTF as low as 0.1%.

FIG. 34. Methylation signals and somatic mutations in two pairs of replicates of clinical titrations (0.5% vs 0.3% cTF).

FIG. 35. Methyl vs genomic cTF on clinical samples (one point for one clinical sample). “Driver”=genomic maxMAF from predefined “driver” genes (more accurate representation of true cTF). Color of left side bars show methyl cTF of samples that do not have detectable somatic mutations

FIG. 36. The methyl cTF distribution in early and late stage cancer patients

### SUMMARY OF THE INVENTION

Described herein is a method including obtaining training sequence data including training sequencing reads derived from a plurality of samples of a plurality of subjects, individual training sequencing reads including a nucleotide sequence corresponding to a fragment of a nucleic acid included in a sample of the plurality of samples and an amount of methylated cytosines included in regions of the nucleotide sequence having cytosine-guanine content, analyzing the training sequencing reads to determine a first quantitative measure derived from the training sequencing reads that corresponds to individual classification regions of a plurality of classification regions, at least a portion of the individual classification regions of the plurality of classification regions corresponding to genomic regions of a reference genome that have an amount of methylated cytosines in subjects in which cancer is detected, analyzing the training sequencing reads to determine a second quantitative measure derived from the training sequencing reads that correspond to a plurality of control regions, individual control regions of the plurality of control regions corresponding to additional genomic regions of the reference genome that have an amount of methylated cytosines in which cancer is not detected, determining a metric for the individual classification regions of the plurality of classification regions based on the first quantitative measure for the individual classification regions and the second quantitative measure for the plurality of control regions, generating training data that includes the metric for the individual classification regions of the plurality of classification regions for the training sequence reads from samples of training subjects,
implementing one or more machine learning algorithms to generate a model, the model including weights for individual classification regions of the plurality of classification regions and at least a portion of the weights of the individual classification regions being different from one another. In other embodiments, the method includes obtaining testing sequence data from an additional subject that is not included in the plurality of subjects, the testing sequence data including testing sequencing reads derived from a sample of the additional subject, individual testing sequencing reads including a nucleotide sequence corresponding to a fragment of a nucleic acid included in the additional sample and individual testing sequencing reads corresponding to molecules having an amount of methylated cytosines included in regions of the nucleotide sequence having cytosine-guanine content, and determining, using the model and the additional sequence data, a measurement of tumor fraction in the additional subject. from one another.

In other embodiments, the method includes selecting a sub-set of the plurality of classification regions. from one another. In other embodiments, the sub-set of the plurality of classification regions comprise one or more cancer-specific regions. from one another. In other embodiments, metric for the individual classification regions of the plurality of classification regions based on the first quantitative measure for the individual classification regions and the second quantitative measure for the plurality of control regions includes a sub-set of the plurality of classification regions. from one another.

In other embodiments, the method includes analyzing the testing sequencing reads to determine a first quantitative measure derived from the testing sequencing reads that correspond to the individual classification regions of the plurality of classification regions, analyzing the testing sequencing reads to determine a second quantitative measure derived from the testing sequencing reads that correspond to the individual control regions of the plurality of control regions, normalizing the second quantitative measure based on the corresponding individual control regions of the plurality of control regions, determining the metric for the individual classification regions based on the first quantitative measure for the individual classification regions and the normalized second quantitative measure for the plurality of control regions, and applying a machine learning algorithm to the metrics for the individual classification regions to determine a measurement of tumor fraction in the additional subject. from one another.

In other embodiments, the one or more machine learning algorithms include one or more classification algorithms. from one another. In other embodiments, the one or more machine learning algorithms include one or more regression algorithms. In other embodiments, the method includes applying a machine learning algorithm to the metrics for the individual classification regions to determine a measurement of tumor fraction in the additional subject includes selecting a sub-set of the plurality of classification regions. In other embodiments, the training data includes the individual measures of tumor fraction for the individual samples of the plurality of samples, and the model is generated based on the individual measures of tumor fraction for the individual samples of the plurality of samples. In other embodiments, the metric for the individual classification regions is determined based on a scaling factor and/or an error correction factor. In other embodiments, the plurality of classification regions individually correspond to genomic regions in which a methylation rate of cytosines in the genomic regions of nucleic acids derived from cells obtained from subjects in which cancer is present is different from a methylation rate of cytosines in the genomic regions of nucleic acids derived from cells obtained from subjects in which cancer is not present. In other embodiments, the plurality of classification regions correspond to a first plurality of classification regions for a first cancer type and the model can be generated for a second cancer type based on a second plurality of classification regions that are different from the first plurality of classification regions.

Further described herein is a method including obtaining sequencing reads derived from a sample obtained from a subject, individual sequencing reads including a nucleotide sequence corresponding to a fragment of a nucleic acid included in the sample and corresponding to molecules having a threshold amount of methylated cytosines included in regions of the nucleotide sequence having cytosine-guanine content, determining, by the computing system, a first quantitative measure derived from the sequencing reads that corresponds to individual classification regions of a plurality of classification regions, at least a portion of the individual classification regions of the plurality of classification regions corresponding to genomic regions of a reference genome with amount of methylated cytosines in subjects in which cancer is detected, analyzing, by the computing system, the sequencing reads to determine a second quantitative measure derived from the sequencing reads that correspond to a plurality of control regions, individual control regions of the plurality of control regions corresponding to additional genomic regions of the reference genome that have cytosine-guanine content and an amount of methylated cytosines in additional subjects in which cancer is not detected, determining, by the computing system, a plurality of metrics with individual metrics of the plurality of metrics corresponding to individual classification regions of the plurality of classification regions based on the first quantitative measure for the individual classification regions and the second quantitative measure for the plurality of control regions, and determining a measurement of tumor fraction in the additional subject.

In other embodiments, the method includes selecting a sub-set of the plurality of classification regions. In other embodiments, the sub-set of the plurality of classification regions comprise one or more cancer-specific regions. In other embodiments, the metric for the individual classification regions of the plurality of classification regions based on the first quantitative measure for the individual classification regions and the second quantitative measure for the plurality of control regions includes a sub-set of the plurality of classification regions. In other embodiments, the method includes determining an order of the values of the plurality of metrics, and determining a subset of classification regions from among the plurality of classification regions based on the order, wherein a portion of the plurality of metrics that correspond to the subset of the classification regions is used to determine a measurement of tumor fraction in the additional subject. In other embodiments, the method includes determining a measurement of tumor fraction in the additional subject includes applying a scaling factor. In other embodiments, the determined measurement of tumor fraction corresponds to an indication of cancer status in the subject. In other embodiments, the method includes determining a measurement of tumor fraction in the subject includes, applying a model generated from training data.

In other embodiments, the model generated from training data includes: obtaining training sequence data including training sequencing reads derived from a plurality of samples of a plurality of subjects, individual training sequencing reads including a nucleotide sequence corresponding to a fragment of a nucleic acid included in a sample of the plurality of samples and an amount of methylated cytosines included in regions of the nucleotide sequence having cytosine-guanine content, analyzing the training sequencing reads to determine a first quantitative measure derived from the training sequencing reads that corresponds to individual classification regions of a plurality of classification regions, at least a portion of the individual classification regions of the plurality of classification regions corresponding to genomic regions of a reference genome that have a threshold amount of methylated cytosines in subjects in which cancer is detected and that have at least the threshold cytosine-guanine content, analyzing the training sequencing reads to determine a second quantitative measure derived from the training sequencing reads that correspond to a plurality of control regions, individual control regions of the plurality of control regions corresponding to additional genomic regions of the reference genome that have at least the threshold cytosine-guanine content and that have a threshold amount of methylated cytosines in which cancer is not detected, determining a metric for the individual classification regions of the plurality of classification regions based on the first quantitative measure for the individual classification regions and the second quantitative measure for the plurality of control regions, generating training data that includes the metric for the individual classification regions of the plurality of classification regions for the training sequence reads from samples of training subjects, implementing one or more machine learning algorithms to generate the model.

Also described herein is method including: obtaining testing sequence data from a subject, the testing sequence data including testing sequencing reads derived from a sample of the subject, individual testing sequencing reads including a nucleotide sequence corresponding to a fragment of a nucleic acid included in the additional sample and individual testing sequencing reads corresponding to molecules having an amount of methylated cytosines included in regions of the nucleotide sequence, analyzing the testing sequencing reads to determine a first quantitative measure derived from the testing sequencing reads that correspond to individual classification regions of a plurality of classification regions at least a portion of the individual classification regions of the plurality of classification regions corresponding to genomic regions of a reference genome that have an amount of methylated cytosines in subjects in which cancer is detected, analyzing the testing sequencing reads to determine a second quantitative measure derived from the testing sequencing reads that correspond to individual control regions a plurality of control regions, individual control regions of the plurality of control regions corresponding to additional genomic regions of the reference genome that have an amount of methylated cytosines in additional subjects in which cancer is not detected,
determining a metric for the individual classification regions based on the first quantitative measure for the individual classification regions and the second quantitative measure for the plurality of control regions, and generating training data that includes the metric for the individual classification regions of the plurality of classification regions for the training sequence reads from samples of training subjects,
implementing one or more machine learning algorithms to generate a model, the model including weights for individual classification regions of the plurality of classification regions and at least a portion of the weights of the individual classification regions being different from one another to determine a measurement of tumor fraction in the subject. In other embodiments, the method includes obtaining training sequence data including training sequencing reads derived from a plurality of samples of a plurality of training subjects, individual training sequencing reads including a nucleotide sequence corresponding to a fragment of a nucleic acid included in a sample of the plurality of samples and individual training sequencing reads corresponding to molecules having a threshold amount of methylated cytosines included in regions of the nucleotide sequence having at least a threshold cytosine-guanine content, analyzing the training sequencing reads to determine an additional first quantitative measure derived from the training sequencing reads that corresponds to individual classification regions of the plurality of classification regions, analyzing the training sequencing reads to determine an additional second quantitative measure derived from the training sequencing reads that correspond to a plurality of control regions, determining an additional metric for the individual classification regions of the plurality of classification regions based on the additional first quantitative measure for the individual classification regions and the additional second quantitative measure for the plurality of control regions, generating training data that includes the additional metric for the individual classification regions of the plurality of classification regions for the training sequence reads from samples of the plurality of training subjects, implementing using the training data, one or more machine learning algorithms to generate the model to determine the indications of cancer status in subjects based on amounts of methylated cytosines in at least a portion of the plurality of classification regions. In other embodiments, the one or more machine learning algorithms include one or more classification algorithms. In other embodiments, the one or more machine learning algorithms include one or more regression algorithms, and the indication corresponds to an estimate of tumor fraction of the sample. In other embodiments, the method includes the training sequencing reads comprise a first portion of the training sequence data and additional training sequencing reads comprise a second portion of the training sequence data, wherein the additional training sequencing reads are different from the training sequencing reads, and the method including: analyzing at least one of the first portion of the training sequence data or the second portion of the training sequence data to determine an individual frequency of a plurality of variants present in an individual sample of the plurality of samples, determining for the individual sample, a variant of the plurality of variants having a maximum frequency that corresponds to the individual frequency having a greatest value among individual frequencies derived from an individual sample, and determining individual measures of tumor fraction for an individual sample based on the greatest value of the individual frequencies derived from the individual sample. In other embodiments, the training data includes the individual measures of tumor fraction for the individual samples of the plurality of samples, and the model is generated based on the individual measures of tumor fraction for the individual samples of the plurality of samples. In other embodiments, the sample of the subject and the plurality of samples of the plurality of training subjects include cell free nucleic acids.

Further described herein is a method including: obtaining sequencing reads derived from one or more samples obtained from a subject, individual sequencing reads including a nucleotide sequence corresponding to a fragment of a nucleic acid included in the sample and corresponding to molecules having a threshold amount of methylated cytosines included in regions of the nucleotide sequence having at least a threshold cytosine-guanine content, determining a first quantitative measure derived from the sequencing reads that corresponds to individual classification regions of a plurality of classification regions, at least a portion of the individual classification regions of the plurality of classification regions corresponding to genomic regions of a reference genome that an amount of methylated cytosines in subjects in which cancer is detected and that have at least the threshold cytosine-guanine content, analyzing the sequencing reads to determine a second quantitative measure derived from the sequencing reads that correspond to a plurality of control regions, individual control regions of the plurality of control regions corresponding to additional genomic regions of the reference genome that have at least the threshold cytosine-guanine content and that have an amount of methylated cytosines in additional subjects in which cancer is not detected, determining a plurality of metrics with individual metrics of the plurality of metrics corresponding to individual classification regions of the plurality of classification regions based on the first quantitative measure for the individual classification regions and the second quantitative measure for the plurality of control regions, and determining an indication of cancer status in the subject based on at least a portion of the plurality of metrics. In other embodiments, the method includes selecting a sub-set of the plurality of classification regions. In other embodiments, the sub-set of the plurality of classification regions comprise one or more cancer-specific regions. In other embodiments, the metric for the individual classification regions of the plurality of classification regions based on the first quantitative measure for the individual classification regions and the second quantitative measure for the plurality of control regions includes a sub-set of the plurality of classification regions. In other embodiments, the method includes at least two samples obtained from a subject In other embodiments, the method includes determining a metric for the individual classification regions of the plurality of classification regions based on the first quantitative measure for the individual classification regions and the second quantitative measure for the plurality of control regions comprises: selecting a sub-set of the plurality of classification regions based on a regression algorithm based on the first quantitative measure for the individual classification regions and the second quantitative measure for the plurality of control regions. In other embodiments, the first quantitative measure is normalized based on second quantitative measure. In other embodiments, the plurality of samples and the additional sample include cell free nucleic acids. In other embodiments, the method includes combining a plurality of nucleic acids derived from at least one of blood or tissue of a subject with a solution including an amount of methyl binding domain (MBD) proteins to produce a nucleic acid-MBD protein solution, and performing a plurality of washes of the nucleic acid-MBD protein solution with a salt solution to produce a number of nucleic acid fractions, individual nucleic acid fractions having a threshold number of methylated cytosines in regions of the plurality of nucleic acids having at least the threshold cytosine-guanine content. In other embodiments, the method includes a wash of the plurality of washes is performed with a solution having a concentration of sodium chloride (NaCl) and produces a nucleic acid fraction of the number of nucleic acid fractions having a range of binding strengths to MBD proteins. In other embodiments, the method includes determining that a first nucleic acid fraction is associated with a first partition of a plurality of partitions of nucleic acids, the first partition corresponding to a first range of binding strengths to MBD proteins, attaching a first molecular barcode to nucleic acids of the first nucleic acid fraction, the first molecular barcode being included in a first set of molecular barcodes associated with the first partition, determining that a second nucleic acid fraction is associated with a second partition of the plurality of partitions of nucleic acids, the second partition corresponding to a second range of binding energies to MBD proteins different from the first range of binding strengths to MBD proteins, and attaching a second molecular barcode to nucleic acids of the second nucleic acid fraction, the second molecular barcode being included in a second set of molecular barcodes associated with the second partition. In other embodiments, the method includes combining at least a portion of the number of nucleic acid fractions with an amount of restriction enzyme that cleaves molecules with one or more unmethylated cytosines to produce at least a portion of the plurality of samples used to produce the sequencing reads, wherein the threshold amount of methylated cytosines corresponds to a minimum frequency of methylated cytosines within a region having at least the threshold cytosine-guanine content. In other embodiments, the method includes combining at least a portion of the number of nucleic acid fractions with an amount of a restriction enzyme that cleaves molecules with one or more methylated cytosines to produce at least a portion of the plurality of samples used to produce the sequencing reads, wherein the threshold amount of unmethylated cytosines corresponds to a maximum frequency of methylated cytosines that are not cleaved within a region having at least the threshold cytosine-guanine content. In other embodiments, the method includes a limit of detection for the model to determine tumor fraction of samples is no greater than 0.05%. 0.05%.

In one or more aspects, a method includes obtaining, by a computing system having one or more hardware processors and memory, training sequence data including training sequencing reads derived from a plurality of samples of a plurality of subjects, individual training sequencing reads including a nucleotide sequence corresponding to a fragment of a nucleic acid included in a sample of the plurality of samples and individual training sequencing reads corresponding to molecules having a threshold amount of methylated cytosines included in regions of the nucleotide sequence having at least a threshold cytosine-guanine content. The method also includes analyzing, by the computing system, the training sequencing reads to determine a first quantitative measure derived from the training sequencing reads that corresponds to individual classification regions of a plurality of classification regions, at least a portion of the individual classification regions of the plurality of classification regions corresponding to genomic regions of a reference genome that have the threshold amount of methylated cytosines in subjects in which cancer is detected and that have at least the threshold cytosine-guanine content. The method also includes analyzing, by the computing system, the training sequencing reads to determine a second quantitative measure derived from the training sequencing reads that correspond to a plurality of control regions, individual control regions of the plurality of control regions corresponding to additional genomic regions of the reference genome that have at least the threshold cytosine-guanine content and that have at least the threshold amount of methylated cytosines in subjects in which cancer is detected and in additional subjects in which cancer is not detected. The method also includes determining, by the computing system, a metric for the individual classification regions of the plurality of classification regions based on the first quantitative measure for the individual classification regions and the second quantitative measure for the plurality of control regions. The method also includes generating, by the computing device, training data that includes the metric for the individual classification regions of the plurality of classification regions for the training sequence reads from samples of training subjects. The method also includes implementing, by the computing system and using the training data, one or more machine learning algorithms to generate a model to determine an indication of cancer status in subjects based on amounts of methylated cytosines in at least a portion of the plurality of classification regions, the model including weights for individual classification regions of the plurality of classification regions and at least a portion of the weights of the individual classification regions being different from one another.

In one or more aspects, the method includes obtaining, by the computing system, testing sequence data from an additional subject that is not included in the plurality of subjects, the testing sequence data including testing sequencing reads derived from a sample of the additional subject, individual testing sequencing reads including a nucleotide sequence corresponding to a fragment of a nucleic acid included in the additional sample and individual testing sequencing reads corresponding to molecules having at least the threshold amount of methylated cytosines included in regions of the nucleotide sequence having at least the threshold cytosine-guanine content, and determining, using the model and the additional sequence data, the indication of cancer status in the additional subject.

In one or more aspects, the method includes analyzing, by the computing system, the testing sequencing reads to determine a first quantitative measure derived from the testing sequencing reads that correspond to the individual classification regions of the plurality of classification regions, analyzing, by the computing system, the testing sequencing reads to determine a second quantitative measure derived from the testing sequencing reads that correspond to the individual control regions the plurality of control regions, determining, by the computing system, the metric for the individual classification regions based on the first quantitative measure for the individual classification regions and the second quantitative measure for the plurality of control regions, and generating, by the computing system, an input vector that includes the metrics for the individual classification regions, where the model uses the input vector to determine the indication of cancer status in the additional subject. In one or more aspects, the one or more machine learning algorithms include one or more classification algorithms and the indication of cancer status corresponds to a probability of cancer status in the additional subject. In one or more aspects, the one or more machine learning algorithms include one or more regression algorithms and the indicator corresponds to an estimate of tumor fraction of the additional sample.

In one or more aspects, the training sequencing reads comprise a first portion of the training sequence data and additional training sequencing reads comprise a second portion of the training sequence data, where the additional training sequencing reads are different from the training sequencing reads and the method includes analyzing, by the computing system, at least one of the first portion of the training sequence data or the second portion of the training sequence data to determine an individual frequency of a plurality of variants present in an individual sample of the plurality of samples, determining, by the computing system and for the individual samples, a variant of the plurality of variants having a maximum frequency that corresponds to the individual frequency having a greatest value among individual frequencies derived from an individual sample, and determining, by the computing system, individual measures of tumor fraction for an individual sample based on the greatest value of the individual frequencies derived from the individual sample. In one or more aspects, the training data includes the individual measures of tumor fraction for the individual samples of the plurality of samples and the model is generated based on the individual measures of tumor fraction for the individual samples of the plurality of samples. In one or more aspects, the metric for the individual classification regions is determined based on a scaling factor and an error correction factor. In one or more aspects, the plurality of classification regions individually correspond to genomic regions in which a methylation rate of the genomic regions in nucleic acids derived from cells obtained from subjects in which cancer is present is different from a methylation rate of the genomic regions in nucleic acids derived from cells obtained from subjects in which cancer is not present.

In one or more aspects, the plurality of classification regions correspond to a first plurality of classification regions for a first cancer type and the model can be generated for a second cancer type based on a second plurality of classification regions that are different from the first plurality of classification regions. In one or more aspects, the plurality of samples and the additional sample include cell free nucleic acids. In one or more aspects, the method includes performing, by the computing system, a training process using the training data to generate the model, where the training process includes determining, by the computing system, one or more additional weights of individual samples included in the training data based on the indication of cancer for the individual samples being within a threshold confidence level. In one or more aspects, the indication of cancer for an individual sample is outside of the threshold confidence level and the method includes applying, by the computing system, a penalty to a weight of the individual sample during the training process. In one or more aspects, the method includes performing, by the computing system and using the one or more machine learning algorithms, one or more first iterations of the training process for the model using a portion of the training data, and generating, by the computing system, first output data for the model based on the one or more first iterations of the training process, the first output data corresponding to one or more first additional indications of cancer status in first individual subjects of the plurality of subjects, the first individual subjects corresponding to the portion of the training data.

In one or more aspects, the method includes combining, by the computing system, the first output data and the training data to produce additional training data, performing, by the computing system, one or more second iterations of the training process for the model using a portion of the additional training data, and generating, by the computing system, second output data for the model based on the one or more second iterations of the training process, the second output data indicating one or more second additional indications of cancer status in second individual subjects of the plurality of subjects, the second individual subjects corresponding to the portion of the additional training data. In one or more aspects, the weights for the individual classification regions of the plurality of classification regions are determined based on the first output data and the second output data. In one or more aspects, the method includes determining, by the computing system, that a number of indications of cancer status that were determined during one or more iterations of the training process are at least a threshold value for one or more samples included in the training data, and determining, by the computing system, that modifications to one or more weights of the model are not modified or are modified by a minimal amount. In one or more aspects, the method includes determining, by the computing system, that an additional number of indications of cancer status that were determined during the one or more iterations of the training process are less than the threshold value for one or more additional samples included in the training data, and determining, by the computing system, that modifications to one or more additional weights of the model are modified by more than the minimal amount.

In one or more aspects, the method includes combining a plurality of nucleic acids derived from at least one of blood or tissue of a subject with a solution including an amount of methyl binding domain (MBD) proteins to produce a nucleic acid-MBD protein solution, and performing a plurality of washes of the nucleic acid-MBD protein solution with a salt solution to produce a number of nucleic acid fractions, individual nucleic acid fractions having a threshold number of methylated cytosines in regions of the plurality of nucleic acids having at least the threshold cytosine-guanine content.

In one or more aspects, a wash of the plurality of washes is performed with a solution having a concentration of sodium chloride (NaCl) and produces a nucleic acid fraction of the number of nucleic acid fractions having a range of binding strengths to MBD proteins.

In one or more aspects, the method includes determining that a first nucleic acid fraction is associated with a first partition of a plurality of partitions of nucleic acids, the first partition corresponding to a first range of binding strengths to MBD proteins, attaching a first molecular barcode to nucleic acids of the first nucleic acid fraction, the first molecular barcode being included in a first set of molecular barcodes associated with the first partition, determining that a second nucleic acid fraction is associated with a second partition of the plurality of partitions of nucleic acids, the second partition corresponding to a second range of binding strengths to MBD proteins different from the first range of binding strengths to MBD proteins, and attaching a second molecular barcode to nucleic acids of the second nucleic acid fraction, the second molecular barcode being included in a second set of molecular barcodes associated with the second partition.

In one or more aspects, the method includes combining at least a portion of the number of nucleic acid fractions with an amount of one or more methylation sensitive restriction enzymes that cleave molecules with one or more unmethylated cytosines to produce at least a portion of the plurality of samples used to produce the sequencing reads. In one or more aspects, the method includes combining at least a portion of the number of nucleic acid fractions with an amount of one or more methylation dependent restriction enzymes that cleaves molecules with one or more methylated cytosines to produce at least a portion of the plurality of samples used to produce the sequencing reads. In one or more aspects, a limit of detection for the model to determine tumor fraction of samples is no greater than 0.05%.

In one or more aspects, a computing system includes: one or more hardware processors, and one or more non-transitory computer-readable storage media including computer-readable instructions that, when executed by the one or more hardware processors, cause the one or more hardware processors to perform operations including: obtaining training sequence data including training sequencing reads derived from a plurality of samples of a plurality of subjects, individual training sequencing reads including a nucleotide sequence corresponding to a fragment of a nucleic acid included in a sample of the plurality of samples and individual training sequencing reads corresponding to molecules having a threshold amount of methylated cytosines included in regions of the nucleotide sequence having at least a threshold cytosine-guanine content. The operations also include analyzing the training sequencing reads to determine a first quantitative measure derived from the training sequencing reads that corresponds to individual classification regions of a plurality of classification regions, at least a portion of the individual classification regions of the plurality of classification regions corresponding to genomic regions of a reference genome that have the threshold amount of methylated cytosines in subjects in which cancer is detected and that have at least the threshold cytosine-guanine content. The operations also include analyzing the training sequencing reads to determine a second quantitative measure derived from the training sequencing reads that correspond to a plurality of control regions, individual control regions of the plurality of control regions corresponding to additional genomic regions of the reference genome that have at least the threshold cytosine-guanine content and that have at least the threshold amount of methylated cytosines in subjects in which cancer is detected and in additional subjects in which cancer is not detected. The operations also include determining a metric for the individual classification regions of the plurality of classification regions based on the first quantitative measure for the individual classification regions and the second quantitative measure for the plurality of control regions. The operations also include generating training data that includes the metric for the individual classification regions of the plurality of classification regions for the training sequence reads from samples of training subjects. The operations also include implementing, using the training data, one or more machine learning algorithms to generate a model to determine an indication of cancer status in subjects based on amounts of methylated cytosines in at least a portion of the plurality of classification regions, the model including weights for individual classification regions of the plurality of classification regions and at least a portion of the weights of the individual classification regions being different from one another.

In one or more aspects, one or more computer-readable storage media comprise computer-readable instructions that, when executed by one or more processors of a computing system, cause the computing system to perform operations including: obtaining training sequence data including training sequencing reads derived from a plurality of samples of a plurality of subjects, individual training sequencing reads including a nucleotide sequence corresponding to a fragment of a nucleic acid included in a sample of the plurality of samples and individual training sequencing reads corresponding to molecules having a threshold amount of methylated cytosines included in regions of the nucleotide sequence having at least a threshold cytosine-guanine content. The operations also include analyzing the training sequencing reads to determine a first quantitative measure derived from the training sequencing reads that corresponds to individual classification regions of a plurality of classification regions, at least a portion of the individual classification regions of the plurality of classification regions corresponding to genomic regions of a reference genome that have the threshold amount of methylated cytosines in subjects in which cancer is detected and that have at least the threshold cytosine-guanine content. The operations also include analyzing the training sequencing reads to determine a second quantitative measure derived from the training sequencing reads that correspond to a plurality of control regions, individual control regions of the plurality of control regions corresponding to additional genomic regions of the reference genome that have at least the threshold cytosine-guanine content and that have at least the threshold amount of methylated cytosines in subjects in which cancer is detected and in additional subjects in which cancer is not detected. The operations also include determining a metric for the individual classification regions of the plurality of classification regions based on the first quantitative measure for the individual classification regions and the second quantitative measure for the plurality of control regions. The operations also include generating training data that includes the metric for the individual classification regions of the plurality of classification regions for the training sequence reads from samples of training subjects. The operations also include implementing, using the training data, one or more machine learning algorithms to generate a model to determine an indication of cancer status in subjects based on amounts of methylated cytosines in at least a portion of the plurality of classification regions, the model including weights for individual classification regions of the plurality of classification regions and at least a portion of the weights of the individual classification regions being different from one another.

In one or more aspects, a method includes obtaining a first sample from a subject and a second sample from the subject, obtaining, by a computing system having one or more hardware processors and memory, sequence data including sequencing reads derived from a plurality of samples of a subject, individual sequencing reads including a nucleotide sequence corresponding to a fragment of a nucleic acid included in a sample of the plurality of samples and individual training sequencing reads corresponding to molecules having a threshold amount of methylated cytosines included in regions of the nucleotide sequence having at least a threshold cytosine-guanine content. The method also includes analyzing, by the computing system, first sequencing reads included in the sequence data to determine first quantitative measures that correspond to individual first classification regions of a plurality of first classification regions, at least a portion of the individual first classification regions of the plurality of first classification regions corresponding to first genomic regions of a reference genome that have the threshold amount of methylated cytosines in subjects in which cancer is detected and that have at least the threshold cytosine-guanine content. The method also includes analyzing, by the computing system, second sequencing reads included in the sequence data to determine second quantitative measures that correspond to individual second classification regions of a plurality of second classification regions, at least a portion of the individual second classification regions of the plurality of second classification regions corresponding to second genomic regions of a reference genome that have the threshold amount of methylated cytosines in subjects in which cancer is detected and that have at least the threshold cytosine-guanine content. In addition, the method includes determining, by the computing system, one or more first classification regions that overlap with one or more second classification regions to produce third classification regions. Further, the method includes analyzing, by the computing system, at least one of the first quantitative measures or the second quantitative measures to determine an indication of cancer status in the subject. The indication of cancer status can include at least one of tumor fraction or minor allele frequency.

In one or more aspects, the method includes obtaining the first sample before at least one of a procedure or administration of a treatment for cancer and obtaining the second sample after at least one of the procedure or the administration of the treatment for cancer.

In one or more aspects, the method includes determining, by the computing system, the first quantitative measures by analyzing a number of first sequencing reads included in the sequence data that correspond to individual first classification regions in relation to a total number of the first sequencing reads that correspond to a group of the first classification regions. In one or more examples, the group of the first classification regions can include all of the first classification regions.

In one or more aspects, the method includes, determining, by the computing system, an individual first classification region by: determining, by the computing system, a number of the first sequencing reads that correspond to a genomic region of the reference genome, determining, by the computing system, a portion of the genomic region for which at least a threshold amount of the number of first sequencing reads overlap, and determining, by the computing system, that the portion of the genomic region corresponds to the individual first classification region. The genomic region can include a differentially methylated region. In one or more aspects, the threshold amount can include at least 70% of the number of first sequencing reads.

Described herein is a method for generating a tumor fraction estimate from a ceil-free deoxyribonucleic acid (cfDNA) sample of a subject, including receiving a dataset of methylation sequence reads from a cfDNA sample of a subject, determining at each of a plurality of one or more methylation levels and generating a methylation pattern over one or more CpG sites; comparing the plurality of variants to reference sequence reads to generate a subset of variants. In some embodiments the subset of variants is generated by comparison to reference sequence reads generated from non-cancer cfDNA samples. In some instances, the reference sequence reads are obtained from biopsy samples of a plurality of tissues of reference individuals. In various embodiments, for one or more variants, including in a subset a variant, a count of methylation sequence reads that include the variant; inputting the counts of methylation sequence reads for the variants of the subset to a model. In various embodiments, the model is trained based frequency rates of the plurality of variants. In some embodiments, the method includes generating a tumor fraction estimate of the cfDNA sample. In various embodiments, occurrence, recurrence or alterations rates of the plurality of variants are determined based on the reference sequence reads in the bank. In various embodiments, comparing the plurality of variants to reference sequence reads to generate a subset of variants comprises filtering out one or more variants whose rates of presence in the noncancer samples exceeds a threshold. In various embodiments, the particular occurrence, recurrence or alteration of a particular variant corresponds to a rate of observation of the particular variant among the reference sequence reads in the bank. In various embodiments, the tumor fraction prediction is a distribution of probability of a fraction of fragments in the cfDNA sample that are tumor derived. In various embodiments, the tumor fraction prediction is a fraction of fragments in the cfDNA sample that is tumor derived. In various embodiments, the model comprises at least one probabilistic model, the probabilistic model comprising a Poisson distribution for a particular variant, and the Poisson distribution is weighted by the recurrence rate of the particular variant. In various embodiments, the method includes a plurality of probabilistic distributions, each probabilistic distribution corresponding to a particular variant and parameterized based on a site-specific noise rate of the particular variant and per-site sequencing depth of the particular variant. In various embodiments, the h probabilistic distribution corresponding to a particular variant is further adjusted based on at least one of: a depth of the cfDNA sample, a binding panel efficiency of the cfDNA sample, and an estimated tumor fraction of the cfDNA sample. In various embodiments, the count for each variant of the filtered subset comprises a count of methylation sequence reads of the cfDNA sample that include the methylation pattern over the one or more CpG sites of the variant.

A system including instructions for processing the methods of any preceding embodiment.

4 computer readable medium including instructions for processing the methods of any preceding embodiment.

### DETAILED DESCRIPTION

Cancer is usually caused by the accumulation of mutations within genes of an individual's cells, at least some of which result in improperly regulated cell division. Such mutations can include single nucleotide variations (SNVs), gene fusions, insertions, transversions, translocations, and inversions. These mutations can also include copy number variations that correspond to an increase or a decrease in the number of copies of a gene within a tumor genome relative to an individual's noncancerous cells. An extent of mutations present in cell-free nucleic acids and an amount of mutated cell-free nucleic acids of a sample can be used as biomarkers to determine tumor progression, predict patient outcome, and refine treatment choices. In various examples, the extent of mutations present in cell-free nucleic acids can be indicated by tumor cells copy number and tumor fraction for a given sample.

Additionally, cancer can be indicated by non-sequence modifications, such as methylation. Examples of methylation changes in cancer include local gains of DNA methylation in the CpG islands at the TSS of genes involved in normal growth control, DNA repair, cell cycle regulation, and/or cell differentiation. This increased amount of methylation can be associated with an aberrant loss of transcriptional capacity of involved genes and occurs at least as frequently as point mutations and deletions as a cause of altered gene expression.

Thus, DNA methylation profiling can be used to detect aberrant methylation in DNA of a sample. The DNA can correspond to certain genomic regions (“differentially methylated regions” or “DMRs”) that are normally hypermethylated or hypomethylated in a given sample type (e.g., cfDNA from the bloodstream) but which may show an abnormal degree of methylation that correlates to a neoplasm or cancer, e.g., because of unusually increased contributions of tissues to the type of sample (e.g., due to increased shedding of DNA in or around the neoplasm or cancer) and/or from extents of methylation of the genome that are altered during development or that are perturbed by disease, for example, cancer or any cancer-associated disease.

Some methods of measuring DNA methylation, can make accurately determining an amount of methylation of DNA difficult. The accuracy with which DNA methylation is determined can impact the accuracy of estimates of tumor fraction for samples. Since tumor fraction can be used to determine whether a sample is derived from a subject in which a tumor is present or not, the accuracy of determination of tumor fraction estimates can impact diagnosis and/or treatment decisions for individuals.

The methods and systems described herein are directed to accurately generating information indicating the amounts of methylation of nucleic acids using data that indicates an amount of binding of nucleic acids to methyl binding domain (MBD). In various examples, the application is directed to systems and processes to determine an estimate for tumor fraction of a sample. In one or more examples, amounts of methylation of nucleic acids can be determined based on a strength of binding by the nucleic acids to methyl binding domain (MBD). The nucleic acids can be partitioned according to the strength of binding to MBD. Additionally, a number of cytosine-guanine (CG) regions for the nucleic acids can be determined. Amounts of methylation of classification regions of the nucleic acids can be determined based on the partition information associated with the nucleic acids and the number of cytosine-guanine regions of the nucleic acids. The classification regions can have differing amounts of methylation in tumor cells and non-tumor cells. The estimate for tumor fraction of the sample can be determined according to the amounts of methylation of the classification regions.

In at least some implementations, the methods, systems, techniques, and architectures can implement models that are configured to have at least one of parameters or weights that can be modified to more accurately fit to the methylation data provided to the models. The methods, systems, techniques, and architectures are also directed to implementing a number of optimization procedures during the training of the models to generate models that more accurately predict metrics indicating the presence or absence of tumors than other systems, methods, techniques, and architectures. Further, the methods, techniques, and processes used to generate the information used to produce the methylation data reduce the amount of noise present in the methylation data that leads to more accurate predictions of metrics that indicate the presence or absence of tumors than other methods, techniques, and processes.

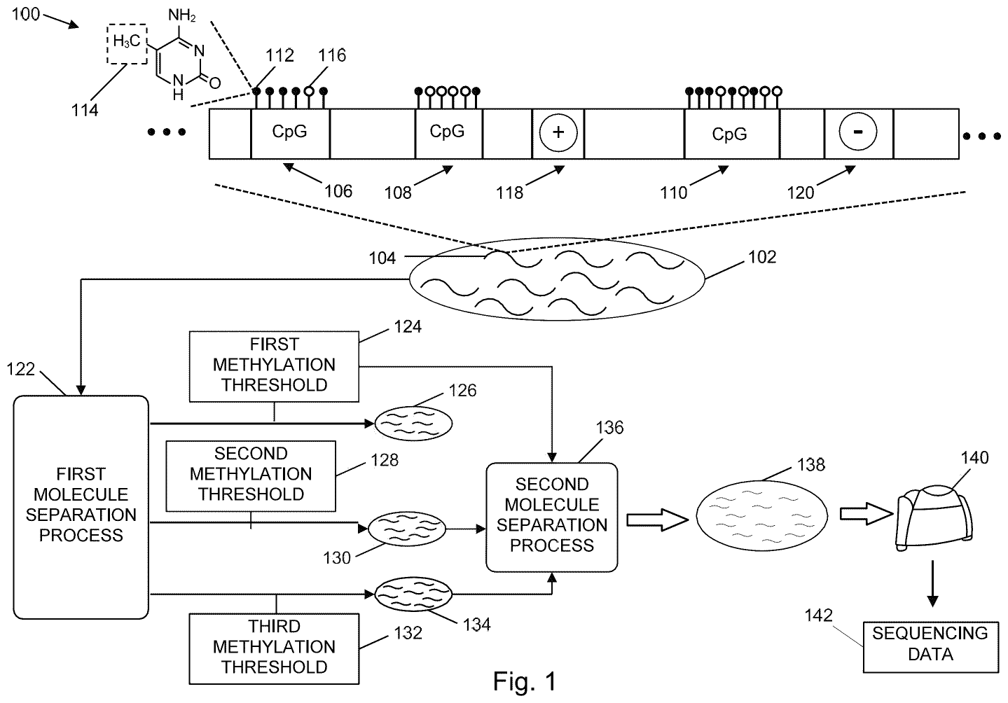

FIG. 1 is a diagrammatic representation of an example environment 100 that identifies nucleic acids that correspond to classification regions of a reference sequence, where the classification regions have at least a threshold number of CpGs, according to one or more implementations. In one or more examples, the disease under consideration is a type of cancer. Non-limiting examples of such cancers include biliary tract cancer, bladder cancer, transitional cell carcinoma, urothelial carcinoma, brain cancer, gliomas, astrocytomas, breast carcinoma, metaplastic carcinoma, cervical cancer, cervical squamous cell carcinoma, rectal cancer, colorectal carcinoma, colon cancer, hereditary nonpolyposis colorectal cancer, colorectal adenocarcinomas, gastrointestinal stromal tumors (GISTs), endometrial carcinoma, endometrial stromal sarcomas, esophageal cancer, esophageal squamous cell carcinoma, esophageal adenocarcinoma, ocular melanoma, uveal melanoma, gallbladder carcinomas, gallbladder adenocarcinoma, renal cell carcinoma, clear cell renal cell carcinoma, transitional cell carcinoma, urothelial carcinomas, Wilms tumor, leukemia, acute lymphocytic leukemia (ALL), acute myeloid leukemia (AML), chronic lymphocytic (CLL), chronic myeloid (CML), chronic myelomonocytic (CMML), liver cancer, liver carcinoma, hepatoma, hepatocellular carcinoma, cholangiocarcinoma, hepatoblastoma, Lung cancer, non-small cell lung cancer (NSCLC), mesothelioma, B-cell lymphomas, non-Hodgkin lymphoma, diffuse large B-cell lymphoma, Mantle cell lymphoma, T cell lymphomas, non-Hodgkin lymphoma, precursor T-lymphoblastic lymphoma/leukemia, peripheral T cell lymphomas, multiple myeloma, nasopharyngeal carcinoma (NPC), neuroblastoma, oropharyngeal cancer, oral cavity squamous cell carcinomas, osteosarcoma, ovarian carcinoma, pancreatic cancer, pancreatic ductal adenocarcinoma, pseudopapillary neoplasms, acinar cell carcinomas. Prostate cancer, prostate adenocarcinoma, skin cancer, melanoma, malignant melanoma, cutaneous melanoma, small intestine carcinomas, stomach cancer, gastric carcinoma, gastrointestinal stromal tumor (GIST), uterine cancer, or uterine sarcoma.

The environment 100 can include a sample 102. The sample 102 can be derived from a biological fluid obtained from a subject. For example, the sample 102 can be derived from blood obtained from a subject. In one or more additional examples, the sample 102 can be derived from tissue of a subject. In various examples, the sample 102 can be derived from multiple sources. To illustrate, the sample 102 can be derived from one or more fluids of a subject and/or from tissue of a subject. In one or more illustrative examples, the subject can be a mammal. In one or more additional illustrative examples, the subject can be a human. In one or more further illustrative examples, the subject can be a non-human mammal.

The sample 102 can include a number of nucleic acids 104. Individual nucleic acids 104 can include a number of regions that have at least a threshold number of cytosine molecules and guanine molecules. In one or more examples, individual nucleic acids 104 can include regions having at least a threshold number of cytosine-guanine dinucleotides. In various examples, at least a portion of the cytosine-guanine pairs included in the regions can be sequentially located in sequences of the nucleic acids 104. In one or more illustrative examples, a region of a nucleic acid having at least a threshold amount of cytosine-guanine pairs can be referred to herein as a “CG region” or a “CpG region.” In one or more examples, a CG region can include at least 200 CpG dinucleotides. In one or more illustrative examples, a CG region can include from 200 CpG dinucleotides to 5000 CpG dinucleotides, from 300 CpG dinucleotides to 3000 CpG dinucleotides, from 200 CpG dinucleotides to 2500 CpG dinucleotides, or from 500 CpG dinucleotides to 1500 CpG dinucleotides. Additionally, a CG region can have a GC percentage of at least 50% and an observed-to-expected CpG ratio of at least 60%. The observed-to-expected CpG ratio can be calculated where the observed CpG is the number of CpGs identified in a given genomic region and the expected CpGs is the number of cytosines multiplied by the number of guanines divided by the number of bases in the genomic region. The expected CpGs can also be calculated by:

((number of cytosines+number of guanines)/2)²/length of genomic region.

For example, a CG region can be determined using the techniques described by Gardiner-Garden M, Frommer M (1987). “CpG islands in vertebrate genomes”. Journal of Molecular Biology. 196 (2): 261-282. and/or Saxonov S, Berg P, Brutlag D L (2006). “A genome-wide analysis of CpG dinucleotides in the human genome distinguishes two distinct classes of promoters”. Proc Natl Acad Sci USA. 103 (5): 1412-1417.

In the illustrative example of FIG. 1, a portion of a sequence of an example nucleic acid 104 can include a first CG region 106, a second CG region 108, and a third CG region 110. Although the illustrative example of FIG. 1 illustrates a portion of a sequence of a nucleic acid 104 having three CG regions, nucleic acids 104 included in the sample 102 can have a different number of CG regions. For example, individual nucleic acids 104 included in the sample 102 can include at least 1 CG region, at least 5 CG regions, at least 10 CG regions, at least 25 CG regions, at least 50 CG regions, at least 100 CG regions, at least 250 CG regions, at least 500 CG regions, or at least 1000 CG regions.

Individual CG regions can correspond to a number of molecules with one or more methylated cytosines. In the illustrative example of FIG. 1, the CG region 106 can include a molecule with a methylated cytosine 112. In the illustrative example of FIG. 1, the molecule with a methylated cytosine 112 is 5-methylcytosine. Individual CG regions can also correspond to a number of molecules with an unmethylated cytosine. For example, the CG region 106 can include a molecule with an unmethylated cytosine 116. In various examples, at least a portion of the CG regions of a nucleic acid 104 can correspond to classification regions of a reference genome. Classification regions can correspond to genomic regions of a reference genome that correspond to non-sequence differences that are consistent with one or more biological conditions, such as one or more types of cancer. In at least some examples, the non-sequence differences can include one or more mutations that are consistent with one or more biological conditions. In one or more examples, a classification region can correspond to a genomic region of the reference sequence for which molecules derived from subjects having at least one form of cancer. In at least some examples, nucleic acid molecules having at least a threshold amount of methylated cytosines in at least one CG region (e.g., hypermethylated molecules) can be derived from subjects in which cancer is present and correspond to a classification. In one or more additional examples, nucleic acid molecules having less than a threshold amount of methylated cytosines (e.g., hypomethylated molecules) in at least one CG region can be derived from subjects in which cancer is present and correspond to a classification region.

In addition to the classification regions, the CG regions can include one or more positive control regions, such as positive control region 118. The positive control region 108 can be mapped to nucleic acid molecules having at least a threshold number of methylated cytosine molecules in at least one CG region and that are derived from subjects that are free of cancer and are derived from subjects in which cancer is present. In various examples, the positive control region 106 can be hypermethylated in cells derived from subjects that are free of cancer and also in cells derived from subjects in which cancer is present. The CG regions can also include one or more negative control regions, such as negative control region 120. The negative control region 120 can be mapped to nucleic acid molecules having less than a threshold number of methylated cytosine molecules in at least one CG region and that are derived from subjects that are free of cancer and also subjects in which cancer is present. In one or more illustrative examples, the negative control region 120 can be hypomethylated in subjects that are free of cancer and also in subjects in which cancer is present. In various examples, the positive control regions and the negative control regions can be used to perform normalization calculations. The normalization calculations can be performed to generate input data for one or more models that are implemented to determine tumor metrics for a given sample 102.

A first molecule separation process 122 can be performed. The first molecule separation process 122 can separate nucleic acids 104 included in the sample 102 based on an amount of methylated cytosines of the individual nucleic acids 104. In one or more examples, the first molecule separation process can separate nucleic acids 104 included in the sample 102 based on amounts of methylated cytosines included in CG regions of individual nucleic acids 104. In various examples, the first molecule separation process 122 can separate the nucleic acids 104 into a plurality of groups with individual groups corresponding to respective amounts of methylated cytosines of the nucleic acids 104.

In the illustrative example of FIG. 1, the first molecule separation process 122 can be performed in relation to a first methylation threshold 124. Performing the first molecule separation process 122 with regard to the first methylation threshold 124 can produce a first partition of nucleic acids 126. In one or more examples, the first methylation threshold 124 can indicate a first threshold number of molecules with a methylated cytosine located in CG regions of the nucleic acids 104. The first molecule separation process 122 can identify a number of nucleic acids 104 having fewer molecules with a methylated cytosine in CG regions than the first methylation threshold 124. In various examples, the first methylation threshold 124 can correspond to a first methylation rate.

The first molecule separation process 122 can also be performed with respect to a second methylation threshold 128. The second methylation threshold 128 can indicate an amount of methylated cytosines in one or more genomic regions of the nucleic acids 104 that is greater than the amount of methylated cytosines in the one or more regions corresponding to the first methylation threshold 124. The second methylation threshold 124 can indicate a number of molecules with a methylated cytosine per a number of nucleic acids. In one or more additional examples, the second methylation threshold 124 can correspond to a rate of methylation of nucleic acids that is greater than the rate of methylation that corresponds to the first methylation threshold 124. Performing the first molecule separation process 122 with respect to the second methylation threshold 128 can produce a second partition of nucleic acids 130. In one or more examples, the first molecule separation process 122 can identify nucleic acids 104 having a greater amount of methylated cytosines than the first methylation threshold 124 and having a lower amount of methylated cytosines than the second methylation threshold 128 to produce the second partition of nucleic acids 130.

Additionally, the first molecule separation process 122 can also be performed with respect to a third methylation threshold 132. The third methylation threshold 132 can indicate an amount of methylated cytosines in one or more genomic regions of the nucleic acids 104 that is greater than the amount of methylated cytosines in the one or more regions corresponding to the first methylation threshold 124 and greater than the amount of methylated cytosines in the one or more regions corresponding to the second methylation threshold 128. The third methylation threshold 132 can indicate a number of molecules with a methylated cytosine per a number of nucleic acids. In one or more additional examples, the third methylation threshold 132 can correspond to a rate of methylated cytosines that is greater than the rate of methylation that corresponds to the first methylation threshold 124 and greater than the rate of methylation that corresponds to the second methylation threshold 128. Performing the first molecule separation process 122 with respect to the third methylation threshold 132 can produce a third partition of nucleic acids 134. In one or more examples, the first molecule separation process 122 can identify nucleic acids 104 having a greater amount of methylated cytosines than nucleic acids 104 included in the second partition of nucleic acids 128. In this way, the amount of methylated cytosines of nucleic acids included in the first partition 122, the second partition 126, and the third partition 130 increases from the first partition 122 to the second partition 126 and increases from the second partition 126 to the third partition 130. In one or more illustrative examples, the first partition of nucleic acids 126 can be referred to as a hypomethylation partition, the second partition of nucleic acids 130 can be referred to as an intermediate partition, and the third partition of nucleic acids 134 can be referred to as a hypermethylation partition.

In one or more examples, the amount of methylated cytosines of nucleic acids can correspond to a strength of binding to methyl binding domain (MBD). In these scenarios, the first partition 126, the second partition 130, and the third partition 134 can be produced based on different strengths of binding to MBD for nucleotides having different amounts of methylated cytosines. In one or more examples, the first molecule separation process 122 can include a series of washes where the nucleic acids 104 are contacted with solutions having different concentrations of sodium chloride (NaCl).

Partitioning of the nucleic acids can be performed by contacting the nucleic acids with a modified nucleotide specific binding reagent, such as a MBD of a MBP. A modified nucleotide specific binding reagent can bind to 5-methylcytosine (5mC). The modified nucleotide specific binding reagent, such as a MBD, can be coupled to paramagnetic beads, such as Dynabeads® M-280 Streptavidin via a biotin linker. Partitioning into fractions with different extents of methylation can be performed by increasing the NaCl concentration in a series of washes. The sequences eluted from the modified nucleotide specific binding reagent are partitioned into two or more fractions (e.g., hypo, hyper) depending on which wash (e.g., NaCl concentration) eluted the sequences. Resulting partitions can include one or more of the following nucleic acid forms: double-stranded DNA (dsDNA), shorter DNA fragments and longer DNA fragments.

The binding of the nucleic acids with the modified nucleotide specific binding reagent can be a function of number of methylated (or modified) sites per molecule, with molecules having more methylation eluting under increased salt concentrations. To elute the DNA into distinct populations based on the extent of methylation, one can use a series of elution buffers of increasing NaCl concentration. Salt concentrations can, in one or more implementations, range from about 100 nM to about 2500 mM NaCl. In various implementations, the process results in three (3) partitions. Molecules are contacted with a solution at a first salt concentration and comprising a molecule comprising a methyl binding domain, which molecule can be attached to a capture moiety, such as streptavidin. At the first salt concentration a population of molecules will bind to the MBD and a population will remain unbound. The unbound population can be separated as a “hypomethylated” population (hypo partition). For example, the first partition 126 can be representative of the hypomethylated form of DNA is that which remains unbound at a low salt concentration. In one or more illustrative examples, the concentration of NaCl of the solution used to produce the first partition 126 can be about 100 nM, about 120 nM, about 140 nM, about 160 nM, about 180 nM, about 200 nM. or about 250 nM. The second partition 130 can be referred to as a “residual partition” or an “intermediate partition” and can be representative of intermediate methylated DNA is eluted using an intermediate salt concentration, e.g., between 100 mM and 2000 mM concentration. In one or more additional illustrative examples, the concentration of NaCl of the solution used to produce the second partition 130 can be from about 100 mM to about 500 mM, from about 100 mM to about 1000 mM, from about 100 mM to about 1500 mM, from about 250 mM to about 1000 mM, from about 250 mM to about 1500 mM, from about 500 mM to about 1500 mM, from about 250 mM to about 2000 mM, from about 500 mM to about 2000 mM, or from about 1000 mM to about 2000 mM. This is also separated from the sample. The third partition 134 can be representative of hypermethylated form of DNA (hyper partition) and is eluted using a high salt concentration, e.g., at least about 2000 mM. In one or more further illustrative examples, the concentration of NaCl of the solution used to produce the third partition 134 can be from about 2000 mM to about 5000 mM, from about 2000 mM to about 4000 mM, from about 2000 mM to about 3500 mM, from about 2000 mM to about 3000 mM, or from about 2500 mM to about 4000 mM.

In various examples, the first partition 126 can correspond to a first range of binding strengths of nucleic acids to MBD and to a first range of methylated CG regions and the second partition 130 can correspond to a second range of binding strengths of nucleic acids to MBD and to a second range of methylated CG regions. The first range of binding strengths can be less than the second range of binding strengths. In one or more scenarios, a first solution having a first NaCl concentration can separate a first group of nucleic acids having the first range of binding strengths from MBD and a second solution having a second NaCl concentration can separate a second group of nucleic acids having the second range of binding strengths from MBD with the second NaCl concentration being greater than the first NaCl concentration. Additionally, the third partition 134 can correspond to a third range of binding strengths and a third range of methylated CG regions. The third range of binding strengths can be greater than the first range of binding strengths and the second range of binding strengths. In one or more instances, a third solution having a third NaCl concentration can separate a third group of nucleic acids having the third range of binding strengths from NaCl. The third NaCl concentration can be greater than the first NaCl concentration and the second NaCl concentration.

In one or more illustrative examples, a plurality of nucleic acids derived from at least one of blood or tissue of a subject can be combined with a solution including an amount of MBD to produce a nucleic acid-MBD solution. A first wash of the nucleic acid-MBD solution can be performed with a first solution including a first NaCl concentration to produce a first nucleic acid fraction and a first residual solution. The first nucleic acid fraction can include a first portion of the plurality of nucleic acids and the first residual solution can include a second portion of the plurality of nucleic acids. In one or more examples, the first portion of the plurality of nucleic acids can have a first range of binding strengths to MBD that are less than a second range of binding strengths to MBD of the second portion of the plurality of nucleic acids.

Additionally, a second wash of the first residual solution can be performed with a second solution including a second concentration of NaCl that is greater than the first concentration of NaCl to produce a second nucleic acid fraction and a second residual solution. The second nucleic acid fraction can include a first subset of the second portion of the plurality of nucleic acids and the second residual solution can include a second subset of the second portion of the plurality of nucleic acids. The first subset of the second portion of the plurality of nucleic acids can have a third range of binding strengths to MBD that are less than a fourth range of binding strengths to MBD of the second subset of the second portion of the plurality of nucleic acids. Further, a third wash of the second residual solution can be performed with a third solution including a third concentration of NaCl that is greater than the second concentration of NaCl to produce a third nucleic acid fraction that includes the second subset of the second portion of the plurality of nucleic acids.

Subsequent to the first wash, the second wash, and the third wash a determination can be made that the first portion of the plurality of nucleic acids are associated with the first partition 126. The first portion of the plurality of nucleic acids can be attached with a first set of molecular barcodes indicating the first partition 126. In this way, a sequencing read that corresponds to the first partition 126 can be identified based on determining that the sequencing read includes the first molecular barcode. In addition, a determination can be made that the first subset of the second portion of the plurality of nucleic acids is associated with an additional partition of the plurality of partitions. In these situations, a second set of molecular barcodes different from the first set of molecular barcodes can be attached to the second portion of the plurality of nucleic acids with the second molecular barcode indicating the additional partition. As a result, a sequencing read that corresponds to the additional partition can be identified based on determining that the sequencing read includes a second bar code from among the second set of molecular barcodes. Further, a determination can be made that the second subset of the second portion of the plurality of nucleic acids is associated with the second partition 130. A third set of molecular barcodes different from the first set of molecular barcodes and the second set of molecular barcodes can then be attached to the second subset of the second portion of the plurality of nucleic acids where the third set of molecular barcodes indicate the second partition 130. In these instances, a sequencing read that corresponds to the second partition 130 can be identified based on determining that the sequencing read includes a third molecular barcode from among the third set of molecular barcodes.

In at least some examples, the first molecule separation process 122 can result in nucleic acids being present in at least one of the first partition 126, the second partition 130, or the third partition 134 having an amount of methylation that is different from the amount of methylation of the other nucleic acids in the respective partition. For example, the first partition 126 can include a number of nucleic acids having amounts of methylation that correspond to the amounts of methylation of nucleic acids included in at least one of the second partition 130 or the third partition 134. Additionally, at least one of the second partition 130 or the third partition 134 can include nucleic acids having amounts of methylation that correspond to the amounts of methylation of nucleic acids included in the first partition 126. The presence of nucleic acids in at least one of the first partition 126, the second partition 130, or the third partition 134 that do not correspond to the amounts of methylation of at least a majority of the other nucleic acids included in the respective partition can cause data noise when performing computational operations with respect to sequence reads produced from nucleic acids included in the first partition 126, the second partition 130, and the third partition 134. The data noise can result in inaccuracies with respect to calculations made based on sequence reads derived from nucleic acids included in the first partition 126, the second partition 130, and the third partition 134.

To reduce or eliminate data noise associated with nucleic acids being present in at least one of the first partition 126, the second partition 130, or the third partition 134 that have amounts of methylation that are not consistent with the amounts of methylation of at least a majority of other molecules included in the respective partitions, a second molecule separation process 136 can be performed after the first molecule separation process 122. The second molecule separation process 136 can be performed with respect to nucleic acids included in the first partition 126, nucleic acids included in the second partition 130, and nucleic acids included in the third partition 134. In one or more examples, the second molecule separation process 136 can include performing digestion of the nucleic acids included in the first partition 126 using methylation dependent restriction enzyme (MDRE) and nucleic acids included in the second partition 130 and the third partition 134 can be digested using methyationl sensitive restriction enzyme (MSRE). Digestion of the nucleic acids included in the first partition 126 with MDRE can result in separation of nucleic acids included in the first partition having amounts of methylation corresponding to the second partition 130 and the third partition 134 from nucleic acids having amounts of methylation corresponding to the first partition. Additionally, digestion of nucleic acids included in the second partition 130, and the third partition 134 with MSRE can result in separation of the nucleic acids having amounts of methylation corresponding to the first partition 126 from the nucleic acids of the second partition 130 and the nucleic acids of the third partition 134. By removing nucleic acids from the first partition 126 having amounts of methylation that correspond to the second partition 130 and the third partition 134 and by removing nucleic acids from the second partition 130 and the third partition 134 that have amounts of methylation that correspond to the first partition 126, an additional group of nucleic acids 138 can be produced. The additional group of nucleic acids 138 can include nucleic acids corresponding to methylation amounts of the second partition 130 and the third partition 134 with a minimal amount or no nucleic acids having amounts of methylation corresponding to the first partition 126. For example, at least 95% of the nucleic acids included in the additional group 138 can have amounts of methylation that correspond to the second partition 130 and the third partition 134, at least 97% of the nucleic acids included in the additional group 138 can have amounts of methylation that correspond to the second partition 130 and the third partition 134, at least 99% of the nucleic acids included in the additional group 138 can have amounts of methylation that correspond to the second partition 130 and the third partition 134, at least 99.5% of the nucleic acids included in the additional group 138 can have amounts of methylation that correspond to the second partition 130 and the third partition 134, or at least 99.9% of the nucleic acids included in the additional group 138 can have amounts of methylation that correspond to the second partition 130 and the third partition 134.

The architecture 100 can include a sequencing machine 140. In one or more examples, the sequencing machine 140 can be any of a number of sequencing machines that can perform one or more sequencing operations that amplify nucleic acids present in a sample 104. In various examples, the sequencing machine 140 can perform next-generation sequencing operations. In one or more examples, the sample 104 can include an amount of at least one bodily fluid extracted from a subject. In one or more additional examples, the sample 104 can include a tissue sample that is obtained from a subject.

In one or more examples, prior to sequencing, the extracted polynucleotides can be partitioned into two or more partitions based on the binding strength of the of binding strengths of polynucleotides to MBD. A blunt-end ligation can be performed on the partitioned polynucleotides and adapters, as well as tags (e.g., molecular barcodes) can be added to the partitioned polynucleotides. The tagged polynucleotides in the one or more partitions (e.g. hyper and/or intermediate partitions) can be treated with one or more methylation sensitive restriction enzymes (MSREs). Post the MSRE treatment, the molecules can also be enriched by causing hybridization between the extracted polynucleotides and probes that correspond to target regions of a reference sequence. The enrichment process can identify thousands, hundreds of thousands, up to millions of polynucleotides that correspond to on-target regions associated with the probes. Thousands, up to millions of unenriched polynucleotides that correspond to off-target regions of the reference sequence can also be present after the enrichment process.

Subsequent and/or prior to the enrichment process, the molecules can be amplified according to one or more amplification processes. The one or more amplification processes can produce thousands, up to millions of copies of individual nucleic acid molecules. In one or more examples, a portion of the unenriched polynucleotides can be amplified, in some instances, but not to the extent that the enriched polynucleotides are amplified. The one or more amplification processes can generate an amplification product that undergoes one or more sequencing operations. After performing one or more sequencing operations with respect to the sample 104, the sequencing machine 140 can produce a sequencing data 142.

The sequencing data 142 can include alphanumeric representations of the nucleic acids included in an amplification product. For example, the sequencing data 142 can include, for individual nucleic acids of the amplification product, data that corresponds to a string of letters that represent the respective chains of nucleotides that correspond to the individual nucleic acids.

The sequencing data 142 can be stored in one or more data files. For example, the sequencing data 142 can be stored in a FASTQ file that includes a text-based sequencing data file format storing raw sequence data and quality scores. In one or more additional examples, the sequencing data 142 can be stored in a data file according to a binary base call (BCL) sequence file format. In one or more further examples, the sequencing data 142 can be stored in a BAM file. In one or more examples, the sequencing data 142 can comprise at least about one gigabyte (GB), at least about 2 GB, at least about 3 GB, at least about 4 GB, at least about 5 GB, at least about 8 GB, or at least about 10 GB. An individual sequence representation included in the sequencing data 106 can be referred to herein as a “read” or a “sequencing read.” In various examples, individual first nucleic acids included in the pool 138 can correspond to multiple sequence representations included in the sequencing data 142 as a result of the amplification of the individual first nucleic acids. In one or more additional examples, individual second nucleic acids included in the pool 138 can correspond to a single sequence representation included in the sequencing data 142 as a result of the absence of amplification of the individual second nucleic acids.

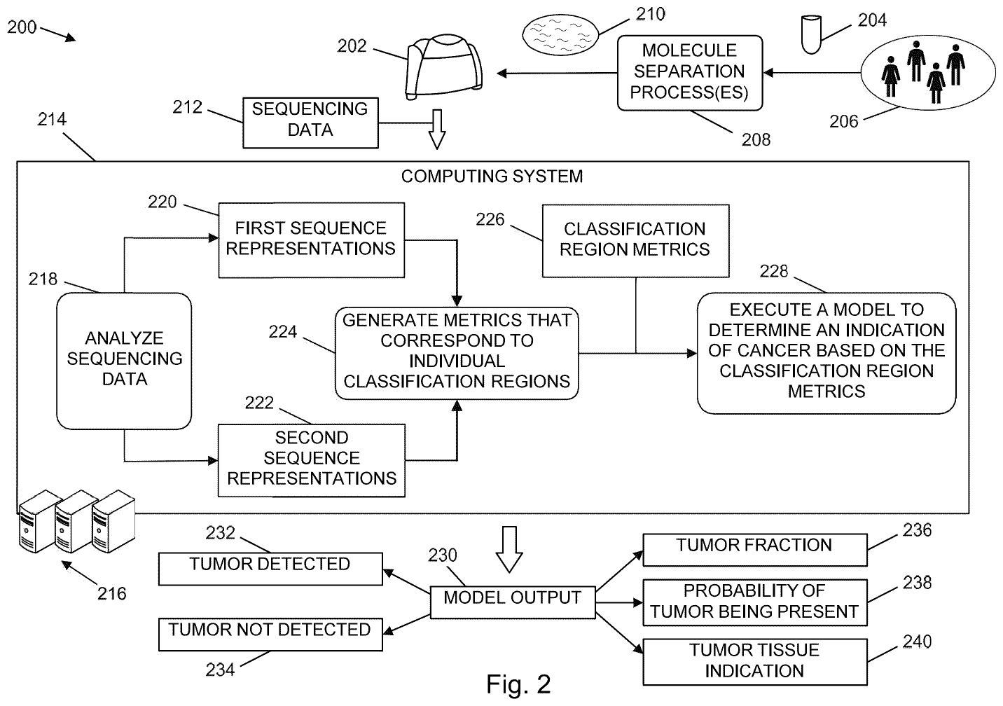

FIG. 2 is an example architecture 200 to analyze sequencing data to determine one or more metrics indicating the presence of a tumor in subjects, in accordance with one or more implementations. The architecture 200 can include one or more sequencing machines 202 that perform one or more sequencing operations with respect to a number of samples 204. The one or more samples 204 can be obtained from subjects 206. In one or more illustrative examples, a first portion of the subjects 206 can be free of cancer. That is, a tumor is not detected in the first portion of the subjects 206. Additionally, a tumor can be present in a second portion of the subjects 206.

One or more molecule separation processes 208 can be performed with respect to the samples 204. The one or more separation processes 208 can correspond to separating nucleic acid molecules into a number of partitions based on the characteristics of the nucleic acid molecules. Examples of characteristics that can be used for partitioning nucleic acid molecules include multiple different nucleotide modifications, methylation level, nucleosome binding, sequence mismatch, immunoprecipitation, and/or proteins that bind to DNA. In one or more illustrative examples, a heterogeneous population of nucleic acid molecules can be partitioned into nucleic acid molecules with one or more epigenetic modifications and without the one or more epigenetic modifications. Examples of epigenetic modifications include, but are not limited to, presence or absence of methylation; level of methylation, hydroxymethylation, and type of methylation (5′ cytosine or 6 methyladenine).

Prior to the one or more molecule separation processes 208, nucleic acid molecules can be extracted from a sample 204. In one or more implementations, the nucleic acid molecules comprise cell-free nucleic acids (e.g., cell-free DNA). In various implementations, the sample 204 can be a sample selected from one or more of blood, plasma, serum, urine, fecal, saliva samples, combinations thereof, and/or the like. In one or more additional examples, the sample 204 can comprise a sample selected from one or more of whole blood, a blood fraction, a tissue biopsy, pleural fluid, pericardial fluid, cerebrospinal fluid, and peritoneal fluid. In one or more illustrative examples, the cell-free nucleic acid molecules can be extracted from the sample 204 where the sample 204 is obtained from a subject 206 known to have cancer (e.g., a cancer patient), or a subject 206 suspected of having cancer.

The extraction of nucleic acid molecules from the sample 204 can include implementing one or more cell lysis techniques to cleave the membranes of cells included in the sample 204 and applying one or more proteases to break down proteins included in the sample 204. The extraction of nucleic acid molecules from the sample 204 can also include a number of washing and/or elution techniques to separate the nucleic acid molecules from other components included in the sample 204. In various examples, thousands, up to millions, up to billions of nucleic acid molecules can be extracted from the sample 204 prior to being subjected to the one or more separation processes 208.

The nucleic acid molecules extracted from samples 204 can include molecules having varying levels of methylation. Methylation can occur from any one or more post-replication or transcriptional modifications. Post-replication modifications include modifications of the nucleotide cytosine, including, but not limited to, 5-methylcytosine, 5-hydroxymethylcytosine, 5-formylcytosine and 5-carboxylcytosine. The one or more molecule separation processes 208 can separate nucleic acid molecules extracted from samples 204 into a number of partitions with individual partitions corresponding to different levels of methylation. For example, the molecule separation processes 208 can produce a first partition of nucleic acid molecules having first levels of methylation, a second partition of nucleic acid molecules having second levels of methylation, and a third partition of nucleic acid molecules having third levels of methylation. In various examples, the second levels of methylation can be greater than the first levels of methylation and the third levels of methylation can be greater than the first levels of methylation and the second levels of methylation. In one or more illustrative examples, the one or more molecule separation processes 208 can include the first molecule separation process 122 and the second molecule separation process 136 of FIG. 1.

The one or more molecule separation processes 208 can produce a pool 210 that includes a portion of the nucleic acid molecules extracted from one or more samples 204 and subjected to the one or more molecule separation processes 208. For example, the pool 210 can include a number of nucleic acid molecules having the second levels of methylation and a number of nucleic acid molecules having the third levels of methylation. Thus, the nucleic acid molecules included in the pool 210 can have at least a threshold amount of methylation. In one or more illustrative examples, the nucleic acid molecules included in the pool 210 can have at least a threshold amount of methylation in CG regions of the nucleic acid molecules.

The one or more sequencing machines 202 can perform one or more sequencing operations to produce sequencing data 212 that corresponds to the pool 210. The architecture 200 can include a computing system 214 that obtains the sequencing data 212 from the one or more sequencing machines 202 and analyzes the sequencing data 212. For example, the computing system 214 can analyze the sequencing data 212 to determine one or more metrics indicating that a tumor may be present in a subject 206 that provided at least one sample 204. The computing system 214 can include one or more computing devices 216. The one or more computing devices 216 can include at least one of one or more desktop computing devices, one or more mobile computing devices, or one or more server computing device. In various examples, at least a portion of the one or more computing devices 216 can be included in a remote computing environment, such as a cloud computing environment. In one or more examples, the computing system 214 and the sequencing machine 202 can be owned, operated, maintained, and/or controlled by a single organization. In one or more additional examples, the computing system 214 and the sequencing machine 202 can be owned, operated, maintained, and/or controlled by multiple organizations.

At operation 218, the computing system 214 can analyze the sequencing data 212. Analyzing the sequencing data 212 can include determining one or more first sequence representations 220 included in the sequencing data 212 that correspond to one or more classification regions of a reference sequence. The one or more classification regions can correspond to genomic regions of a reference sequence that are mapped to nucleic acid molecules having an amount of methylation in cfDNA obtained from subjects in which cancer is present relative to an amount of methylation of the molecules that map to the same genomic regions of the reference sequence in cfDNA obtained from subjects in which a tumor is not present. In at least some examples, the amount of methylation present in nucleic acid molecules that map to a classification region and are derived from subjects in which cancer is present is less than the amount of methylation present in nucleic acid molecules that map to the classification region and are derived from subjects in which cancer is not present. In one or more additional examples, the amount of methylation present in nucleic acid molecules that map to a classification region and are derived from subjects in which cancer is present is greater than the amount of methylation present in nucleic acid molecules that map to the classification region and are derived from subjects in which cancer is not present. The one or more classification regions can also include at least a threshold amount of cytosine-guanine content. In various examples, the one or more classification regions can include a series of cytosine-guanine (CG) pairs in the 5′→*3′ direction (CpG sites), such as at least 3 CpG sites, at least 5 CpG sites, at least 8 CpG sites, at least 10 CpG sites, at least 12 CpG sites, at least 15 CpG sites, at least 18 CpG sites, or at least 20 CpG sites.

In addition, the computing system 214 can analyze the sequencing data 212 to determine one or more second sequence representations 222 that correspond to one or more control regions of a reference sequence. The one or more control regions can include one or more positive control regions and/or one or more negative control regions. In various examples, a positive control region can comprise a genomic region of a reference sequence having at least a threshold amount of molecules with a methylated cytosine and including at least a threshold number of CpG sites. A positive control region can correspond to nucleic acid molecules having at least a threshold amount of methylation in one or more CG regions and that are obtained from subjects in which cancer is present and in samples obtained from subjects in which a tumor is not present. In one or more illustrative examples, positive control regions can be mapped to nucleic acid molecules that are hypermethylated in one or more CG regions and are derived from samples obtained from both subjects in which cancer is present and subjects in which cancer is not present. In one or more examples, a negative control region can comprise a genomic region of a reference sequence having less than a threshold amount of molecules with a methylated cytosine and at least a threshold number of CpG sites. A negative control region can correspond to nucleic acid molecules having less than an additional threshold amount of methylation in one or more CG regions and that are obtained from subject in which cancer is present and in samples obtained from subjects in which a tumor is not present. In one or more additional illustrative examples, negative control regions can be mapped to nucleic acid molecules that are hypomethylated in one or more CG regions and are derived from samples obtained from both subjects in which cancer is present and subjects in which cancer is not present.

In one or more illustrative examples, the first sequence representations 220 can be determined by aligning sequence representations included in the sequencing data 212 with one or more classification regions of a reference sequence. In addition, the second sequence representations 222 can be determined by aligning sequence representations included in the sequencing data 212 with one or more control regions of a reference sequence. The alignment process can identify the first sequence representations 220 by determining a number of sequence representations included in the sequencing data 212 that correspond to one or more classification regions of the reference sequence. Further, the alignment process can identify the second sequence representations 222 by determining a number of sequence representations that correspond to one or more control regions of the reference sequence.

In one or more illustrative examples, the alignment process can determine an amount of homology between individual sequence representations included in the sequence data 212 and portions of the reference sequence. The amount of homology between a given sequence representation and the reference sequence can indicate a number of positions of the reference sequence that have the same nucleotide as corresponding positions of the given sequence representation. The computing system 214 can determine that a sequence representation is aligned with a portion of a reference sequence based on determining that the sequence representation and the portion of the reference sequence have at least a threshold amount of homology. In scenarios where a sequence representation has at least the threshold amount of homology with respect to multiple portions of the reference sequence, the portion of the reference sequence having the greatest amount of homology with the sequence representation can be determined to be aligned with the sequence representation.

The amount of homology between a given sequence representation and a portion of a reference sequence can be determined using BLAST programs (basic local alignment search tools) and PowerBLAST programs (Altschul et al., J. Mol. Biol., 1990, 215, 403-410; Zhang and Madden, Genome Res., 1997, 7, 649-656) or by using the Gap program (Wisconsin Sequence Analysis Package, Genetics Computer Group, University Research Park, Madison Wis.), using default settings, which uses the algorithm of Needleman and Wunsch (J. Mol. Biol. 48; 443-453 (1970)). The amount of homology between a sequence representation and a portion of the reference sequence can also be determined using a Burrows-Wheeler aligner (Li, H., & Durbin, R. (2009). Fast and accurate short read alignment with Burrows-Wheeler transform. Bioinformatics, 25(14), 1754-1760).

In one or more examples, after the sequence representations included in the sequencing data 212 have been aligned with a reference sequence, the aligned sequence representations can be analyzed to identify one or more groups of sequence representations. For example, individual aligned sequence representations can correspond to individual sequencing reads that are included in the sequencing data 212. In these scenarios, the aligned sequence representations can include multiple reads that correspond to a single nucleic acid molecule included in the sample pool 210. In one or more additional examples, the aligned sequence representations can correspond to individual nucleic acid molecules included in the pool 210. In these situations, the computing system can determine a group of reads included in the sequence data 212 that correspond to an individual nucleic acid molecules included in the pool 210 based on molecular barcodes that are common to each group of sequencing reads. That is, individual nucleic acid molecules included in the pool 210 can be encoded with molecular barcodes that uniquely identify the individual nucleic acid molecules and, in at least some cases, the individual nucleic acid molecules can be represented by multiple sequencing reads included in the sequencing data 212. Accordingly, when multiple sequence representations are present in the sequencing data 212 that correspond to a single nucleic acid molecule included in the pool 210, the computing system 214 can group the multiple sequence representations together. In various examples, the groups of sequence representations that correspond to a single nucleic acid molecule included in the pool 210 can be referred to herein as “families.” Additionally, start and stop positions with respect to the reference sequence of the aligned sequence representations having a common molecular barcode can be used to group the sequence representations that correspond to individual nucleic acids included in the pool 210. In one or more illustrative examples, an individual sequence representation that represents a family of sequence representations that corresponds to a single nucleic acid molecule included in the pool 210 can be referred to herein as a “consensus sequence representation.”

At operation 224, the computing system 214 can analyze the first sequence representations and the second sequence representations 222 to generate metrics that correspond to individual classification regions. In the illustrative example of FIG. 2, the computing system 214 can analyze the first sequence representations 220 and the second sequence representations 222 to generate classification region metrics 226. The classification region metrics 226 can include quantitative measures determined based on a number of first sequence representations 220 having at least a threshold amount of methylated cytosines. In one or more illustrative examples, the classification region metrics 226 can include quantitative measures determined based on a number of sequencing reads corresponding to a number of the first sequence representations 220 having at least a threshold amount of methylated cytosines located. In one or more additional illustrative examples, the classification region metrics 226 can include quantitative measures determined based on a number of nucleic acid molecules that correspond to a number of the first sequence representations 220. In various examples, the classification region metrics 226 can include quantitative measures determined based on a number of first sequence representations 220 having at least a threshold amount of methylated cytosines and a number of second sequence representations 222 that correspond to control regions of a reference sequence. In one or more further illustrative examples, the classification region metrics 226 can include quantitative measures related to a ratio of a number of first sequence representations 220 having at least a threshold amount of methylated cytosines in relation to a number of second sequence representations. In at least some examples, the sequence representations of the second sequence representations 222 used by the computing system 214 to generate quantitative measures included in the classification region metrics 226 can include sequence representations that correspond to positive control regions of a reference sequence.

The classification region metrics 226 can also be determined by performing one or more normalization operations with respect to quantitative measures generated by the computing system 214 using at least one of the first sequence representations 220 and the second sequence representations 222. For example, a logarithm calculation can be performed with respect to quantitative measures generated by the computing system 214 using at least one of the first sequence representations 220 or the second sequence representations 222. Additionally, the classification region metrics 226 can be determined by adding a pseudocount to quantitative measures determined by the computing system 214 using at least one of the first sequence representations 220 or the second sequence representations 222. In one or more illustrative examples, the one or more normalization operations can include determining quantitative measures that correspond to a ratio of first sequence representations 220 for an individual classification region with respect to a number of second sequence representations 222 that correspond to positive control regions of a reference sequence.

In one or more illustrative examples, the computing system 214 can determine a number of the first sequence representations 220 that correspond to individual classification regions of a reference sequence and that have at least a threshold amount of methylated cytosines located in the individual classification regions. In these scenarios, the computing system 214 can determine individual classification region metrics 226 for individual classification regions. In addition, the computing system 214 can determine a number of the second sequence representations 222 that correspond to positive control regions. In at least some examples, the computing system 214 can, for individual classification regions, determine a ratio including a number of first sequence representations 220 that correspond to the individual classification region and that have at least a threshold amount of molecules with a methylated cytosine in the classification region in relation to a total number of the second sequence representations 222 the correspond to positive control regions of a reference sequence. In one or more examples, the computing system 214 can add a value of a pseudocount to the ratio to determine a classification region metric 226 for the individual classification region. The value of the pseudocount can be at least 1, at least 1.2, at least 1.4, at least 1.6, at least 1.8, or at least 2. Further, the computing system 214 can perform a log base 10 operation with respect to the combination of the ratio and the pseudocount to determine a classification region metric 226 for an individual classification region. In at least some illustrative examples, the computing system 214 can determine at least a portion of the classification region metrics according to the following equation:

$$Score of region i = {log}_{10} ( \frac{{x}_{i}}{{x}_{positive\_control}} + pseudocount ) , \qquad ( 1 )$$

where xᵢ is a total number of first sequence representations 220 for an individual classification region, i, having at least a threshold amount of methylated cytosines included in the region, I, and xₚₒₛᵢₜᵢᵥₑ control is a total number of the second sequence representations 222 that correspond to positive control regions of a reference sequence.

At operation 228, the computing system 214 can execute a model to determine an indication of cancer based on the classification region metrics 226. In the illustrative example of FIG. 2, the computing system 214 can execute a model using the classification region metrics 226 to generate model output 230. In one or more examples, the model output 230 can indicate a status of tumor detection 232 or a status of tumor not detected 234 in relation to a sample 204 provided by a subject 206. In one or more additional examples, the computing system 214 can execute a model to determine an estimate of tumor fraction 236 for a sample 204. In one or more further examples, the computing system 214 can execute a model to determine a probability of a tumor being present in a subject 206 that provided a sample 204.

In one or more examples, the model can include a classification model that implements one or more machine learning techniques. In one or more illustrative examples, the model can include a linear regression model. In various examples, the model can be executed to determine a probability of a tumor being present 238 in a subject 206 that provided a sample 204 based on the classification region metrics 226. In one or more illustrative examples, the computing system 214 can execute the model to determine weights for individual classification regions. The weights for individual classification regions can be different. For example, the computing system 214 can determine that a first weight of a first classification region metric 226 for a first classification region is different from a second weight of a second classification region metric 226 for a second classification region. In at least some illustrative examples, a probability of a tumor being present 238 in a subject 206 that provided a sample 204 can be determined by the computing system 214 by executing a model that corresponds to the following equation

$$P ( cancer \vert region scores ) = \frac{1}{1 + {e}^{-} {\sum }_{i} {w}_{i} ( score of region i ) + b} , \qquad ( 2 )$$

where wᵢ is a weight of an individual classification region, the score of the region i is calculated using Equation (1), and b is a slope corresponding to a linear regression model. In at least some examples, the probability of a tumor being present 238 can be used to generate a status of tumor detected 232 or a status of tumor not detected 234. In one or more further illustrative examples, the computing system 214 can analyze the probability of a tumor being present 238 with respect to a threshold probability to determine a status of tumor detected 232 or a status of tumor not detected 234 for a sample 204. The computing system 214 can determine that a sample 204 corresponds to the status of tumor detected 232 in response to determining that a probability of a tumor being present 238 for the sample 204 is at least the threshold probability. Additionally, the computing system 214 can determine that a sample 204 corresponds to the status of tumor not detected 234 in response to determining that a probability of a tumor being present 238 for the sample 204 is less than the threshold probability.

In one or more additional examples, the computing system 214 can execute a model that determines a maximum mutant allele fraction (MAF). In various examples, the computing system 214 can execute a model using the maximum MAF value to determine tumor fraction 236 for a sample 204. In one or more illustrative examples, the computing system 214 can execute a model using the classification region metrics 226 to determine a logit transformed maximum MAF value that can then be used by the computing system 214 to estimate tumor fraction for a sample 204. In various examples, the computing system 214 can analyze maximum MAF values to determine a probability of cancer status 238 in a subject 206 that provided a sample 204. In various examples, a Huber regression (Huber, P. J. 1964. “Robust Estimation of a Location Parameter.” Annals of Mathematical Statistics 35 (1): 73-101) can be performed to determine a maximum MAF value based on the classification region metrics 226.

In various examples, the model output 230 can also include a tumor tissue indication 240. The tumor tissue indication 240 can indicate one or more tissues from which cancer cells that produced genomic material detected in the sample 204 originate. In one or more examples, the tumor tissue indication 240 can correspond to one or more tissues of origin for cancer cells that produced genomic material detected in the sample 204. In these scenarios, the computing system 214 can generate multiple models with individual models corresponding to a given tissue type. The output from individual models can be analyzed to determine additional metrics that indicate a tissue from which cancer cells that produced genomic material detected in one or more samples originate. In at least some examples, the output for the individual models can indicate at least one of tumor fraction 236 or a probability of tumor being present 238. The computing system 214 can analyze the respective model outputs to determine the model having at least one of a greatest value for tumor fraction or a greatest probability of cancer status. The computing system 214 can then generate a tumor tissue indication 240 that corresponds to the model having the greatest value for tumor fraction and/or a greatest probability of cancer status.

For example, samples 204 can be obtained from subjects 206 in which different types of cancer are present. To illustrate, first samples can be obtained from a first group of subjects in which a first classification of cancer is present and second samples can be obtained from a second group of subjects in which a second classification of cancer is present. The sequencing data generated from the first samples can be analyzed by the computing system 214 to generate first metrics that correspond to classification regions for the first classification of cancer and the first metrics can be used to generate a first model that corresponds to the first classification of cancer. Additionally, the sequencing data generated from second samples can be analyzed by the computing system 214 to generate second metrics that correspond to classification regions for the first classification of cancer and the second metrics can be used to generate a second model that corresponds to the second classification of cancer. After the models have been trained, the computing system 214 can analyze sequencing data obtained from one or more additional subjects that were not included in the training subjects to determine classification region metrics for the one or more additional subjects. The classification region metrics can then be analyzed using the different tumor classification models to generate model outputs. The model outputs can be analyzed by the computing system to determine a model having greatest values for the respective model outputs and determine the tumor tissue classification that corresponds to the model.

In one or more additional illustrative implementations, the model output 230 can also indicate methylation status for one or more genomic regions of a reference sequence. For example, the computing system 214 can analyze the classification region metrics 226 to determine a methylation status of one or more promoter regions of a reference sequence. In various examples, the one or more promoter regions can include at least one promoter region that is related to the presence of a tumor in a subject. In one or more illustrative examples, the classification region metrics 226 can indicate a number of sequence representations having at least a threshold amount of methylation with respect to the one or more promoter regions. In these scenarios, the computing system 214 can determine that a promoter region is methylated in response to determining that the number of sequence representations having at least a threshold amount of molecules with a methylated cytosine in the promoter region is greater than a threshold number.

In one or more further illustrative examples, the computing system 214 can combine results from multiple models to determine the model output 230. For example, the computing system 214 can execute models with respect to one or more epigenetic signals, such as methylation of classification regions, to determine one or more first tumor metrics. With regard to methylation, the computing system 214 can execute both a classification model, such as a logistic regression model, that produced an indication of cancer status in a subject providing a sample and an additional model that predicts tumor fraction for a sample. In one or more examples, the epigenetic signals can also correspond to fragment lengths of sequence representations generated from samples. In addition, the computing system 214 can execute one or more additional models with respect to genomic signals to generate further tumor metrics with respect to samples. In various examples, the genomic signals can correspond to the presence of one or more single nucleotide variants (SNVs) and/or the presence of insertions or deletions at one or more genomic regions of a reference sequence. In at least some examples, the computing system can include an integration system that combines tumor metrics generated by executing a number of models with regard to data corresponding to the genomic signals and the epigenetic signals to produce an aggregated tumor metric for a given sample.

In various additional implementations, the computing system 214 can determine methylation status of individual genomic regions. In one or more illustrative examples, the computing system 214 can determine methylation status of one or more promoter regions. In one or more examples, the sequencing data 212 can be analyzed to determine sequence representations that correspond to one or more genomic regions. For example, the sequencing data 212 can be analyzed to determine a number of sequence representations that correspond to one or more promoter regions. In at least some examples, the computing system 214 can determine a number of sequence representations that correspond to individual promoter regions that have at least a threshold amount of methylated cytosines.

For each genomic region and for an individual sample, the computing system 214 can determine a number of sequence representations that correspond to polynucleotide molecules having at least the threshold number of methylated cytosines in the genomic region. The computing system 214 can perform one or more normalization operations using the counts of polynucleotide molecules or sequence reads that correspond to the genomic region and have at least the threshold number of methylated cytosines to generate normalized metrics. To illustrate, the computing system 214 can divide the counts of polynucleotide molecules or reads that correspond to the genomic region and have at least the threshold number of methylated cytosines by the number of molecules or sequencing reads that correspond to a control region, such as a positive control region. In another instance, the computing system 214 can perform the normalized metrics by dividing the counts of polynucleotide molecules or reads that correspond to the genomic region and have at least the threshold number of methylated cytosines by the number of molecules or sequencing reads in a control dataset (i.e., the control dataset includes of tumor not-detected samples) corresponding to the same genomic region and have at least the same threshold number of methylated cytosines.

The normalized metrics can be analyzed with respect to a threshold value. The threshold value can correspond to a given genomic region, such as a given promoter region. In various examples, the threshold value can be different for different promoter regions. In these scenarios, a first promoter region can have a first threshold value and a second promoter region can have a second threshold value. In situations where the normalization metric is at least the threshold value, the computing system 214 can determine that the genomic region has a first methylation status. In scenarios where the normalization metric is less than the threshold value, the computing system 214 can determine that the genomic region has a second methylation status. In one or more illustrative examples, the first methylation status can be labeled as “methylated” and the second methylation status can be labeled as “not methylated.”

The threshold value for a given genomic region can be determined based on training data obtained from samples of individuals in which cancer is not detected. In one or more examples, sequence representations obtained from the training samples can be analyzed to determine a z-score with respect to the number of polynucleotide molecules that correspond to the genomic region and that have at least the threshold amount of methylated cytosines. In one or more illustrative examples, the threshold value for a promoter region that is used to determine the normalization metrics for the promoter region can be derived from the z-score calculated based on the training samples with respect to the promoter region.

Although the illustrative example of FIG. 2 describes that models can be generated to determine a number of indicators with respect to the presence or absence of cancer in a given subject, in at least some additional examples, the sequencing data 212 can be analyzed by the computing system 214 to determine indicators of the presence of cancer without training specific models. In one or more examples, the computing system 214 can determine a tumor fraction value based on sequencing data 212 generated from one or more samples obtained from a single subject in which it is unknown whether or not cancer is present in the subject. In various examples, the plurality of samples obtained from a given subject can be produced by performing a number of titrations on a single sample. In one or more additional examples, a first sample can be obtained from a subject prior to or at onset of at least one administration of a treatment or a procedure related to cancer and one or more second samples can be obtained from the subject after at least one of administration of a treatment or a procedure related to cancer. In one or more illustrative examples, the one or more second samples can be obtained at least one week, at least two weeks, at least three weeks, at least four weeks, at least five weeks, at least six weeks, at least eight weeks, or at least ten weeks after administration of the treatment or procedure. In at least some examples, first sample and the second sample can be derived from at least one of a bodily fluid obtained from the subject or tissue obtained from the subject.

In one or more examples, one or more samples can be obtained from a given subject. The sequencing data 212 generated from the one or more samples can be analyzed by the computing system to determine quantitative measures for a number of classification regions. In one or more examples, the quantitative measures can correspond to an amount of sequence representations that have at least a threshold amount of overlap with one or more classification regions. In one or more additional examples, the quantitative measures can correspond to sequence representations having at least a threshold amount of methylated cytosines in CpG regions having at least a threshold amount of CG content. In various examples, the indication of cancer status in the subject can include tumor fraction. In one or more additional examples, the indication of cancer status in the subject can include mutant allele fraction. In at least some examples, the quantitative measures can correspond to a number of sequencing reads that correspond to a given classification region in relation to a total number of sequencing reads across a plurality of positive control regions. In one or more further examples, the indicators of cancer status can be used to determine an output that corresponds to cancer status or not being present in a given individual in response to analyzing the one or more indicators of cancer status with respect to one or more thresholds. In one or more illustrative examples, tumor fraction determined from one or more samples obtained from a subject can be analyzed with respect to one or more thresholds. In instances where tumor fraction is greater than a threshold level, the computing system 214 can determine that the probability of cancer status in the subject is at least 80%, at least 85%, at least 90%, at least 95%, or at least 99%. Further, in situations where multiple samples are obtained from a subject, first quantitative measures generated from a first sample obtained from the subject can be analyzed with respect to second quantitative measures generated from a second sample obtained from the subject. In at least some examples, differences between the first quantitative measures and the second quantitative measures can be analyzed to determine an indication of treatment response in the subject.

The quantitative measures used to determine an indication of cancer status in a subject can be determined by analyzing quantitative measures of a subset of classification regions. In at least some examples, the subset of classification regions can be different for different subjects. In one or more illustrative examples, values of quantitative measures for a number of classification regions can be analyzed with respect to one another and ranked according to the magnitude of the value of the quantitative measures. In various examples, the classification regions for a given sample can be ranked in descending order from the one or more classification regions having the greatest value of a quantitative measure to the one or more classification region having the least value of the quantitative measure.

In various examples, after ranking the quantitative measures of the classification regions, quantitative measures that correspond to a group of the classification regions can be removed before determining the indication of cancer status in the subject. For example, the group of classification regions that are not used to determine the indication of cancer status in the subject can include the 1% of classification regions having the greatest quantitative measure values, the 2% of classification regions having the greatest quantitative measure values, 3% of classification regions having the greatest quantitative measure values, 4% of classification regions having the greatest quantitative measure values, 5% of classification regions having the greatest quantitative measure values, or the 6% of classification regions having the greatest quantitative measure values. In at least some examples, a number of classification regions having relatively high quantitative measure values can be excluded from the group of classification regions used to determine the indication of cancer status in the subject because, in at least some cases, classification regions corresponding to quantitative measure values at or near the top of the ranked list can have non-tumor origins and/or be related to sequencing artifacts. Thus, by removing the quantitative measures that correspond to these classification regions from the analysis used to determine the indication of cancer status in the subject, the accuracy with which the indication of cancer status in the subject can increase.

In one or more examples, after determining the group of classification regions to be used to determine an indication of cancer status in the subject, a subset of classification regions of the group can then be determined by identifying at least 10 classification regions of the group, at least 25 classification regions of the group, at least 50 classification regions of the group, at least 75 classification regions of the group, at least 100 classification regions of the group, at least 150 classification regions of the group, at least 200 classification regions of the group, at least 250 classification regions of the group, at least 300 classification regions of the group, at least 350 classification regions of the group, at least 400 classification regions of the group, at least 450 classification regions of the group, or at least 500 classification regions of the group having the greatest values for the respective quantitative measure.

In at least some examples, one or more statistical measures, such as at least one of mean, median, or mode, can be applied to the quantitative measures of the subset of the classification regions of the group to generate an initial indication of cancer status in the subject. In various examples, the initial indication of cancer can be modified according to a scaling factor. The scaling factor can be applied to the initial indication of cancer status in the subject because, in at least some scenarios, the positive control regions can have different amounts of methylated CpGs. For example, at least a portion of the positive control regions can have fully methylated CpGs while other positive control regions may not be fully methylated. Additionally, in various situations, some classification regions can correspond to a high value of an indication of cancer status in subjects, such as 90% tumor fraction, 95% tumor fraction, 99% tumor fraction, or 100% tumor fraction, but nucleic acid molecules that correspond to these classification regions may not be fully methylated. To account for these cases, the scaling factor can be applied to the initial indication of cancer status in the subject to provide a more accurate determination of the indication. In one or more illustrative examples, the scaling factor can be determined by analyzing indications of cancer status in subjects determined using one or more techniques described herein in relation to additional data that corresponds to additional indications of cancer status in subjects, such as validation data or other techniques that generate data orthogonal to the indications of tumors being present in subjects described herein.

In various examples, the classification regions used to determine the quantitative measures can correspond to classification regions that correspond to one or more portions of differentially methylated regions. In one or more examples, the differentially methylated regions can include promoter regions that correspond to one or more classifications of cancer. For example, the classification regions can be determined by analyzing a number of sequencing representations across a differentially methylated region. In these scenarios, one or more portions of the differentially methylated regions that overlap with at least at threshold number of sequencing representations can be included in the classification regions. In one or more examples, the quantitative measures of the one or more portions of the differentially methylated regions can be determined based on the molecule count distribution of the differentially methylated region. For example, the quantitative measures can be determined based on the molecule count within one or more peaks of the molecule distribution of the differentially methylated region. To illustrate, in various examples, the distribution of molecules across a differentially methylated region can indicate one or more peaks where greater amounts of molecules overlap with one or more subregions within the differentially methylated region. In various examples, the one or more genomic regions that correspond to the one or more subregions of the differentially methylated regions that correspond to the highest amounts of sequence representations for a sample can be defined as classification regions. In at least some examples, the distribution of sequence representations can have a peak that corresponds to a subregion of the differentially methylated region having a higher number of sequence representations than other subregions of the differentially methylated region. In these scenarios, the subregion can be identified as a classification region. By determining subregions of at least a portion of the differentially methylated regions used to determine the indication of cancer status in the subject, the amount of computing resources and memory resources used to determine the indication of cancer status in the subject can be decreased.

To illustrate, a classification region can include one or more portions of a differentially methylated region in which at least 50% of the sequencing representations obtained from a sample overlap, at least 55% of the sequencing representations obtained from a sample overlap, at least 60% of the sequencing representations obtained from a sample overlap, at least 65% of the sequencing representations obtained from a sample overlap, at least 70% of the sequencing representations obtained from a sample overlap, at least 75% of the sequencing representations obtained from a sample overlap, at least 80% of the sequencing representations obtained from a sample overlap, at least 85% of the sequencing representations obtained from a sample overlap, at least 90% of the sequencing representations obtained from a sample overlap, at least 95% of the sequencing representations obtained from a sample overlap, or at least 99% of the sequencing representations obtained from a sample overlap. In one or more illustrative examples, the one or more portions of the differentially methylated region that comprise a classification region can be contiguous with respect to a reference sequence.

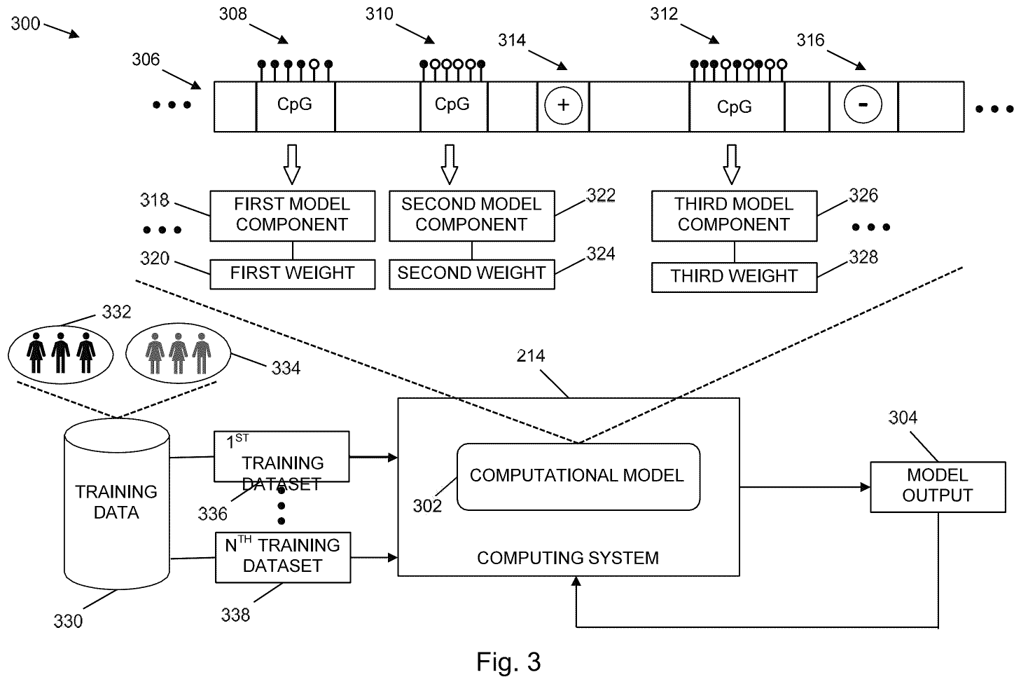

FIG. 3 is a diagrammatic representation of an example framework 300 to train a computational model 302 to determine one or more tumor metrics with respect to a sample, in accordance with one or more implementations. The framework 300 can include the computing system 214. The computing system 214 can execute the computational model 302 to generate one or more model outputs 304. In one or more examples, the computational model 302 can be a machine learning model. The model output 304 can include an indication corresponding to the presence or absence of a tumor in a subject that provided a sample. In one or more illustrative examples, the model output 304 can include a tumor fraction. In one or more additional illustrative examples, the model output 304 can include a probability of cancer status in a subject. In one or more further illustrative examples, the model output can include an indication of cancer status in a subject or an indication of cancer not being present in a subject. In still other illustrative examples, the model output 304 can indicate methylation status of one or more regions of nucleic acid molecules. To illustrate, the computing system 214 can execute the computational model 302 with respect to quantitative measures corresponding to a promoter region to determine an amount of methylation of the promoter region. In other illustrative examples, the model output 304 can include a tumor tissue indication of the sample.

The framework 300 can also include a sequence representation 306. In one or more examples, the sequence representation 306 can be generated based on analyzing nucleic acid molecules that are derived from a sample provided by a subject. The sequence representation 306 can include genomic regions having a number of nucleotides that correspond to a number of regions of interest. For example, the sequence representation 306 can include a sequence of nucleotides that corresponds to a first classification region 308. In addition, the sequence representation 306 can include a sequence of nucleotides that corresponds to a second classification region 310. Further, the sequence representation 306 can include a sequence of nucleotides that corresponds to a third classification region 312. In various examples, the first classification region 308, the second classification region 310, and the third classification region 312 of the sequence representation 306 can have differing amounts of methylated cytosines included in the respective classification regions 308, 310, 312. In one or more additional examples, the sequence representation 306 can include a sequence of nucleotides that corresponds to a positive control region 314 and a sequence of nucleotides that corresponds to a negative control region 316.

The computational model 302 can include a number of components that correspond to individual classification regions. In one or more examples, the components of the computational model 302 can have respective values that correspond to quantitative metrics of the respective classification regions. The quantitative metrics can indicate a number of sequence representations that correspond to the respective classification regions. In one or more examples, the computational model 302 can include a number of weights that are related to the respective components of the computational model 302. For example, the computational model 302 can include a first model component 318 that has a first weight 320. The first model component 318 can correspond to the first classification region 308. In addition, the computational model 302 can include a second model component 322 that has a second weight 324. The second model component 322 can correspond to the second classification region 310. In various examples, at least one of the first weight 320, the second weight 324, or the third weight 328 can be different from at least another one of the first weight 320, the second weight 324, or the third weight 328.

In one or more illustrative examples, a value for the first model component 318, the second model component 322, and the third model component 326 can be determined on a per sample basis. To illustrate, for different samples, the computational model 302 can determine different values for at least one of the first model component 318, the second model component 322, or the third model component 326. In various examples, the computing system 214 can determine first quantitative measures for the first classification region 308 based on sequencing data for a sample. The computing system 214 can execute the computational model 302 to determine a value for the first model component 318 based on the first quantitative measures. Additionally, the computing system 214 can determine second quantitative measures for the second classification region 310 based on sequencing data for the sample. The computing system 214 can execute the computational model 302 to determine a value for the second model component 322 based on the second quantitative measures. Further, the computing system 214 can determine third quantitative measures for the third classification region 312 based on sequencing data for the sample. The computing system 214 can execute the computational model 302 to determine a value for the third model component 326 based on the third quantitative measures. The first quantitative measures, the second quantitative measures, and the third quantitative measures can be determined based on numbers of sequence representations that have at least a threshold amount of methylation in CG regions that correspond to the first classification region 308, the second classification region 310, and the third classification region 312, respectively. In one or more additional illustrative examples, a value for the first weight 320, a value for the second weight 324, and a value for the third weight 328 can be determined on a per sample basis. For example, for different samples, the computational model 302 can determine different values for at least one of the first weight 320, the second weight 324, or the third weight 328.

In one or more examples, the computing system 214 can perform a training process to generate the computational model 302. In various examples, the training process can determine one or more features related to classification region metrics that can be used to determine the model output 304. Additionally, the training process can determine one or more parameters related to classification region metrics that can be used to determine the model output 304. For example, the training process can be used to determine the model components to include in the computational model 302 and the corresponding weights of the model components.

In the illustrative example of FIG. 3, the training process can be performed using training data 330. The training data 330 can include information obtained with respect to at least a first group of subjects 332 and information obtained with respect to at least a second group of subjects 334. In one or more examples, the first group of subjects 332 can include subjects in which a tumor is not detected and the second group of subjects 334 can include subjects in which a tumor is detected. In various examples, the training data 330 can include characteristics related to amounts of methylation of classification regions of the reference sequence 306 for the first group of subjects 332 and the second group of subjects 334. For example, the training data 330 can indicate quantitative measures corresponding to numbers of sequence representations that have at least a threshold level of methylation for the classification regions 308, 310, 312 for the first group of subjects 332 and the second group of subjects 334. The training data 330 can also include weights for model components based on an analysis of sequencing data of the first group of subjects 332 and the second group of subjects 334. In one or more illustrative examples, the training data 330 can include values for the first weight 320, values for the second weight 324, and values for the third weight 328 based on classification region metrics determined from sequencing data obtained from samples provided by the first group of subjects 332 and the second group of subjects 334.

The training data 330 can also include information corresponding to additional characteristics of the first group of subjects 332 and the second group of subjects 334. To illustrate, the training data 330 can include medical records information, medical history information, cancer treatment history information, demographic information, genomics information, one or more combinations thereof, and the like.

In one or more examples, the computing system 214 can train the computational model 302 to determine an indication related to one or more types of cancer status in an individual. Additionally, in various examples, the computational model 302 can comprise multiple different models, such that the computational model 302 is an ensemble model. In these situations, the computing system 214 can perform one or more training processes with respect to individual models of the ensemble model. In one or more illustrative examples, the computational model 302 can include a number of individual models that each correspond to determining model outputs for individual genes or for a specified group of genes. For example, the computation model 302 can include a number of individual models to generate maximum MAF values for individual genes or for a specified groups of genes.

The computing system 214 can obtain a first training dataset 336 up to an Nth training dataset 338 to perform a training process to generate the computational model 302. In one or more examples, the first training dataset 336 can include a first portion of the training data 330 corresponding to the first group of subjects 332 and the second group of subjects 334 that is used to train the computational model 302 and the Nth training dataset 338 can include a second portion of the training data 330 corresponding to the first group of subjects 332 and the second group of subjects 334 as part of a validation process for the computational model 302. In various examples, the computational model 302 can be updated over time and undergo multiple training processes. In these scenarios, the first training dataset 336 can include a portion of the training data 330 for the first group of subjects 332 and the second group of subjects 334 that corresponds to a first period of time and the Nth training dataset 338 can include a portion of the training data 330 for the first group of subjects 332 and the second group of subjects 334 that corresponds to a second period of time.

In one or more examples, during the training process for the computational model 302, the computing system 214 can perform one or more optimization operations. In one or more illustrative examples, the computing system 214 can identify, during the training process for the computational model 302, one or more samples obtained from at least one of the first group of subjects 332 or the second group of subjects 334 that are outliers with respect to samples obtained from other subjects included in at least one of the first group of subjects 332 or the second group of subjects 334. To illustrate, the computing system 214 can determine that model output 304 generated for one or more subjects included in at least one of the first group of subjects 332 or the second group of subjects 334 has at least a threshold amount of difference with the model output 304 generated for one or more additional subjects included in at least one of the first group of subjects 332 or the second group of subjects 334. In one or more examples, the computing system 214 can identify at least one of one or more first subjects 332 or one or more second subjects 334 have model output 304 that is at least one standard deviation, at least 1.5 standard deviations, at least 2 standard deviations, at least 2.5 standard deviations, or at least 3 standard deviations different from a mean model output 304 determined for an additional group of at least one of the first group of subjects 332 or the second group of subjects 334. In various examples, the computing system 214 can apply a penalty to information generated from samples that correspond to subjects that are outliers with respect to information generated from samples that correspond to additional subjects.

In one or more additional examples, one or more optimization processes implemented by the computing system 214 in the training of the computational model 302 can correspond to a number of training cycles and/or a number of iterations for individual training cycles that are performed during the training process.

In one or more illustrative examples, the computing system 214 can perform at least 1000 iterations of a training process to generate the computational model 302, at least 3000 iterations of a training process to generate the computational model 302, at least 5000 iterations of a training process to generate the computational model 302, at least 8000 iterations of a training process to generate the computational model 302, at least 10,000 iterations of a training process to generate the computational model 302, at least 12,000 iterations of a training process to generate the computational model 302, or at least 15,000 iterations of a training process to generate the computational model 302. In various examples, the computing system 214 can end the training process before convergence of a loss function related to the computational model 302. In one or more examples, the number of iterations of the training process to produce the computation model 302 can correspond to a number of iterations of the training process performed before the training process is stopped and before the convergence of the loss function.

In one or more examples, a first stage of the training process implemented by the computing system 214 to generate the computational model 302 can include determining samples included in the training data 330 that include somatic mutations indicative one or more types of cancer in relation to samples included in the training data 330 that do not include somatic mutations indicative of the one or more types of cancer. The computing system 214 can then performing a training process for the computational model 302 using the samples of the training data 330 that include one or more somatic mutations indicative of the one or more types of cancer and using a number of samples obtained from subjects in which a tumor is not detected. In various examples, at least 100 iterations of the first stage of the training process can be performed.

Further, the training process performed by the computing system 214 can include a second stage that includes predicting values of tumor metrics of samples that do not include somatic mutations with respect to the one or more types of cancer. The computing system 214 can the perform at least 100 additional iterations of the second stage of the training process to generate the computational model 302. The second stage of the training process performed by the computing system 214 to generate the computational model 302 can also include training the computational model 302 using portions of the training data 330 corresponding to samples having somatic mutations indicative of the one or more types of cancer, using the predicted values of sample that do not include somatic mutations indicative of the one or more types of cancer, and portions of the training data 330 that correspond to samples obtained from subjects in which a tumor is not detected. In various examples, the second stage of the training process performed by the computing system 214 to generate the computational model 302 can be performed at least 2 additional times, at least 3 additional times, at least 4 additional times, at least 5 additional times, or at least 6 additional times. After the first stage of the training process and the second stage of the training process have been completed, the computing system 214 can perform a validation process for the computational model 302 using information obtained from different samples included in the training data 330.

In one or more illustrative examples, the computing system 214 can perform a training process for multiple computational models 302. In these scenarios, individual computational models 302 trained by the computing system 214 can correspond to different tissue types that are sources of genomic material obtained from subjects included in the training data 330. In one or more examples, the individual computational models 302 trained by the computing system 214 can correspond to different classification of cancer, such as colorectal cancer, lung cancer, pancreatic cancer, bladder cancer, breast cancer, liver cancer, skin cancer, or one or more additional classifications of cancer. In situations where the computing system 214 trains multiple computational models 302 that correspond to different classifications of cancer, the output from individual computational models 302 can be aggregated and analyzed by the computational system 214 to determine a tissue of origin for a subject.

In various examples, the individual computational models 302 that correspond to a given tissue from which genomic material included in samples is derived can have different model components. For example, a first computational model generated by the computing system 214 that corresponds to a first tissue type can have first model components that correspond to a first set of classification regions. In addition, a second computational model generated by the computing system 214 that corresponds to a second tissue type can have second model components that correspond to a second set of classification regions that has at least one classification region different from the first set of classification regions. Additionally, the weights for the individual components of the computational models that correspond to different tissue types can be different. That is, in situations where the first set of classification regions of the first computational model and the second set of classification regions of the second computational model have at least one classification region in common, the weights for the model component that corresponds to the at least one common classification region can be different in relation to the first computational model and the second computational model.

Additionally, one or more additional normalization processes can be performed by the computing system when generating the computational model 302. For example, in at least some scenarios, molecules treated with MBD can be partitioned differently across different samples. In one or more examples, molecules can be partitioned differently across different samples due to differences in the composition of reagents used to treat the molecules with MBD. In one or more additional examples, molecules can be partitioned differently across different samples due to at least one of equipment differences or process conditions used to treat the molecules with MBD.

To illustrate, for one or more first samples, treatment with MBD can cause first molecules having regions with first CG content to be separated into a first partition and second molecules having regions with second CG content to be separate into a second partition. In addition, for one or more second samples, treatment with MBD can cause third molecules having third CG content that is different from the first CG content to be separated into the first partition and fourth molecules having regions with fourth CG content that is different from the second CG content to be separated into the second partition. In various examples, the first molecules can be treated with MBD and separated into the first partition and the second molecules can be treated with MBD and separated into the second partition across a first cutoff range of CG content. Further, the third molecules can be treated with MBD and separated into the first partition and the fourth molecules can be treated with MBD and separated into the second partition across a second cutoff range of CG content that is different from the first cutoff range.

In one or more illustrative examples, the first cutoff range of CG content can include from 3-10 CpGs having methylated cytosines and the second cutoff range can include from 6-14 CpGs having methylated cytosines. In one or more additional illustrative examples, the first cutoff range of CG content can include from 4-9 CpGs having methylated cytosines and the second cutoff range can include from 7-13 CpGs having methylated cytosines. In one or more further illustrative examples, the first cutoff range of CG content can include from 5-8 CpGs having methylated cytosines and the second cutoff range can include from 8-12 CpGs. In still other illustrative examples, the first cutoff range of CG content can include 4-7 CpGs and the second cutoff range can include from 6-10 CpGs. In various examples, the first cutoff range of CG content and the second cutoff range of CG content can be used to determine the threshold amount of methylated cytosines used to determine at least one of training sequencing reads or testing sequencing reads. In at least some examples, the threshold amount of methylated cytosines can include a cutoff number that corresponds to a probability, such as at least about 80%, at least about 85%, at least about 90%, at least about 95%, or at least about 99% of individual molecules treated with MBD being separated into a given partition. In one or more examples, the threshold amount of methylated cytosines can correspond to 5 methylated cytosines, 6 methylated cytosines, 7 methylated cytosines, 8 methylated cytosines, 9 methylated cytosines, 10 methylated cytosines, 11 methylated cytosines, 12 methylated cytosines, 13 methylated cytosines, or 14 methylated cytosines.

In one or more examples, the computing system 214 can generate metrics for individual classification regions based on quantitative measures that are determined using a first number of sequencing reads having a first amount of CG content and a second number of sequencing reads having a second amount of CG content. In at least some examples, the second number of sequencing reads can be used to modify a metric determined using the first number of sequencing reads to account for variations in the separation of molecules treated using MBD for different samples. In various examples, for individual classification regions, a first metric can be determined for a given sample by determining a first quantitative measure that corresponds to a number of molecules having a threshold amount of methylated cytosines and having a first amount of cytosine-guanine content that correspond to the individual classification region. The first metric can also be determined for a given sample by determining a second quantitative measure that corresponds to a number of molecules having a threshold amount of methylated cytosines and having the first amount of cytosine-guanine content that correspond to a plurality of control regions. To illustrate, for an individual classification region, the first metric can be determined using the first quantitative measure for the individual classification region and the second quantitative measure that corresponds to the plurality of control regions.

The normalization process can also include determining, for a given sample, a second metric for the given sample by determining one or more additional quantitative measures based on a number of molecules having at least the threshold amount of methylated cytosines and a second amount of cytosine-guanine content that correspond to the plurality of control regions, where the second amount of cytosine-guanine content is less than the first amount of cytosine-guanine content. In one or more examples, the second metric can be determined using the third quantitative measure and the second quantitative measure. In at least some examples, the second metric can be determined for a given sample by determining a ratio of the one or more additional quantitative measures with respect to the second quantitative measure. In one or more additional examples, the second metric can be determined for a given sample by determining the logarithm, such as the logarithm according to base 10, of a ratio of the one or more additional quantitative measures with respect to the second quantitative measure.

In one or more illustrative examples, the second metric for a given sample can include a combination of values, where individual values correspond to an additional quantitative measure based on a number of molecules having at least a threshold amount of methylated cytosines and a given number of CpGs for the plurality of control regions and the second quantitative measure. For example, a first additional quantitative measure can be determined based on a first number of molecules having at least the threshold amount of methylated cytosines in control regions having a first number of CpGs, such as 6, and a second additional quantitative measure can be determined based on a second number of molecules having at least the threshold amount of methylated cytosines in control regions having a second number of CpGs, such as 7. In at least some examples, more additional quantitative measures can be determined based on additional numbers of molecules having the threshold amount of methylated cytosines in control regions having additional numbers of CpGs, such as 8 CpGs, 9, CpGs, 10 CpGs, and the like up to an upper threshold of CpGs, such as 12 CpGs, 13 CpGs, or 14 CpGs. Ratios determined using the additional quantitative measures with respect to the second quantitative measures can be determined and summed to determine the second metric.

In various examples, a correlation factor can also be determined for individual classification regions in relation to different amounts of CpGs that can be used to determine the second metric. In one or more examples, the correlation factor can be modify the individual additional quantitative measures and then the modified individual additional quantitative measures can be aggregated to determine the second metric. In one or more additional examples, the first metric and the second metric can be combined to determine a normalized metric that corresponds to a given classification region. In one or more illustrative examples, the second metric can be subtracted from the first metric to determine the normalized metric.

In one or more additional illustrative examples, the correlation factor for a given classification region can be determined for each of a plurality of different amounts of cytosine-guanine content, such as a first correlation factor for 6 CpGs, a second correlation factor for 7 CpGs, a third correlation factor for 8 CpGs, and so forth up to a threshold amount of CG content. In at least some examples, the correlation factor can be determined by analyzing training data using one or more linear regression techniques. For example, the training data 330 can be fit to a linear regression model for individual classification regions to determine the correlation factor. In various examples, the fitting of at least a portion of the training data 330 to the linear regression model can be performed by aggregating the additional quantitative measures for a given classification region across a range of CG content, such a 6 CpGs, 7 CpGs, up to a threshold number of CpGs, and determining a mean quantitative measure.

In one or more examples, the normalized metrics can reduce variation of quantitative measures determined for individual samples. In at least some examples, the reduction in variation can result in increased accuracy of model outputs 304 in relation to at least some model outputs 304 determined without implementing the additional normalization process to determine the normalized metric.

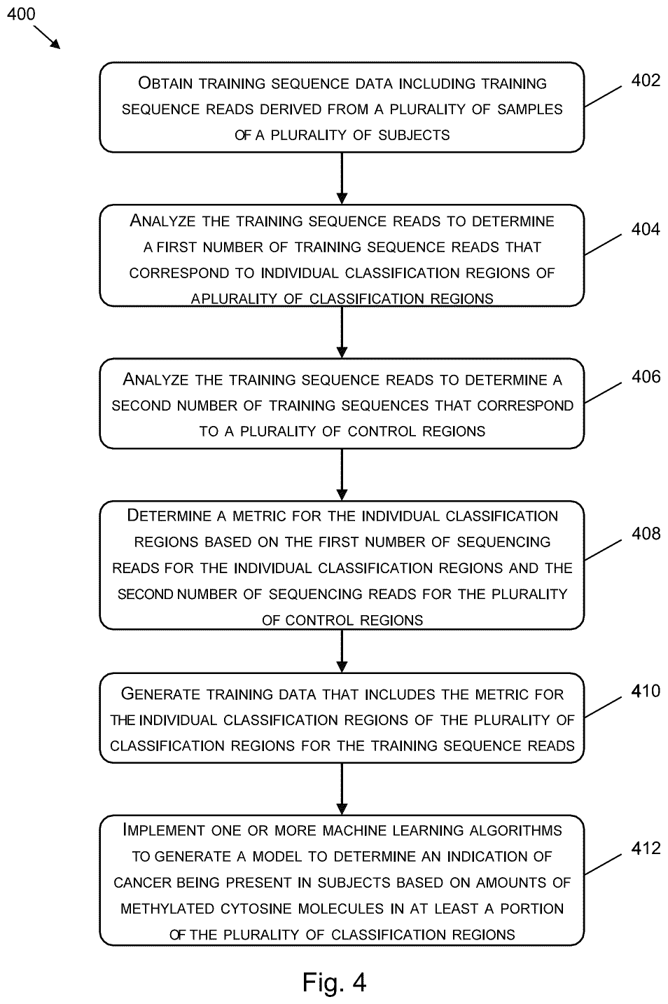

FIG. 4 is a flowchart of an example method 400 to determine tumor metrics in a subject based on levels of methylation of classification regions, according to one or more implementations. At operation 402, the method 400 can include obtaining training sequence data including training sequencing reads derived from a plurality of samples of a plurality of subjects. Individual training sequencing reads can include a nucleotide sequence corresponding to a fragment of a nucleic acid included in a sample of the plurality of samples. Individual training sequencing reads can have a threshold amount of molecules with a methylated cytosine included in regions of the nucleotide sequence having at least a threshold cytosine-guanine content. In one or more illustrative examples, the plurality of samples can include cell-free nucleic acids. In one or more examples, methylated cytosines can be determined using at least one of sodium bisulfite conversion and sequencing, Tet-assisted bisulfite sequencing (TAB-Seq), differential enzymatic cleavage, treatment with MSRE and/or MDRE, or MBD partitioning. In one or more additional examples, methylated cytosines can be determined using one or more single molecule sequencing methods, such as nanopore DNA sequencing or those described in Eid, J., et al. (2009) Real-time DNA sequencing from single polymerase molecules. Science, 323(5910), 133-138.

In one or more examples, the training process can include obtaining, by the computing system, testing sequence data from an additional subject that is not included in the plurality of subjects. The testing sequence data can include testing sequencing reads derived from a sample of the additional subject. Individual testing sequencing reads can include a nucleotide sequence corresponding to a fragment of a nucleic acid included in the additional sample. Additionally, individual testing sequencing reads can have at least the threshold amount of molecules with a methylated cytosine included in regions of the nucleotide sequence having at least the threshold cytosine-guanine content. Based on the additional sequence data, a model can be executed to determine the indication of cancer status in the additional subject. The testing sequencing reads can then be analyzed to determine a first quantitative measure derived from the testing sequencing reads that correspond to the individual classification regions of the plurality of classification regions. Further, the testing sequencing reads can be analyzed to determine a second quantitative measure derived from the testing sequencing reads that correspond to the individual control regions the plurality of control regions. The metric can then be determined for the individual classification regions based on the first quantitative measure for the individual classification regions and the second quantitative measure for the plurality of control regions. Subsequently, an input vector can be generated that includes the metrics for the individual classification regions. The model can use the input vector to determine the indication of cancer status in the additional subject.

In situations where the model is trained to determine an estimate of tumor fraction, the training sequencing reads can comprise a first portion of the training sequence data and a second portion of the training sequence data includes additional training sequencing reads that are different from the training sequencing reads. In these scenarios, at least one of the first portion of the training sequence data or the second portion of the training sequence data can be analyzed to determine an individual frequency of a plurality of variants present in individual samples of the plurality of samples. With respect to individual samples, a variant of the plurality of variants having a maximum frequency can then be determined that corresponds to the individual frequency having a greatest value among individual frequencies derived from an individual sample. In one or more illustrative examples, the maximum mutant allele frequency can be determined for individual samples. In various examples, individual measures of tumor fraction for the individual samples can then be determined based on the greatest value of the individual frequencies derived from the individual sample.

In at least some examples, the training process for the model can include one or more optimization operations. For example, the training process can include determining one or more additional weights of individual samples included in the training data based on the indication of cancer for the individual samples being within a threshold confidence level. In response to determining that the indication of cancer for an individual sample is outside of the threshold confidence level a penalty to can be applied to the individual sample during the training process.

The one or more training optimization operations can also include performing, using the one or more machine learning algorithms, one or more first iterations of the training process for the model using a portion of the training data. In addition, first output data for the model can be generated based on the one or more first iterations of the training process. The first output data can correspond to one or more first additional indications of cancer status in first individual subjects of the plurality of subjects and the first individual subjects can correspond to the portion of the training data. Further, the training process can include combining the first output data and the training data to produce additional training data and performing one or more second iterations of the training process for the model using a portion of the additional training data. Second output data can then be generated for the model based on the one or more second iterations of the training process. The second output data can indicate one or more second additional indications of cancer status in second individual subjects of the plurality of subjects where the second individual subjects corresponding to the portion of the additional training data. In one or more illustrative examples, the weights for the individual classification regions of the plurality of classification regions can be determined based on the first output data and the second output data.

Further, the training process can include determining that a number of indications of cancer are present that were determined during one or more iterations of the training process and have at least a threshold value for one or more samples included in the training data. In these scenarios, modifications to one or more weights of the model are not modified or are modified by a minimal amount. Additionally, an additional number of indications of cancer status can be determined that were determined during the one or more iterations of the training process and are less than the threshold value for one or more additional samples included in the training data. In these scenarios, modifications to one or more additional weights of the model can be determined and the one or more additional weights are modified by more than the minimal amount.

In addition, at operation 404, the process 400 can include analyzing the training sequencing reads to determine a first quantitative measure derived from the training sequencing reads that corresponds to individual classification regions of a plurality of classification regions. In one or more examples, the first quantitative measure can be determined based on the number of training sequencing reads. In one or more additional examples, the first quantitative measure can be determined based on a number of polynucleotide molecules that correspond to the training sequencing reads. At least a portion of the individual classification regions of the plurality of classification regions corresponding to genomic regions of a reference genome that have the threshold amount of molecules with a methylated cytosine in subjects in which cancer is detected and that have at least the threshold cytosine-guanine content. In various examples, the plurality of classification regions can correspond to genomic regions in which at least one mutation occurs in patients in which cancer is detected. Additionally, the plurality of classification regions can correspond to a first plurality of classification regions for a first cancer type and the model can be generated for a second cancer type based on a second plurality of classification regions that are different from the first plurality of classification regions.

At operation 406, the process 400 can include analyzing the training sequencing reads to determine a second quantitative measure derived from the training sequencing reads that correspond to a plurality of control regions. In one or more examples, the second quantitative measure can be determined based on the number of training sequencing reads. In one or more additional examples, the second quantitative measure can be determined based on a number of polynucleotide molecules that correspond to the training sequencing reads. Individual control regions of the plurality of control regions corresponding to additional genomic regions of the reference genome that have at least the threshold cytosine-guanine content. Additionally, the individual control regions can have at least the threshold amount of molecules with a methylated cytosine in subjects in which cancer is detected and in additional subjects in which cancer is not detected

Further, at operation 408, the process 400 can include determining a metric for the individual classification regions of the plurality of classification regions based on the first quantitative measure for the individual classification regions and the second quantitative measure for the plurality of control regions. In one or more examples, the metric for the individual classification regions is determined based on a scaling factor and an error correction factor. In one or more illustrative examples, the scaling factor can include a logarithmic function and the error correction factor can include a pseudocount.

At operation 410, the process 400 can include generating, by the computing device, training data that includes the metric for the individual classification regions of the plurality of classification regions for the training sequence reads. In implementations where the indication of cancer is tumor fraction, the training data can include the individual measures of tumor fraction for the individual samples of the plurality of samples and the model can be executed with respect to individual measures of tumor fraction for the individual samples of the plurality of samples.

The process 400 can also include, at operation 412, implementing, using the training data, one or more machine learning algorithms to generate a model to determine an indication of cancer status in subjects based on amounts of molecules with methylated cytosines in at least a portion of the plurality of classification regions. The model can determine weights for individual classification regions of the plurality of classification regions and at least a portion of the weights of the individual classification regions can be different from one another. In various examples, the one or more machine learning algorithms can include one or more classification algorithms and the indication of cancer status corresponds to a probability of cancer status in the additional subject. In one or more additional examples, the one or more machine learning algorithms include one or more regression algorithms and the indicator corresponds to an estimate of tumor fraction of the additional sample. In one or more illustrative examples, a limit of detection for the model to determine tumor fraction of samples can be no greater than 0.01% at 95% confidence levels, no greater than 0.05% at 95% confidence levels, no greater than 0.1% at 95% confidence levels, no greater than 0.15% at 95% confidence levels, no greater than 0.2% at 95% confidence levels, no greater than 0.25% at 95% confidence levels, or no greater than 0.3% at 95% confidence levels.

In various examples, the sequence reads provided to the model during the training process or after the training process have at least a threshold amount of methylated cytosines in classification regions. The sequence reads that satisfy the methylation levels can be produced, at least in party, using one or more molecule separation processes. The molecule separation processes can include combining a plurality of nucleic acids derived from at least one of blood or tissue of a subject with a solution including an amount of methyl binding domain (MBD) proteins to produce a nucleic acid-MBD protein solution. A plurality of washes can then be performed of the nucleic acid-MBD protein solution with a salt solution to produce a number of nucleic acid fractions. Individual nucleic acid fractions can have a threshold number of molecules with a methylated cytosine in regions of the plurality of nucleic acids having at least the threshold cytosine-guanine content. In one or more illustrative examples, a wash of the plurality of washes can be performed with a solution having a concentration of sodium chloride (NaCl) and can produce a nucleic acid fraction of the number of nucleic acid fractions having a range of binding strengths to MBD proteins.

In one or more examples, a first nucleic acid fraction can be determined is associated with a first partition of a plurality of partitions of nucleic acids. The first partition corresponding to a first range of binding strengths to MBD proteins. Further, a first molecular barcode can be attached to nucleic acids of the first nucleic acid fraction. The first molecular barcode can be associated with the first partition. In addition, a second nucleic acid fraction can be determined that is associated with a second partition of the plurality of partitions of nucleic acids. The second partition can correspond to a second range of binding strengths to MBD proteins different from the first range of binding strengths to MBD proteins. A second molecular barcode can be attached to nucleic acids of the second nucleic acid fraction. The second molecular barcode being associated with the second partition.

In one or more additional examples, at least a portion of the number of nucleic acid fractions can be combined with an amount of restriction enzyme that cleaves molecules with one or more unmethylated cytosines to produce at least a portion of the plurality of samples used to produce the sequencing reads. In these scenarios, the threshold amount of molecules with a methylated cytosine corresponds to a minimum frequency of molecules with a methylated cytosine within a region having at least the threshold cytosine-guanine content. In one or more further examples, at least a portion of the number of nucleic acid fractions are combined with an amount of a restriction enzyme that cleaves molecules with a methylated cytosine to produce at least a portion of the plurality of samples used to produce the sequencing reads. In these situations, the threshold amount of molecules with a methylated cytosine corresponds to a maximum frequency of molecules with a methylated cytosine within a region having at least the threshold cytosine-guanine content.

### Adapter Ligation or Addition

In some embodiments, adapters are added to the DNA. This may be done concurrently with an amplification procedure, e.g., by providing the adapters in a 5′ portion of a primer (where PCR is used, this can be referred to as library prep-PCR or LP-PCR). In some embodiments, adapters are added by other approaches, such as ligation. In some such methods, prior to partitioning or prior to capturing, first adapters are added to the nucleic acids by ligation to the 3′ ends thereof, which may include ligation to single-stranded DNA. The adapter can be used as a priming site for second-strand synthesis, e.g., using a universal primer and a DNA polymerase. A second adapter can then be ligated to at least the 3′ end of the second strand of the now double-stranded molecule. In some embodiments, the first adapter includes an affinity tag, such as biotin, and nucleic acid ligated to the first adapter is bound to a solid support (e.g., bead), which may comprise a binding partner for the affinity tag such as streptavidin. For further discussion of a related procedure, see Gansauge et al., Nature Protocols 8:737-748 (2013). Commercial kits for sequencing library preparation compatible with single-stranded nucleic acids are available, e.g., the Accel-NGS® Methyl-Seq DNA Library Kit from Swift Biosciences. In some embodiments, after adapter ligation, nucleic acids are amplified.

Preferably, the adapters include different tags of sufficient numbers that the number of combinations of tags results in a low probability e.g., 95, 99 or 99.9% of two nucleic acids with the same start and stop points receiving the same combination of tags. Adapters, whether bearing the same or different tags, can include the same or different primer binding sites, but preferably adapters include the same primer binding site.

In some embodiments, following attachment of adapters, the nucleic acids are subject to amplification. The amplification can use, e.g., universal primers that recognize primer binding sites in the adapters.

In some embodiments, following attachment of adapters, the DNA is partitioned, comprising contacting the DNA with an agent that preferentially binds to nucleic acids bearing an epigenetic modification. The nucleic acids are partitioned into at least two subsamples differing in the extent to which the nucleic acids bear the modification from binding to the agents. For example, if the agent has affinity for nucleic acids bearing the modification, nucleic acids overrepresented in the modification (compared with median representation in the population) preferentially bind to the agent, whereas nucleic acids underrepresented for the modification do not bind or are more easily eluted from the agent. The nucleic acids can then be amplified from primers binding to the primer binding sites within the adapters. Partitioning may be performed instead before adapter attachment, in which case the adapters may comprise differential tags that include a component that identifies which partition a molecule occurred in. [0214] In some embodiments, the nucleic acids are linked at both ends to Y-shaped adapters including primer binding sites and tags. The molecules are amplified

### Tagging

“Tagging” DNA molecules is a procedure in which a tag is attached to or associated with the DNA molecules. Tags can be molecules, such as nucleic acids, containing information that indicates a feature of the molecule with which the tag is associated. For example, molecules can bear a sample tag (which distinguishes molecules in one sample from those in a different sample) or a molecular tag/molecular barcode/barcode (which distinguishes different molecules from one another (in both unique and non-unique tagging scenarios). For methods that involve a partitioning step, a partition tag (which distinguishes molecules in one partition from those in a different partition) may be included. In some embodiments, adapters added to DNA molecules comprise tags. In certain embodiments, a tag can comprise one or a combination of barcodes. As used herein, the term “barcode” refers to a nucleic acid molecule having a particular nucleotide sequence, or to the nucleotide sequence, itself, depending on context. A barcode can have, for example, between 10 and 100 nucleotides. A collection of barcodes can have degenerate sequences or can have sequences having a certain hamming distance, as desired for the specific purpose. So, for example, a molecular barcode can be comprised of one barcode or a combination of two barcodes, each attached to different ends of a molecule. Additionally or alternatively, for different partitions and/or samples, different sets of molecular barcodes, or molecular tags can be used such that the barcodes serve as a molecular tag through their individual sequences and also serve to identify the partition and/or sample to which they correspond based the set of which they are a member.

In some embodiments, two or more partitions, e.g., each partition, is/are differentially tagged. Tags can be used to label the individual polynucleotide population partitions so as to correlate the tag (or tags) with a specific partition. Alternatively, tags can be used in embodiments that do not employ a partitioning step. In some embodiments, a single tag can be used to label a specific partition. In some embodiments, multiple different tags can be used to label a specific partition. In embodiments employing multiple different tags to label a specific partition, the set of tags used to label one partition can be readily differentiated for the set of tags used to label other partitions. In some embodiments, the tags may have additional functions, for example the tags can be used to index sample sources or used as unique molecular identifiers (which can be used to improve the quality of sequencing data by differentiating sequencing errors from mutations, for example as in Kinde et al., Proc Nat'l Acad Sci USA 108: 9530-9535 (2011), Kou et al., PLoS ONE, 11: e0146638 (2016)) or used as non-unique molecule identifiers, for example as described in U.S. Pat. No. 9,598,731. Similarly, in some embodiments, the tags may have additional functions, for example the tags can be used to index sample sources or used as non-unique molecular identifiers (which can be used to improve the quality of sequencing data by differentiating sequencing errors from mutations). [0217] In some embodiments, partition tagging includes tagging molecules in each partition with a partition tag. After re-combining partitions (e.g., to reduce the number of sequencing runs needed and avoid unnecessary cost) and sequencing molecules, the partition tags identify the source partition. In some embodiments, the partition tags can serve as identifiers of the source partition and the molecule, i.e., different partitions are tagged with different sets of molecular tags, e.g., comprised of a pair of barcodes. In this way, the one or more molecular barcodes attached to the molecule indicates the source partition as well as being useful to distinguish molecules within a partition. For example, a first set of 35 barcodes can be used to tag molecules in a first partition, while a second set of 35 barcodes can be used tag molecules in a second partition.

In some embodiments, after partitioning and tagging with partition tags, the molecules may be pooled for sequencing in a single run. In some embodiments, a sample tag is added to the molecules, e.g., in a step subsequent to addition of partition tags and pooling. Sample tags can facilitate pooling material generated from multiple samples for sequencing in a single sequencing run.

Alternatively, in some embodiments, partition tags may be correlated to the sample as well as the partition. As a simple example, a first tag can indicate a first partition of a first sample; a second tag can indicate a second partition of the first sample; a third tag can indicate a first partition of a second sample; and a fourth tag can indicate a second partition of the second sample.

While tags may be attached to molecules already partitioned based on one or more characteristics, the final tagged molecules in the library may no longer possess that characteristic. For example, while single stranded DNA molecules may be partitioned and tagged, the final tagged molecules in the library are likely to be double stranded. Similarly, while DNA may be subject to partition based on different levels of methylation, in the final library, tagged molecules derived from these molecules are likely to be unmethylated. Accordingly, the tag attached to molecule in the library typically indicates the characteristic of the “parent molecule” from which the ultimate tagged molecule is derived, not necessarily to characteristic of the tagged molecule, itself.

As an example, barcodes 1, 2, 3, 4, etc. are used to tag and label molecules in the first partition; barcodes A, B, C, D, etc. are used to tag and label molecules in the second partition; and barcodes a, b, c, d, etc. are used to tag and label molecules in the third partition. Differentially tagged partitions can be pooled prior to sequencing. Differentially tagged partitions can be separately sequenced or sequenced together concurrently, e.g., in the same flow cell of an Illumina sequencer.

After sequencing, analysis of reads can be performed on a partition-by-partition level, as well as a whole DNA population level. Tags are used to sort reads from different partitions. Analysis can include in silico analysis to determine genetic and epigenetic variation (one or more of methylation, chromatin structure, etc.) using sequence information, genomic coordinates length, coverage, and/or copy number. In some embodiments, higher coverage can correlate with higher nucleosome occupancy in genomic region while lower coverage can correlate with lower nucleosome occupancy or a nucleosome depleted region (NDR).

### Enriching/Capturing Step; Amplification

Methods disclosed herein can comprise capturing DNA, such as cfDNA target regions. In some embodiments, the capturing includes contacting the DNA with probes (e.g., oligonucleotides) specific for the target regions. Enrichment or capture may be performed on any sample or subsample described herein using any suitable approach known in the art.

In some embodiments, enrichment or capture is performed after attachment of adapters to sample molecules. In some embodiments, enrichment or capture is performed after a partitioning step. In some embodiments, enrichment or capture is performed after an amplification step. In some embodiments, sample molecules are partitioned, then adapters are attached, then sample molecules are amplified, and then the amplified molecules are subjected to enrichment or capture. The enriched or captured molecules may then be subjected to another amplification and then sequenced.

In some embodiments, the probes specific for the target regions comprise a capture moiety that facilitates the enrichment or capture of the DNA hybridized to the probes. In some embodiments, the capture moiety is biotin. In some such embodiments, streptavidin attached to a solid support, such as magnetic beads, is used to bind to the biotin. Nonspecifically bound DNA that does not comprise a target region is washed away from the captured DNA. In some embodiments, DNA is then dissociated from the probes and eluted from the solid support using salt washes or buffers comprising another DNA denaturing agent. In some embodiments, the probes are also eluted from the solid support by, e.g., disrupting the biotin-streptavidin interaction. In some embodiments, captured DNA is amplified following elution from the solid support. In some such embodiments, DNA comprising adapters is amplified using PCR primers that anneal to the adapters. In some embodiments, captured DNA is amplified while attached to the solid support. In some such embodiments, the amplification includes use of a PCR primer that anneals to a sequence within an adapter and a PCR primer that anneals to a sequence within a probe annealed to the target region of the DNA.

In some embodiments, the methods herein comprise enriching for or capturing DNA comprising epigenetic and/or sequence-variable target regions. Such regions may be captured from an aliquot of a sample (e.g., a sample that has undergone attachment of adapters and amplification), while the step of partitioning the DNA with an agent that recognizes a modified cytosine, such as methyl cytosine, is performed on a separate aliquot of the sample. Enriching for or capturing DNA comprising epigenetic and/or sequence-variable target regions may comprise contacting the DNA with a first or second set of target-specific probes. Such target-specific probes may have any of the features described herein for sets of target-specific probes, including but not limited to in the embodiments set forth above and the sections relating to probes below. Capturing may be performed on one or more subsamples prepared during methods disclosed herein. In some embodiments, DNA is captured from the first subsample or the second subsample, e.g., the first subsample and the second subsample. In some embodiments, the subsamples are differentially tagged (e.g., as described herein) and then pooled before undergoing capture. Exemplary methods for capturing DNA comprising epigenetic and/or sequence-variable target regions can be found in, e.g., WO 2020/160414, which is hereby incorporated by reference.

The capturing step may be performed using conditions suitable for specific nucleic acid hybridization, which generally depend to some extent on features of the probes such as length, base composition, etc. Those skilled in the art will be familiar with appropriate conditions given general knowledge in the art regarding nucleic acid hybridization. In some embodiments, complexes of target-specific probes and DNA are formed.

In some embodiments, methods described herein comprise capturing a plurality of sets of target regions of cfDNA obtained from a subject. The target regions may comprise differences depending on whether they originated from a tumor or from healthy cells or from a certain cell type. The capturing step produces a captured set of cfDNA molecules. In some embodiments, cfDNA molecules corresponding to a sequence-variable target region set are captured at a greater capture yield in the captured set of cfDNA molecules than cfDNA molecules corresponding to an epigenetic target region set. In some embodiments, a method described herein includes contacting cfDNA obtained from a subject with a set of target-specific probes, wherein the set of target-specific probes is configured to capture cfDNA corresponding to the sequence-variable target region set at a greater capture yield than cfDNA corresponding to the epigenetic target region set. For additional discussion of capturing steps, capture yields, and related aspects, see W02020/160414, which is incorporated herein by reference for all purposes.

It can be beneficial to capture cfDNA corresponding to the sequence-variable target region set at a greater capture yield than cfDNA corresponding to the epigenetic target region set because a greater depth of sequencing may be necessary to analyze the sequence-variable target regions with sufficient confidence or accuracy than may be necessary to analyze the epigenetic target regions. The volume of data needed to determine fragmentation patterns (e.g., to test for perturbation of transcription start sites or CTCF binding sites) or fragment abundance (e.g., in hypermethylated and hypomethylated partitions) is generally less than the volume of data needed to determine the presence or absence of cancer-related sequence mutations. Capturing the target region sets at different yields can facilitate sequencing the target regions to different depths of sequencing in the same sequencing run (e.g., using a pooled mixture and/or in the same sequencing cell).

In some embodiments, the DNA is amplified. In some embodiments, amplification is performed before the capturing step. In some embodiments, amplification is performed after the capturing step. In some embodiments, amplification is performed before and after the capturing step. In various embodiments, the methods further comprise sequencing the captured DNA, e.g., to different degrees of sequencing depth for the epigenetic and sequence-variable target region sets, consistent with the discussion herein.

In some embodiments, a capturing step is performed with probes for a sequence-variable target region set and probes for an epigenetic target region set in the same vessel at the same time, e.g., the probes for the sequence-variable and epigenetic target region sets are in the same composition. This approach provides a relatively streamlined workflow. In some embodiments, the concentration of the probes for the sequence-variable target region set is greater that the concentration of the probes for the epigenetic target region set.

Alternatively, a capturing step is performed with a sequence-variable target region probe set in a first vessel and with an epigenetic target region probe set in a second vessel, or a contacting step is performed with a sequence-variable target region probe set at a first time and a first vessel and an epigenetic target region probe set at a second time before or after the first time. This approach allows for preparation of separate first and second compositions comprising captured DNA corresponding to a sequence-variable target region set and captured DNA corresponding to an epigenetic target region set. The compositions can be processed separately as desired (e.g., to partition based on methylation as described herein) and pooled in appropriate proportions to provide material for further processing and analysis such as sequencing.

In some embodiments, adapters are included in the DNA as described herein. In some embodiments, tags, which may be or include barcodes, are included in the DNA. In some embodiments, such tags are included in adapters. Tags can facilitate identification of the origin of a nucleic acid. For example, barcodes can be used to allow the origin (e.g., subject) whence the DNA came to be identified following pooling of a plurality of samples for parallel sequencing. This may be done concurrently with an amplification procedure, e.g., by providing the barcodes in a 5′ portion of a primer, e.g., as described herein. In some embodiments, adapters and tags/barcodes are provided by the same primer or primer set. For example, the barcode may be located 3′ of the adapter and 5′ of the target-hybridizing portion of the primer. Alternatively, barcodes can be added by other approaches, such as ligation, optionally together with adapters in the same ligation substrate.

Additional details regarding amplification, tags, and barcodes are discussed herein, which can be combined to the extent practicable with any of these embodiments.

Procedures that Affect a First Nucleobase in the DNA Differently from a Second Nucleobase in the DNA

In some embodiments, methods disclosed herein comprise a step of subjecting DNA to a procedure that affects a first nucleobase in the DNA differently from a second nucleobase in the DNA, wherein the first nucleobase is a modified or unmodified nucleobase, the second nucleobase is a modified or unmodified nucleobase different from the first nucleobase, and the first nucleobase and the second nucleobase have the same base pairing specificity. In some embodiments, the procedure chemically converts the first or second nucleobase such that the base pairing specificity of the converted nucleobase is altered. In some embodiments, if the first nucleobase is a modified or unmodified adenine, then the second nucleobase is a modified or unmodified adenine; if the first nucleobase is a modified or unmodified cytosine, then the second nucleobase is a modified or unmodified cytosine; if the first nucleobase is a modified or unmodified guanine, then the second nucleobase is a modified or unmodified guanine; and if the first nucleobase is a modified or unmodified thymine, then the second nucleobase is a modified or unmodified thymine (where modified and unmodified uracil are encompassed within modified thymine for the purpose of this step).

In some embodiments, the first nucleobase is a modified or unmodified cytosine, then the second nucleobase is a modified or unmodified cytosine. For example, first nucleobase may comprise unmodified cytosine (C) and the second nucleobase may comprise one or more of 5-methylcytosine (mC) and 5-hydroxymethylcytosine (hmC). Alternatively, the second nucleobase may comprise C and the first nucleobase may comprise one or more of mC and hmC. Other combinations are also possible, as indicated, e.g., in the Summary above and the following discussion, such as where one of the first and second nucleobases includes mC and the other includes hmC.

In some embodiments, the procedure that affects a first nucleobase in the DNA differently from a second nucleobase in the DNA includes bisulfite conversion. Treatment with bisulfite converts unmodified cytosine and certain modified cytosines (e.g. 5-formyl cytosine (fC) or 5-carboxylcytosine (caC)) to uracil whereas other modified cytosines (e.g., 5-methylcytosine, 5-hydroxylmethylcystosine) are not converted. Performing bisulfite conversion can facilitate identifying positions containing mC or hmC using the sequence reads. For an exemplary description of bisulfite conversion, see, e.g., Moss et al., Nat Commun. 2018; 9: 5068. [0238] In some embodiments, the procedure that affects a first nucleobase in the DNA differently from a second nucleobase in the DNA includes oxidative bisulfite (Ox-BS) conversion. Performing Ox-BS conversion can facilitate identifying positions containing mC using the sequence reads. For an exemplary description of oxidative bisulfite conversion, see, e.g., Booth et al., Science 2012; 336: 934-937.

In some embodiments, the procedure that affects a first nucleobase in the DNA differently from a second nucleobase in the DNA includes Tet-assisted bisulfite (TAB) conversion. For example, as described in Yu et al., Cell 2012; 149: 1368-80, b-glucosyl transferase can be used to protect hmC (forming 5-glucosylhydroxymethylcytosine (ghmC)), then a TET protein such as mTetl can be used to convert mC to caC, and then bisulfite treatment can be used to convert C and caC to U while ghmC remains unaffected. Thus, when TAB conversion is used, the first nucleobase includes one or more of unmodified cytosine, fC, caC, mC, or other cytosine forms affected by bisulfite, and the second nucleobase includes hmC. Performing TAB conversion can facilitate identifying positions containing hmC using the sequence reads.

In some embodiments, the procedure that affects a first nucleobase in the DNA differently from a second nucleobase in the DNA includes Tet-assisted conversion with a substituted borane reducing agent, optionally wherein the substituted borane reducing agent is 2-picoline borane, borane pyridine, tert-butylamine borane, or ammonia borane. See, e.g., Liu et al., Nature Biotechnology 2019; 37:424-429 (e.g., at Supplementary FIG. 1 and Supplementary Note 7). Performing TAP conversion can facilitate identifying positions containing unmodified C using the sequence reads. This procedure encompasses Tet-assisted pyridine borane sequencing (TAPS), described in further detail in Liu et al. 2019, supra.

Alternatively, protection of hmC (e.g., using bOT) can be combined with Tet-assisted conversion with a substituted borane reducing agent. Performing such TAPSP conversion can facilitate distinguishing positions containing unmodified C or hmC on the one hand from positions containing mC using the sequence reads. For an exemplary description of this type of conversion, see, e.g., Liu et al., Nature Biotechnology 2019; 37:424-429.

In some embodiments, the procedure that affects a first nucleobase in the DNA differently from a second nucleobase in the DNA includes APOBEC-coupled epigenetic (ACE) conversion. Performing ACE conversion can facilitate distinguishing positions containing hmC from positions containing mC or unmodified C using the sequence reads. For an exemplary description of ACE conversion, see, e.g., Schutsky et al., Nature Biotechnology 2018; 36: 1083-1090.

In some embodiments, procedure that affects a first nucleobase in the DNA differently from a second nucleobase in the DNA includes enzymatic conversion of the first nucleobase, e.g., as in EM-Seq. See, e.g., Vaisvila R, et al. (2019) EM-seq: Detection of DNA methylation at single base resolution from picograms of DNA. bioRxiv; DOI [0, available at www.biorxiv.org/content/10.1101/2019.12.20.884692vi.

In some embodiments, the first nucleobase is a modified or unmodified adenine, and the second nucleobase is a modified or unmodified adenine. In some embodiments, the modified adenine is N6-methyladenine (mA). In some embodiments, the modified adenine is one or more of N⁶-methyladenine (mA), N⁶-hydroxymethyladenine (hmA), or N⁶-formyladenine (fA).

### Captured Set; Target Regions

In some embodiments, nucleic acids captured or enriched using a method described herein comprise captured DNA, such as one or more captured sets of DNA. In some embodiments, the captured DNA comprise target regions that are differentially methylated in different immune cell types. In some embodiments, the immune cell types comprise rare or closely related immune cell types, such as activated and naive lymphocytes or myeloid cells at different stages of differentiation.

In some embodiments, a captured epigenetic target region set captured from a sample or first subsample includes hypermethylation variable target regions. In some embodiments, the hypermethylation variable target regions are differentially or exclusively hypermethylated in one cell type or in one immune cell type, or in one immune cell type within a cluster. In some embodiments, the hypermethylation variable target regions are hypermethylated to an extent that is distinguishably higher or exclusively present in one cell type or one immune cell type or one immune cell type within a cluster. Such hypermethylation variable target regions may be hypermethylated in other cell types but not to the extent observed in the one cell type. In some embodiments, the hypermethylation variable target regions show lower methylation in healthy cfDNA than in at least one other tissue type.

In some embodiments, a captured epigenetic target region set captured from a sample or second subsample includes hypomethylation variable target regions. In some embodiments, the hypomethylation variable target regions are exclusively hypomethylated in one cell type or in one immune cell type or in one immune cell type within a cluster. In some embodiments, the hypomethylation variable target regions are hypomethylated to an extent that is exclusively present in one cell type or one immune cell type or in one immune cell type within a cluster.

Such hypomethylation variable target regions may be hypomethylated in other cell types but not to the extent observed in the one cell type. In some embodiments, the hypomethylation variable target regions show higher methylation in healthy cfDNA than in at least one other tissue type. [0248] Without wishing to be bound by any particular theory, in an individual with cancer, proliferating or activated immune cells (and potentially also cancer cells) may shed more DNA into the bloodstream than immune cells in a healthy individual (and healthy cells of the same tissue type, respectively). As such, the distribution of cell type and/or tissue of origin of cfDNA may change upon carcinogenesis. For example, the distribution of immune cell type of origin may change in a subject having cancer, precancer, infection, transplant rejection, or other disease or disorder directly or indirectly affecting the immune system. The status of epigenetic target regions of certain immune cell types likewise may change in a subject having such a disease relative to a healthy subject or relative to the same subject prior to having the disease or disorder. Thus, variations in hypermethylation and/or hypomethylation can be an indicator of disease. For example, an increase in the level of hypermethylation variable target regions and/or hypomethylation variable target regions in a subsample following a partitioning step can be an indicator of the presence (or recurrence, depending on the history of the subject) of cancer.

Exemplary hypermethylation variable target regions and hypomethylation variable target regions useful for distinguishing between various cell types, including but not limited to immune cell types, have been identified by analyzing DNA obtained from various cell types via whole genome bisulfite sequencing, as described, e.g., in Stunnenberg, H. G. et. al., “The International Human Epigenome Consortium: A Blueprint for Scientific Collaboration and Discovery,” Cell 167, 1145 (2016) (doi.org/10.1186/s13059-020-02065-5). Whole-genome bisulfite sequencing data is available from the Blueprint consortium, available on the internet at dcc.blueprint-epigenome.eu.

In some embodiments, first and second captured target region sets comprise, respectively, DNA corresponding to a sequence-variable target region set and DNA corresponding to an epigenetic target region set, for example, as described in WO 2020/160414. The first and second captured sets may be combined to provide a combined captured set.

Where DNA (e.g., a sample or subsample) has been subjected to a procedure such as bisulfite conversion, treatment with a deaminase, or any of the other such procedures mentioned herein that alter the base-pairing specificity of certain bases, enrichment or capture may use oligonucleotides (e.g., primers or probes) specific for the altered or unaltered sequence, as desired.

In some embodiments in which a captured set comprising DNA corresponding to the sequence-variable target region set and the epigenetic target region set includes a combined captured set as discussed above, the DNA corresponding to the sequence-variable target region set may be present at a greater concentration than the DNA corresponding to the epigenetic target region set, e.g., a 1.1 to 1.2-fold greater concentration, a 1.2- to 1.4-fold greater concentration, a 1.4- to 1.6-fold greater concentration, a 1.6- to 1.8-fold greater concentration, a 1.8- to 2.0-fold greater concentration, a 2.0- to 2.2-fold greater concentration, a 2.2- to 2.4-fold greater concentration a 2.4- to 2.6-fold greater concentration, a 2.6- to 2.8-fold greater concentration, a 2.8- to 3.0-fold greater concentration, a 3.0- to 3.5-fold greater concentration, a 3.5- to 4.0, a 4.0- to 4.5-fold greater concentration, a 4.5- to 5.0-fold greater concentration, a 5.0- to 5.5-fold greater concentration, a 5.5- to 6.0-fold greater concentration, a 6.0- to 6.5-fold greater concentration, a 6.5- to 7.0-fold greater, a 7.0- to 7.5-fold greater concentration, a 7.5- to 8.0-fold greater concentration, an 8.0- to 8.5-fold greater concentration, an 8.5- to 9.0-fold greater concentration, a 9.0- to 9.5-fold greater concentration, 9.5- to 10.0-fold greater concentration, a 10- to 11-fold greater concentration, an 11- to 12-fold greater concentration a 12- to 13-fold greater concentration, a 13- to 14-fold greater concentration, a 14- to 15-fold greater concentration, a 15- to 16-fold greater concentration, a 16- to 17-fold greater concentration, a 17- to 18-fold greater concentration, an 18- to 19-fold greater concentration, a 19- to 20-fold greater concentration, a 20- to 30-fold greater concentration, a 30- to 40-fold greater concentration, a 40- to 50-fold greater concentration, a 50- to 60-fold greater concentration, a 60- to 70-fold greater concentration, a 70- to 80-fold greater concentration, a 80- to 90-fold greater concentration, or a 90- to 100-fold greater concentration. The degree of difference in concentrations accounts for normalization for the footprint sizes of the target regions, as discussed in the definition section

### Epigenetic Target Region Set

In some embodiments, an epigenetic target region set may comprise one or more types of target regions likely to differentiate DNA from different immune cell types and other non-immune cell types and/or to differentiate neoplastic (e.g., tumor or cancer) cells and from healthy cells, e.g., non-neoplastic circulating cells. Exemplary types of such regions are discussed in detail herein. The epigenetic target region set may also comprise one or more control regions, e.g., as described herein.

In some embodiments, the epigenetic target region set has a footprint of at least 100 kb, e.g., at least 200 kb, at least 300 kb, or at least 400 kb. In some embodiments, the epigenetic target region set has a footprint in the range of 100-1000 kb, e.g., 100-200 kb, 200-300 kb, 300-400 kb, 400-500 kb, 500-600 kb, 600-700 kb, 700-800 kb, 800-900 kb, and 900-1,000 kb.

### Hypermethylation Target Regions

In some embodiments, the epigenetic target region set includes one or more hypermethylation variable target regions. In some embodiments, hypermethylation variable target regions are exclusively hypermethylated in one immune cell type or hypermethylated to a greater extent in one immune cell type than in any other immune cell type or than in any other immune cell type within the same immune cell cluster. In some such embodiments, hypermethylation variable target regions indicate the levels of particular immune cell types from which the DNA originated, including rare immune cell types such as activated B cells (including memory B cells and plasma cells), activated T cells (including regulatory T cells (Tregs), CD4 effector memory T cells, CD4 central memory T cells, CD8 effector memory T cells, and CD8 central memory T cells), and natural killer (NK) cells. Methylation patterns of hypermethylation variable target regions that are useful for deconvoluting immune cell types may further change in certain disease states, such as cancer. Thus, in some embodiments, hypermethylation variable target regions that are useful for deconvoluting immune cell types are also useful for determining the likelihood that the subject from which the sample was obtained has cancer or precancer. In some such embodiments, hypermethylation variable target regions are useful for determining whether levels of particular immune cell types are abnormal and whether such abnormal levels are likely related to the presence of cancer or precancer, or if they are related to a different disease or condition other than cancer or precancer.

In some embodiments, certain hypermethylation variable target regions exhibit an increase in the level of observed methylation, e.g., are hypermethylated, in DNA produced by neoplastic cells, such as tumor or cancer cells. Detection of such hypermethylation variable target regions, e.g., in conjunction with detection of hypermethylation variable target regions indicative of immune cell types, may further increase the specificity and/or sensitivity of methods described herein. In some embodiments, such increases in observed methylation in hypermethylated variable target regions indicate an increased likelihood that a sample (e.g., of cfDNA) was obtained from a subject having cancer. For example, hypermethylation of promoters of tumor suppressor genes has been observed repeatedly. See, e.g., Kang et ah, Genome Biol. 18:53 (2017) and references cited therein. In another example, as discussed above, hypermethylation variable target regions can include regions that do not necessarily differ in methylation in cancerous tissue relative to DNA from healthy tissue of the same type, but do differ in methylation (e.g., have more methylation) relative to cfDNA that is typical in healthy subjects. Where, for example, the presence of a cancer results in increased cell death such as apoptosis of cells of the tissue type corresponding to the cancer, such a cancer can be detected at least in part using such hypermethylation variable target regions. In some embodiments, hypermethylation variable target regions useful for determining the likelihood that a subject has cancer are different than the hypermethylation variable target regions useful for determining the levels of particular immune cell types. In some embodiments, at least some of the hypermethylation variable target regions useful for determining the likelihood that a subject has cancer are the same as the hypermethylation variable target regions useful for determining the levels of particular immune cell types.

### Subjects

In some embodiments, the DNA (e.g., cfDNA) is obtained from a subject having a cancer or a precancer, an infection, transplant rejection, or other disease directly or indirectly affecting the immune system. In some embodiments, the DNA (e.g., cfDNA) is obtained from a subject suspected of having a cancer or a precancer, an infection, transplant rejection, or other disease directly or indirectly affecting the immune system. In some embodiments, the DNA (e.g., cfDNA) is obtained from a subject having a tumor. In some embodiments, the DNA (e.g., cfDNA) is obtained from a subject suspected of having a tumor. In some embodiments, the DNA (e.g., cfDNA) is obtained from a subject having neoplasia. In some embodiments, the DNA (e.g., cfDNA) is obtained from a subject suspected of having neoplasia. In some embodiments, the DNA (e.g., cfDNA) is obtained from a subject in remission from a tumor, cancer, or neoplasia (e.g., following chemotherapy, surgical resection, radiation, or a combination thereof). In any of the foregoing embodiments, the cancer, tumor, or neoplasia or suspected cancer, tumor, or neoplasia may be of the lung, colon, rectum, kidney, breast, prostate, or liver. In some embodiments, the cancer, tumor, or neoplasia or suspected cancer, tumor, or neoplasia is of the lung. In some embodiments, the cancer, tumor, or neoplasia or suspected cancer, tumor, or neoplasia is of the colon or rectum. In some embodiments, the cancer, tumor, or neoplasia or suspected cancer, tumor, or neoplasia is of the breast. In some embodiments, the cancer, tumor, or neoplasia or suspected cancer, tumor, or neoplasia is of the prostate. In any of the foregoing embodiments, the subject may be a human subject.

Pooling of DNA from Samples or Subsamples or Portions Thereof

In some embodiments, the methods herein comprise preparing one or more pools comprising tagged DNA from a plurality of partitioned subsamples. In some embodiments, a pool includes at least a portion of the DNA of a hypomethylated partition and at least a portion of the DNA of a hypermethylated partition. Target regions, e.g., including epigenetic target regions and/or sequence-variable target regions, may be captured from a pool. The steps of capturing a target region set from at least an aliquot or portion of a sample or subsample described elsewhere herein encompass capture steps performed on a pool comprising DNA from first and second subsamples. A step of amplifying DNA in a pool may be performed before capturing target regions from the pool. The capturing step may have any of the features described for capturing steps elsewhere herein.

In some embodiments, the methods comprise preparing a first pool comprising at least a portion of the DNA of a hypomethylated partition. In some embodiments, the methods comprise preparing a second pool comprising at least a portion of the DNA of a hypermethylated partition. In some embodiments, the methods comprise capturing at least a first set of target regions from the first pool, wherein the first set includes sequence-variable target regions. A step of amplifying DNA in the first pool may be performed before this capture step. In some embodiments, capturing the first set of target regions from the first pool includes contacting the DNA of the first pool with a first set of target-specific probes, wherein the first set of target-specific probes includes target-binding probes specific for the sequence-variable target regions. In some embodiments, the methods comprise capturing a second plurality of sets of target regions from the second pool, wherein the second plurality includes sequence-variable target regions and epigenetic target regions. A step of amplifying DNA in the second pool may be performed before this capture step. In some embodiments, capturing the second plurality of sets of target regions from the second pool includes contacting the DNA of the first pool with a second set of target-specific probes, wherein the second set of target-specific probes includes target-binding probes specific for the sequence-variable target regions and target-binding probes specific for the epigenetic target regions.

In some embodiments, sequence-variable target regions are captured from a second portion of a partitioned subsample. The second portion may include some, a majority, substantially all, or all of the DNA of the subsample that was not included in the pool. The regions captured from the pool and from the subsample may be combined and analyzed in parallel.

The epigenetic target regions may show differences in methylation levels and/or fragmentation patterns depending on whether they originated from a particular cell or tissue type or from a tumor or from healthy cells, as discussed elsewhere herein. The sequence-variable target regions may show differences in sequence depending on whether they originated from a tumor or from healthy cells. [0293] Analysis of epigenetic target regions from a hypomethylated partition may be less informative in some applications than analysis of sequence-variable target regions from hypermethylated and hypomethylated partitions and epigenetic target regions from a hypermethylated partition. As such, in methods where sequence-variable target regions and epigenetic target regions are being captured, the latter may be captured to a lesser extent than one or more of the sequence-variable target regions are captured from the hypermethylated and hypomethylated partitions and/or to a lesser extent that epigenetic target regions are captured from a hypermethylated partition. For example, sequence-variable target regions can be captured from a portion of a hypomethylated partition that is not pooled with a hypermethylated partition, and the pool can be prepared with some (e.g., a majority, substantially all, or all) of the DNA from a hypermethylated partition and none or some (e.g., a minority) of the DNA from a hypomethylated partition. Such approaches can reduce or eliminate sequencing of epigenetic target regions from hypomethylated partitions, thereby reducing the amount of sequencing data that suffices for further analysis.

In some embodiments, including a minority of the DNA of a hypomethylated partition in the pool facilitates quantification of one or more epigenetic features (e.g., methylation or other epigenetic feature(s) discussed in detail elsewhere herein), e.g., on a relative basis.

In some embodiments, the pool includes a minority of the DNA of a hypomethylated partition, e.g., less than about 50% of the DNA of a hypomethylated partition, such as less than or equal to about 45%, 40%, 35%, 30%, 25%, 20%, 15%, 10%, or 5% of the DNA of a hypomethylated partition. In some embodiments, the pool includes about 5%-25% of the DNA of a hypomethylated partition. In some embodiments, the pool includes about 10%-20% of the DNA of a hypomethylated partition. In some embodiments, the pool includes about 10% of the DNA of a hypomethylated partition. In some embodiments, the pool includes about 15% of the DNA of a hypomethylated partition. In some embodiments, the pool includes about 20% of the DNA of a hypomethylated partition.

In some embodiments, the pool includes a portion of a hypermethylated partition, which may be at least about 50% of the DNA of a hypermethylated partition. For example, the pool may comprise at least about 55%, 60%, 65%, 70%, 75%, 80%, 85%, 90%, or 95% of the DNA of a hypermethylated partition. In some embodiments, the pool includes 50-55%, 55-60%, 60-65%, 65-70%, 70-75%, 75-80%, 80-85%, 85-90%, 90-95%, or 95-100% of the DNA of a hypermethylated partition. In some embodiments, the second pool includes all or substantially all of the DNA of a hypermethylated partition.

In some embodiments, a first pool includes substantially all or all of the DNA of a hypomethylated partition (e.g., wherein a second pool does not comprise DNA of a hypomethylated partition. In some embodiments, the second pool does not comprise DNA of a hypomethylated partition (e.g., wherein the first pool includes substantially all or all of the DNA of a hypomethylated partition).

In some embodiments, a second pool includes a portion of a hypermethylated partition, which may be any of the values and ranges set forth above with respect to a hypomethylated partition. In some embodiments, the second pool includes all or substantially all of the DNA of a hypermethylated partition.

In an exemplary embodiment, after partitioning, the partitions separately undergo end repair and ligation to adapters comprising molecular barcodes and are then amplified separately. After the amplification, amplified molecules are enriched (still keeping the partitions separate). Post-enrichment, the enriched DNA are pooled according to any of the embodiments described herein, and then amplified again. After amplification, the molecules are sequenced.

In various embodiments, the methods further comprise sequencing the captured DNA, e.g., to different degrees of sequencing depth for the epigenetic and sequence-variable target region sets, consistent with the discussion above.

### Sequencing

In general, sample nucleic acids, including nucleic acids flanked by adapters, with or without prior amplification can be subject to sequencing. Sequencing methods include, for example, Sanger sequencing, high-throughput sequencing, pyrosequencing, sequencing-by synthesis, single-molecule sequencing, nanopore sequencing, semiconductor sequencing, sequencing-by-ligation, sequencing-by-hybridization, Digital Gene Expression (Helicos), Next generation sequencing (NGS), Single Molecule Sequencing by Synthesis (SMSS) (Helicos), massively-parallel sequencing, Clonal Single Molecule Array (Solexa), shotgun sequencing, Ion Torrent, Oxford Nanopore, Roche Genia, Maxim-Gilbert sequencing, primer walking, and sequencing using PacBio, SOLiD, Ion Torrent, or Nanopore platforms.

In some embodiments, sequencing includes detecting and/or distinguishing unmodified and modified nucleobases. For example, PacBio sequencing (e.g., single-molecule real-time (SMRT) sequencing) offers the ability to directly detect of, e.g., 5-methylcytosine and 5-hydroxymethylcytosine as well as unmodified cytosine. See, e.g., Schatz., Nature Methods. 14(4): 347-348 (2017); and U.S. Pat. No. 9,150,918. Also, Oxford nanopore sequencing systems (e.g., MinION sequencer) that can directly detect methylation of DNA (for example: 5-methylcytosine and 5-hydroxymethylcytosine) can be used here. Sequencing reactions can be performed in a variety of sample processing units, which may multiple lanes, multiple channels, multiple wells, or other mean of processing multiple sample sets substantially simultaneously. Sample processing unit can also include multiple sample chambers to enable processing of multiple runs simultaneously. Similarly, Ion Torrent sequencing may also be used to directly detect methylation. Thus, in some embodiments, methylation status can be determined during sequencing, e.g., without or independently of a partitioning step or a conversion procedure such as bisulfite treatment.

The sequencing reactions can be performed on one or more forms of nucleic acids, such as those known to contain markers of cancer or of other disease. The sequencing reactions can also be performed on any nucleic acid fragments present in the sample. In some embodiments, sequence coverage of the genome may be less than 5%, 10%, 15%, 20%, 25%, 30%, 40%, 50%, 60%, 70%, 80%, 90%, 95%, 99%, 99.9% or 100%. In some embodiments, the sequence reactions may provide for sequence coverage of at least 5%, 10%, 15%, 20%, 25%, 30%, 40%, 50%, 60%, 70%, or 80% of the genome. Sequence coverage can performed on at least 5, 10, 20, 70, 100, 200 or 500 different genes, or at most 5000, 2500, 1000, 500 or 100 different genes. [0304] Simultaneous sequencing reactions may be performed using multiplex sequencing. In some cases, cell-free nucleic acids may be sequenced with at least 1000, 2000, 3000, 4000, 5000, 6000, 7000, 8000, 9000, 10000, 50000, 100,000 sequencing reactions. In other cases cell-free nucleic acids may be sequenced with less than 1000, 2000, 3000, 4000, 5000, 6000, 7000, 8000, 9000, 10000, 50000, 100,000 sequencing reactions. Sequencing reactions may be performed sequentially or simultaneously. Subsequent data analysis may be performed on all or part of the sequencing reactions. In some cases, data analysis may be performed on at least 1000, 2000, 3000, 4000, 5000, 6000, 7000, 8000, 9000, 10000, 50000, 100,000 sequencing reactions. In other cases, data analysis may be performed on less than 1000, 2000, 3000, 4000, 5000, 6000, 7000, 8000, 9000, 10000, 50000, 100,000 sequencing reactions. An exemplary read depth is 1000-50000 reads per locus (base). 1.

### Differential Depth of Sequencing

In some embodiments, nucleic acids corresponding to a sequence-variable target region set are sequenced to a greater depth of sequencing than nucleic acids corresponding to an epigenetic target region set. For example, the depth of sequencing for nucleic acids corresponding to sequence variant target region sets may be at least 1.25-, 1.5-, 1.75-, 2-, 2.25-, 2.5-, 2.75-, 3-, 3.5-, 4-, 4.5-, 5-, 6-, 7-, 8-, 9-, 10-, 11-, 12-, 13-, 14-, or 15-fold greater, or 1.25- to 1.5-, 1.5- to 1.75-, 1.75- to 2-, 2- to 2.25-, 2.25- to 2.5-, 2.5- to 2.75-, 2.75- to 3-, 3- to 3.5-, 3.5- to 4-, 4- to 4.5-, 4.5- to 5-, 5- to 5.5-, 5.5- to 6-, 6- to 7-, 7- to 8-, 8- to 9-, 9- to 10-, 10- to 11-, 11- to 12-, 13- to 14-, 14- to 15-fold, or 15- to 100-fold greater, than the depth of sequencing for nucleic acids corresponding to an epigenetic target region set. In some embodiments, said depth of sequencing is at least 2-fold greater. In some embodiments, said depth of sequencing is at least 5-fold greater. In some embodiments, said depth of sequencing is at least 10-fold greater. In some embodiments, said depth of sequencing is 4- to 10-fold greater. In some embodiments, said depth of sequencing is 4- to 100-fold greater.

In some embodiments, DNA corresponding to a sequence-variable target region set, and/or to an epigenetic target region set are sequenced concurrently, e.g., in the same sequencing cell (such as the flow cell of an Illumina sequencer) and/or in the same composition, which may be a combined or pooled composition resulting from recombining separately captured sets or a composition obtained by, e.g., capturing the cfDNA corresponding to the sequence-variable target region set, and/or the captured cfDNA corresponding to an epigenetic target region set in the same vessel.

### Samples

A sample can be any biological sample isolated from a subject. A sample can be a bodily sample. Samples can include body tissues, such as known or suspected solid tumors, whole blood, platelets, serum, plasma, stool, red blood cells, white blood cells or leucocytes, endothelial cells, tissue biopsies, cerebrospinal fluid synovial fluid, lymphatic fluid, ascites fluid, interstitial or extracellular fluid, the fluid in spaces between cells, including gingival crevicular fluid, bone marrow, pleural effusions, cerebrospinal fluid, saliva, mucous, sputum, semen, sweat, urine. Samples are preferably body fluids, particularly blood and fractions thereof, and urine. A sample can be in the form originally isolated from a subject or can have been subjected to further processing to remove or add components, such as cells, or enrich for one component relative to another. Thus, a preferred body fluid for analysis is plasma or serum containing cell-free nucleic acids.

In some embodiments, a population of nucleic acids is obtained from a serum, plasma or blood sample from a subject suspected of having neoplasia, a tumor, precancer, or cancer or previously diagnosed with neoplasia, a tumor, precancer, or cancer. The population includes nucleic acids having varying levels of sequence variation, epigenetic variation, and/or post replication or transcriptional modifications. Post-replication modifications include modifications of cytosine, particularly at the 5-position of the nucleobase, e.g., 5-methylcytosine, 5-hydroxymethylcytosine, 5-formylcytosine and 5-carboxylcytosine.

A sample can be isolated or obtained from a subject and transported to a site of sample analysis. The sample may be preserved and shipped at a desirable temperature, e.g., room temperature, 4° C., −20° C., and/or −80° C. A sample can be isolated or obtained from a subject at the site of the sample analysis. The subject can be a human, a mammal, an animal, a companion animal, a service animal, or a pet. The subject may have a cancer, precancer, infection, transplant rejection, or other disease or disorder related to changes in the immune system. The subject may not have cancer or a detectable cancer symptom. The subject may have been treated with one or more cancer therapy, e.g., any one or more of chemotherapies, antibodies, vaccines or biologies. The subject may be in remission. The subject may or may not be diagnosed of being susceptible to cancer or any cancer-associated genetic mutations/disorders.

In some embodiments, the sample includes plasma. The volume of plasma obtained can depend on the desired read depth for sequenced regions. Exemplary volumes are 0.4-40 ml, 5-20 ml, 10-20 ml. For examples, the volume can be 0.5 mL, 1 mL, 5 mL 10 mL, 20 mL, 30 mL, or 40 mL. A volume of sampled plasma may be 5 to 20 mL.

A sample can comprise various amount of nucleic acid that contains genome equivalents. For example, a sample of about 30 ng DNA can contain about 10,000 (10⁴) haploid human genome equivalents and, in the case of cfDNA, about 200 billion (2×10ⁿ) individual polynucleotide molecules. Similarly, a sample of about 100 ng of DNA can contain about 30,000 haploid human genome equivalents and, in the case of cfDNA, about 600 billion individual molecules.

A sample can comprise nucleic acids from different sources, e.g., from cells and cell-free of the same subject, from cells and cell-free of different subjects. A sample can comprise nucleic acids carrying mutations. For example, a sample can comprise DNA carrying germline mutations and/or somatic mutations. Germline mutations refer to mutations existing in germline DNA of a subject. Somatic mutations refer to mutations originating in somatic cells of a subject, e.g., precancer cells or cancer cells. A sample can comprise DNA carrying cancer-associated mutations (e.g., cancer-associated somatic mutations). A sample can comprise an epigenetic variant (i.e. a chemical or protein modification), wherein the epigenetic variant associated with the presence of a genetic variant such as a cancer-associated mutation. In some embodiments, the sample includes an epigenetic variant associated with the presence of a genetic variant, wherein the sample does not comprise the genetic variant.

Exemplary amounts of cell-free nucleic acids in a sample before amplification range from about 1 fg to about 1 pg, e.g., 1 μg to 200 ng, 1 ng to 100 ng, 10 ng to 1000 ng. For example, the amount can be up to about 600 ng, up to about 500 ng, up to about 400 ng, up to about 300 ng, up to about 200 ng, up to about 100 ng, up to about 50 ng, or up to about 20 ng of cell-free nucleic acid molecules. The amount can be at least 1 fg, at least 10 fg, at least 100 fg, at least 1 pg, at least 10 pg, at least 100 pg, at least 1 ng, at least 10 ng, at least 100 ng, at least 150 ng, or at least 200 ng of cell-free nucleic acid molecules. The amount can be up to 1 femtogram (fg), 10 fg, 100 fg, 1 picogram (pg), 10 μg, 100 pg, 1 ng, 10 ng, 100 ng, 150 ng, or 200 ng of cell-free nucleic acid molecules. The method can comprise obtaining 1 femtogram (fg) to 200 ng-[0326] Cell-free nucleic acids are nucleic acids not contained within or otherwise bound to a cell or in other words nucleic acids remaining in a sample after removing intact cells. Cell-free nucleic acids include DNA, RNA, and hybrids thereof, including genomic DNA, mitochondrial DNA, siRNA, miRNA, circulating RNA (cRNA), tRNA, rRNA, small nucleolar RNA (snoRNA), Piwi-interacting RNA (piRNA), long non-coding RNA (long ncRNA), or fragments of any of these. Cell-free nucleic acids can be double-stranded, single-stranded, or a hybrid thereof. A cell-free nucleic acid can be released into bodily fluid through secretion or cell death processes, e.g., cellular necrosis and apoptosis. Some cell-free nucleic acids are released into bodily fluid from cancer cells e.g., circulating tumor DNA, (ctDNA). Others are released from healthy cells. In some embodiments, cfDNA is cell-free fetal DNA (cffDNA) In some embodiments, cell free nucleic acids are produced by tumor cells. In some embodiments, cell free nucleic acids are produced by a mixture of tumor cells and non-tumor cells.

Cell-free nucleic acids have an exemplary size distribution of about 100-500 nucleotides, with molecules of 110 to about 230 nucleotides representing about 90% of molecules, with a mode of about 168 nucleotides and a second minor peak in a range between 240 to 440 nucleotides.

Cell-free nucleic acids can be isolated from bodily fluids through a fractionation step in which cell-free nucleic acids, as found in solution, are separated from intact cells and other non soluble components of the bodily fluid. Partitioning may include techniques such as centrifugation or filtration. Alternatively, cells in bodily fluids can be lysed and cell-free and cellular nucleic acids processed together. Generally, after addition of buffers and wash steps, nucleic acids can be precipitated with an alcohol. Further clean up steps may be used such as silica based columns to remove contaminants or salts. Non-specific bulk carrier nucleic acids, such as C 1 DNA, DNA or protein for bisulfite sequencing, hybridization, and/or ligation, may be added throughout the reaction to optimize certain aspects of the procedure such as yield.

After such processing, samples can include various forms of nucleic acid including double stranded DNA, single stranded DNA, and single stranded RNA. In some embodiments, single stranded DNA and RNA can be converted to double stranded forms so they are included in subsequent processing and analysis steps.

DNA molecules can be linked to adapters at either one end or both ends. Typically, double-stranded molecules are blunt ended by treatment with a polymerase with a 5′-3′ polymerase and a 3′-5′ exonuclease (or proof-reading function), in the presence of all four standard nucleotides. Klenow large fragment and T4 polymerase are examples of suitable polymerase. The blunt ended DNA molecules can be ligated with at least partially double stranded adapter (e.g., a Y shaped or bell-shaped adapter). Alternatively, complementary nucleotides can be added to blunt ends of sample nucleic acids and adapters to facilitate ligation. Contemplated herein are both blunt end ligation and sticky end ligation. In blunt end ligation, both the nucleic acid molecules and the adapter tags have blunt ends. In sticky-end ligation, typically, the nucleic acid molecules bear an “A” overhang and the adapters bear a “T” overhang.

### Amplification

Sample nucleic acids flanked by adapters can be amplified by PCR and other amplification methods. Amplification is typically primed by primers that anneal or bind to primer binding sites in adapters flanking a DNA molecule to be amplified. Amplification methods can involve cycles of denaturation, annealing and extension, resulting from thermocycling or can be isothermal as in transcription-mediated amplification. Other amplification methods include the ligase chain reaction, strand displacement amplification, nucleic acid sequence based amplification, and self-sustained sequence based replication.

In some embodiments, the present methods perform dsDNA ligations with T-tailed and C-tailed adapters, which result in amplification of at least 50, 60, 70 or 80% of double stranded nucleic acids before linking to adapters. Preferably the present methods increase the amount or number of amplified molecules relative to control methods performed with T-tailed adapters alone by at least 10, 15 or 20%.

### Tags

Tags comprising barcodes can be incorporated into or otherwise joined to adapters. Tags can be incorporated by ligation, overlap extension PCR among other methods.

### Molecular Tagging Strategies

Molecular tagging refers to a tagging practice that allows one to differentiate among DNA molecules from which sequence reads originated. Tagging strategies can be divided into unique tagging and non-unique tagging strategies. In unique tagging, all or substantially all of the molecules in a sample bear a different tag, so that reads can be assigned to original molecules based on tag information alone. Tags used in such methods are sometimes referred to as “unique tags”. In non-unique tagging, different molecules in the same sample can bear the same tag, so that other information in addition to tag information is used to assign a sequence read to an original molecule. Such information may include start and stop coordinate, coordinate to which the molecule maps, start or stop coordinate alone, etc. Tags used in such methods are sometimes referred to as “non-unique tags”. Accordingly, it is not necessary to uniquely tag every molecule in a sample. It suffices to uniquely tag molecules falling within an identifiable class within a sample. Thus, molecules in different identifiable families can bear the same tag without loss of information about the identity of the tagged molecule.

In certain embodiments of non-unique tagging, the number of different tags used can be sufficient that there is a very high likelihood (e.g., at least 99%, at least 99.9%, at least 99.99% or at least 99.999% that all DNA molecules of a particular group bear a different tag. It is to be noted that when barcodes are used as tags, and when barcodes are attached, e.g., randomly, to both ends of a molecule, the combination of barcodes, together, can constitute a tag. This number, in term, is a function of the number of molecules falling into the calls. For example, the class may be all molecules mapping to the same start-stop position on a reference genome. The class may be all molecules mapping across a particular genetic locus, e.g., a particular base or a particular region (e.g., up to 100 bases or a gene or an exon of a gene). In certain embodiments, the number of different tags used to uniquely identify a number of molecules, z, in a class can be between any of 2*z, 3*z, 4*z, 5*z, 6*z, 7*z, 8*z, 9*z, 10*z, 11*z, 12*z, 13*z, 14*z, 15*z, 16*z, 17*z, 18*z, 19*z, 20*z or 100*z (e.g., lower limit) and any of 100,000*z, 10,000*z, 1000*z or 100*z (e.g., upper limit).

For example, in a sample of about 5 ng to 30 ng of cell free DNA, one expects around 3000 molecules to map to a particular nucleotide coordinate, and between about 3 and 10 molecules having any start coordinate to share the same stop coordinate. Accordingly, about 50 to about 50,000 different tags (e.g., between about 6 and 220 barcode combinations) can suffice to uniquely tag all such molecules. To uniquely tag all 3000 molecules mapping across a nucleotide coordinate, about 1 million to about 20 million different tags would be required. [0336] Generally, assignment of unique or non-unique tags barcodes in reactions follows methods and systems described by US patent applications 20010053519, 20030152490, 20110160078, and U.S. Pat. Nos. 6,582,908 and 7,537,898 and 9,598,731. Tags can be linked to sample nucleic acids randomly or non-randomly. [0337] The unique tags may be loaded so that more than about 1, 2, 3, 4, 5, 6, 7, 8, 9, 10, 20, 50, 100, 500, 1000, 5000, 10000, 50,000, 100,000, 500,000, 1,000,000, 10,000,000, 50,000,000 or 1,000,000,000 unique tags are loaded per genome sample. In some cases, the unique tags may be loaded so that less than about 2, 3, 4, 5, 6, 7, 8, 9, 10, 20, 50, 100, 500, 1000, 5000, 10000, 50,000, 100,000, 500,000, 1,000,000, 10,000,000, 50,000,000 or 1,000,000,000 unique tags are loaded per genome sample. In some cases, the average number of unique tags loaded per sample genome is less than, or greater than, about 1, 2, 3, 4, 5, 6, 7, 8, 9, 10, 20, 50, 100, 500, 1000, 5000, 10000, 50,000, 100,000, 500,000, 1,000,000, 10,000,000, 50,000,000 or 1,000,000,000 unique tags per genome sample.

A preferred format uses 20-50 different tags (e.g., barcodes) ligated to both ends of target nucleic acids. For example, 35 different tags (e.g., barcodes) ligated to both ends of target molecules creating 35×35 permutations, which equals 1225 for 35 tags. Such numbers of tags are sufficient so that different molecules having the same start and stop points have a high probability (e.g., at least 94%, 99.5%, 99.99%, 99.999%) of receiving different combinations of tags. Other barcode combinations include any number between 10 and 500, e.g., about 15×15, about 35×35, about 75×75, about 100×100, about 250×250, about 500×500.

In some cases, unique tags may be predetermined or random or semi-random sequence oligonucleotides. In other cases, a plurality of barcodes may be used such that barcodes are not necessarily unique to one another in the plurality. In this example, barcodes may be ligated to individual molecules such that the combination of the barcode and the sequence it may be ligated to creates a unique sequence that may be individually tracked. As described herein, detection of non-unique barcodes in combination with sequence data of beginning (start) and end (stop) portions of sequence reads may allow assignment of a unique identity to a particular molecule. The length or number of base pairs, of an individual sequence read may also be used to assign a unique identity to such a molecule. As described herein, fragments from a single strand of nucleic acid having been assigned a unique identity, may thereby permit subsequent identification of fragments from the parent strand.

### Capture Moieties

As discussed above, nucleic acids in a sample can be subject to a capture step, in which molecules having target regions are captured for subsequent analysis. Target capture can involve use of probes (e.g., oligonucleotides) labeled with a capture moiety, such as biotin, and a second moiety or binding partner that binds to the capture moiety, such as streptavidin. In some embodiments, a capture moiety and binding partner can have higher and lower capture yields for different sets of target regions, such as those of the sequence-variable target region set and the epigenetic target region set, respectively, as discussed elsewhere herein. Methods comprising capture moieties are further described in, for example, U.S. Pat. No. 9,850,523, issuing Dec. 26, 2017, which is incorporated herein by reference.

Capture moieties include, without limitation, biotin, avidin, streptavidin, a nucleic acid comprising a particular nucleotide sequence, a hapten recognized by an antibody, and magnetically attractable particles. The extraction moiety can be a member of a binding pair, such as biotin/streptavidin or hapten/antibody. In some embodiments, a capture moiety that is attached to an analyte is captured by its binding pair which is attached to an isolatable moiety, such as a magnetically attractable particle or a large particle that can be sedimented through centrifugation. The capture moiety can be any type of molecule that allows affinity separation of nucleic acids bearing the capture moiety from nucleic acids lacking the capture moiety. Exemplary capture moieties are biotin which allows affinity separation by binding to streptavidin linked or linkable to a solid phase or an oligonucleotide, which allows affinity separation through binding to a complementary oligonucleotide linked or linkable to a solid phase.

### Collections of Target-Specific Probes

In some embodiments, a collection of target-specific probes is used in a method comprising an epigenetic target region set and/or a sequence-variable target region set, as described herein. In some embodiments, the collection of target-specific probes includes target binding probes specific for a sequence-variable target region set and target-binding probes specific for an epigenetic target region set. In some embodiments, the capture yield of the target binding probes specific for the sequence-variable target region set is higher (e.g., at least 2-fold higher) than the capture yield of the target-binding probes specific for the epigenetic target region set. In some embodiments, the collection of target-specific probes is configured to have a capture yield specific for the sequence-variable target region set higher (e.g., at least 2-fold higher) than its capture yield specific for the epigenetic target region set.

In some embodiments, the capture yield of the target-binding probes specific for the sequence-variable target region set is at least 1.25-, 1.5-, 1.75-, 2-, 2.25-, 2.5-, 2.75-, 3-, 3.5-, 4-, 4.5-, 5-, 6-, 7-, 8-, 9-, 10-, 11-, 12-, 13-, 14-, or 15-fold higher than the capture yield of the target-binding probes specific for the epigenetic target region set. In some embodiments, the capture yield of the target-binding probes specific for the sequence-variable target region set is 1.25- to 1.5-, 1.5- to 1.75-, 1.75- to 2-, 2- to 2.25-, 2.25- to 2.5-, 2.5- to 2.75-, 2.75- to 3-, 3- to 3.5-, 3.5- to 4-, 4- to 4.5-, 4.5- to 5-, 5- to 5.5-, 5.5- to 6-, 6- to 7-, 7- to 8-, 8- to 9-, 9- to 10-, 10- to 11-, 11- to 12-, 13- to 14-, or 14- to 15-fold higher than the capture yield of the target-binding probes specific for the epigenetic target region set.

In some embodiments, the collection of target-specific probes is configured to have a capture yield specific for the sequence-variable target region set at least 1.25-, 1.5-, 1.75-, 2-, 2.25-, 2.5-, 2.75-, 3-, 3.5-, 4-, 4.5-, 5-, 6-, 7-, 8-, 9-, 10-, 11-, 12-, 13-, 14-, or 15-fold higher than its capture yield for the epigenetic target region set. In some embodiments, the collection of target-specific probes is configured to have a capture yield specific for the sequence-variable target region set is 1.25- to 1.5-, 1.5- to 1.75-, 1.75- to 2-, 2- to 2.25-, 2.25- to 2.5-, 2.5- to 2.75-, 2.75- to 3-, 3- to 3.5-, 3.5- to 4-, 4- to 4.5-, 4.5- to 5-, 5- to 5.5-, 5.5- to 6-, 6- to 7-, 7- to 8-, 8- to 9-, 9- to 10-, 10- to 11-, 11- to 12-, 13- to 14-, or 14- to 15-fold higher than its capture yield specific for the epigenetic target region set.

The collection of probes can be configured to provide higher capture yields for the sequence-variable target region set in various ways, including concentration, different lengths and/or chemistries (e.g., that affect affinity), and combinations thereof. Affinity can be modulated by adjusting probe length and/or including nucleotide modifications as discussed below.

In some embodiments, the target-specific probes specific for the sequence-variable target region set are present at a higher concentration than the target-specific probes specific for the epigenetic target region set. In some embodiments, concentration of the target-binding probes specific for the sequence-variable target region set is at least 1.25-, 1.5-, 1.75-, 2-, 2.25-, 2.5-, 2.75-, 3-, 3.5-, 4-, 4.5-, 5-, 6-, 7-, 8-, 9-, 10-, 11-, 12-, 13-, 14-, or 15-fold higher than the concentration of the target-binding probes specific for the epigenetic target region set. In some embodiments, the concentration of the target-binding probes specific for the sequence-variable target region set is 1.25- to 1.5-, 1.5- to 1.75-, 1.75- to 2-, 2- to 2.25-, 2.25- to 2.5-, 2.5- to 2.75-, 2.75- to 3-, 3- to 3.5-, 3.5- to 4-, 4- to 4.5-, 4.5- to 5-, 5- to 5.5-, 5.5- to 6-, 6- to 7-, 7- to 8-, 8- to 9-, 9- to 10-, 10- to 11-, 11- to 12-, 13- to 14-, or 14- to 15-fold higher than the concentration of the target-binding probes specific for the epigenetic target region set. In such embodiments, concentration may refer to the average mass per volume concentration of individual probes in each set.

In some embodiments, the target-specific probes specific for the sequence-variable target region set have a higher affinity for their targets than the target-specific probes specific for the epigenetic target region set. Affinity can be modulated in any way known to those skilled in the art, including by using different probe chemistries. For example, certain nucleotide modifications, such as cytosine 5-methylation (in certain sequence contexts), modifications that provide a heteroatom at the T sugar position, and LNA nucleotides, can increase stability of double-stranded nucleic acids, indicating that oligonucleotides with such modifications have relatively higher affinity for their complementary sequences. See, e.g., Severin et ah, Nucleic Acids Res. 39: 8740-8751 (2011); Freier et ah, Nucleic Acids Res. 25: 4429-4443 (1997); U.S. Pat. No. 9,738,894. Also, longer sequence lengths will generally provide increased affinity. Other nucleotide modifications, such as the substitution of the nucleobase hypoxanthine for guanine, reduce affinity by reducing the amount of hydrogen bonding between the oligonucleotide and its complementary sequence. In some embodiments, the target-specific probes specific for the sequence-variable target region set have modifications that increase their affinity for their targets. In some embodiments, alternatively or additionally, the target-specific probes specific for the epigenetic target region set have modifications that decrease their affinity for their targets. In some embodiments, the target-specific probes specific for the sequence-variable target region set have longer average lengths and/or higher average melting temperatures than the target-specific probes specific for the epigenetic target region set. These embodiments may be combined with each other and/or with differences in concentration as discussed above to achieve a desired fold difference in capture yield, such as any fold difference or range thereof described above.

In some embodiments, the target-specific probes comprise a capture moiety. The capture moiety may be any of the capture moieties described herein, e.g., biotin. In some embodiments, the target-specific probes are linked to a solid support, e.g., covalently or non-covalently such as through the interaction of a binding pair of capture moieties. In some embodiments, the solid support is a bead, such as a magnetic bead.

In some embodiments, the target-specific probes specific for the sequence-variable target region set and/or the target-specific probes specific for the epigenetic target region set comprise a capture moiety as discussed above, e.g., probes comprising capture moieties and sequences selected to tile across a panel of regions, such as genes.

In some embodiments, the target-specific probes are provided in a single composition.

The single composition may be a solution (liquid or frozen). Alternatively, it may be a lyophilizate.

Alternatively, the target-specific probes may be provided as a plurality of compositions, e.g., comprising a first composition comprising probes specific for the epigenetic target region set and a second composition comprising probes specific for the sequence-variable target region set. These probes may be mixed in appropriate proportions to provide a combined probe composition with any of the foregoing fold differences in concentration and/or capture yield. Alternatively, they may be used in separate capture procedures (e.g., with aliquots of a sample or sequentially with the same sample) to provide first and second compositions comprising captured epigenetic target regions and sequence-variable target regions, respectively.

### Probes Specific for Epigenetic Target Regions

The probes for the epigenetic target region set may comprise probes specific for one or more types of target regions likely to differentiate DNA originating from different types of immune cells, including rare immune cell types, and/or to differentiate DNA from precancerous or neoplastic (e.g., tumor or cancer) cells from healthy cells, e.g., non-neoplastic circulating cells. Exemplary types of such regions are discussed in detail herein. The probes for the epigenetic target region set may also comprise probes for one or more control regions, e.g., as described herein.

In some embodiments, the probes for the epigenetic target region probe set have a footprint of at least 100 kb, e.g., at least 200 kb, at least 300 kb, or at least 400 kb. In some embodiments, the probes for the epigenetic target region set have a footprint in the range of 100-1000 kb, e.g., 100-200 kb, 200-300 kb, 300-400 kb, 400-500 kb, 500-600 kb, 600-700 kb, 700-800 kb, 800-900 kb, and 900-1,000 kb. In some embodiments, the probes for the epigenetic target region probe set have a footprint of at least 5 kb, e.g., at least 10, 20, or 50 kb. a. Hypermethylation variable target regions.

In some embodiments, the probes for the epigenetic target region set comprise probes specific for one or more hypermethylation variable target regions. The hypermethylation variable target regions may be any of those set forth above. For example, in some embodiments, the probes specific for hypermethylation variable target regions comprise probes specific for a plurality of loci that are differentially methylated in different immune cell types. In some embodiments, each immune cell type specific hypermethylation variable target region includes at least one CpG site that is methylated with a frequency greater than or equal to 0.3, 0.4, 0.5, or 0.6 in one immune cell type and with a frequency less than or equal to 0.1, 0.2, or 0.3 in all other immune cell types. In some embodiments, each immune cell type specific hypermethylation variable target region includes at least two CpG sites within 100 base pairs of each other that are each methylated with a frequency greater than or equal to 0.3, 0.4, 0.5, or 0.6 in one immune cell type and with a frequency less than or equal to 0.1, 0.2, or 0.3 in all other immune cell types. In some such embodiments, each immune cell type specific hypermethylation variable target region includes a total of at least 2, 3, 4, 5, 6, 7, 8, 9, or 10 CpG sites within 150 base pairs or within 200 base pairs, wherein fewer than three of the at least 2, 3, 4, 5, 6, 7, 8, 9, or 10 CpG sites are methylated with a frequency greater than 0.1, 0.2, or 0.3 in any normal tissue type. In some embodiments, each immune cell type specific epigenetic target region set includes at least 3, at least 5, at least 10, at least 20, or at least 30 hypermethylation variable target regions that are uniquely hypermethylated in each one of the immune cell types that are identified in the method.

In some embodiments, the probes specific for hypermethylation variable target regions comprise probes specific for a plurality of loci listed in Table 1, e.g., at least 10%, 20%, 30%, 40%, 50%, 60%, 70%, 80%, 90%, or 100% of the loci listed in Table 1. In some embodiments, the probes specific for hypermethylation variable target regions comprise probes specific for a plurality of loci listed in Table 2, e.g., at least 10%, 20%, 30%, 40%, 50%, 60%, 70%, 80%, 90%, or 100% of the loci listed in Table 2. In some embodiments, the probes specific for hypermethylation variable target regions comprise probes specific for a plurality of loci listed in Table 1 or Table 2, e.g., at least 10%, 20%, 30%, 40%, 50%, 60%, 70%, 80%, 90%, or 100% of the loci listed in Table 1 or Table 2.

In some embodiments, for each locus included as a target region, there may be one or more probes with a hybridization site that binds between the transcription start site and the stop codon (the last stop codon for genes that are alternatively spliced) of the gene. In some embodiments, the one or more probes bind within 300 bp of the listed position, e.g., within 200 or 100 bp. In some embodiments, a probe has a hybridization site overlapping the position listed above. In some embodiments, the probes specific for the hypermethylation target regions include probes specific for one, two, three, four, or five subsets of hypermethylation target regions that collectively show hypermethylation in one, two, three, four, or five of breast, colon, kidney, liver, and lung cancers. b. Hypomethylation variable target regions.

In some embodiments, the probes for the epigenetic target region set comprise probes specific for one or more hypomethylation variable target regions. The hypomethylation variable target regions may be any of those set forth above. For example, in some embodiments, the probes specific for hypomethylation variable target regions comprise probes specific for a plurality of loci that are differentially methylated in different immune cell types. In some embodiments, each immune cell type specific hypomethylation variable target region includes at least one CpG site that is methylated with a frequency less than or equal to 0.1, 0.2, or 0.3 in one immune cell type and with a frequency greater than or equal to 0.3, 0.4, 0.5, or 0.6 in all other immune cell types. In some embodiments, each immune cell type specific hypomethylation variable target region includes at least two CpG sites within 100 base pairs of each other that are each methylated with a frequency less than or equal to 0.1, 0.2, or 0.3 in one immune cell type and with a frequency greater than or equal to 0.3, 0.4, 0.5, or 0.6 in all other immune cell types. In some such embodiments, each immune cell type specific hypomethylation variable target region includes a total of at least 2, 3, 4, 5, 6, 7, 8, 9, or 10 CpG sites within 150 base pairs or within 200 base pairs, wherein fewer than three of the at least 2, 3, 4, 5, 6, 7, 8, 9, or 10 CpG sites are methylated with a frequency less than 0.1, 0.2, or 0.3 in any normal tissue type. In some embodiments, each immune cell type specific epigenetic target region set includes at least 3, at least 5, at least 10, at least 20, or at least 30 hypomethylation variable target regions that are uniquely hypomethylated in each one of the immune cell types that are identified in the method.

In some embodiments, the probes specific for one or more hypomethylation variable target regions may include probes for regions such as repeated elements, e.g., LINE1 elements, Alu elements, centromeric tandem repeats, pericentromeric tandem repeats, and satellite DNA, and intergenic regions that are ordinarily methylated in healthy cells may show reduced methylation in tumor cells.

In some embodiments, probes specific for hypomethylation variable target regions include probes specific for repeated elements and/or intergenic regions. In some embodiments, probes specific for repeated elements include probes specific for one, two, three, four, or five of LINE1 elements, Alu elements, centromeric tandem repeats, pericentromeric tandem repeats, and/or satellite DNA.

Exemplary probes specific for genomic regions that show cancer-associated hypomethylation include probes specific for nucleotides 8403565-8953708 and/or 151104701-151106035 of human chromosome 1. In some embodiments, the probes specific for hypomethylation variable target regions include probes specific for regions overlapping or comprising nucleotides 8403565-8953708 and/or 151104701-151106035 of human chromosome 1 c.

### Focal Amplifications

As noted above, although focal amplifications are somatic mutations, they can be detected by sequencing based on read frequency in a manner analogous to approaches for detecting certain epigenetic changes such as changes in methylation. As such, regions that may show focal amplifications in cancer can be included in the epigenetic target region set, as discussed above. In some embodiments, the probes specific for the epigenetic target region set include probes specific for focal amplifications. In some embodiments, the probes specific for focal amplifications include probes specific for one or more of AR, BRAF, CCND1, CCND2, CCNE1, CDK4, CDK6, EGFR, ERBB2, FGFR1, FGFR2, KIT, KRAS, MET, MYC, PDGFRA, PIK3CA, and RAFI. For example, in some embodiments, the probes specific for focal amplifications include probes specific for one or more of at least 2, 3, 4, 5, 6, 7, 8, 9, 10, 11, 12, 13, 14, 15, 16, 17, or 18 of the foregoing targets

### Control Regions

It can be useful to include control regions to facilitate data validation. In some embodiments, the probes specific for the epigenetic target region set include probes specific for control methylated regions that are expected to be methylated in essentially all samples. In some embodiments, the probes specific for the epigenetic target region set include probes specific for control hypomethylated regions that are expected to be hypomethylated in essentially all samples.

### Probes Specific for Sequence-Variable Target Regions

The probes for the sequence-variable target region set may comprise probes specific for a plurality of regions known to undergo somatic mutations in cancer. The probes may be specific for any sequence-variable target region set described herein. Exemplary sequence-variable target region sets are discussed in detail herein, e.g., in the sections above concerning captured sets. [0366] In some embodiments, the sequence-variable target region probe set has a footprint of at least 0.5 kb, e.g., at least 1 kb, at least 2 kb, at least 5 kb, at least 10 kb, at least 20 kb, at least 30 kb, or at least 40 kb. In some embodiments, the epigenetic target region probe set has a footprint in the range of 0.5-100 kb, e.g., 0.5-2 kb, 2-10 kb, 10-20 kb, 20-30 kb, 30-40 kb, 40-50 kb, 50-60 kb, 60-70 kb, 70-80 kb, 80-90 kb, and 90-100 kb.

In some embodiments, probes specific for the sequence-variable target region set comprise probes specific for at least a portion of at least 5, at least 10, at least 15, at least 20, at least 25, at least 30, at least 35, at least 40, at least 45, at least 50, at least 55, at least 60, at least 65, or at 70 of the genes of Table 4. In some embodiments, probes specific for the sequence-variable target region set comprise probes specific for the at least 5, at least 10, at least 15, at least 20, at least 25, at least 30, at least 35, at least 40, at least 45, at least 50, at least 55, at least 60, at least 65, or 70 of the SNVs of Table 3. In some embodiments, probes specific for the sequence-variable target region set comprise probes specific for at least 1, at least 2, at least 3, at least 4, at least 5, or 6 of the fusions of Table 3. In some embodiments, probes specific for the sequence-variable target region set comprise probes specific for at least a portion of at least 1, at least 2, or 3 of the indels of Table 4. In some embodiments, probes specific for the sequence-variable target region set comprise probes specific for at least a portion of at least 5, at least 10, at least 15, at least 20, at least 25, at least 30, at least 35, at least 40, at least 45, at least 50, at least 55, at least 60, at least 65, at least 70, or 73 of the genes of Table 5. In some embodiments, probes specific for the sequence-variable target region set comprise probes specific for at least 5, at least 10, at least 15, at least 20, at least 25, at least 30, at least 35, at least 40, at least 45, at least 50, at least 55, at least 60, at least 65, at least 70, or 73 of the SNVs of Table 5. In some embodiments, probes specific for the sequence-variable target region set comprise probes specific for at least 1, at least 2, at least 3, at least 4, at least 5, or 6 of the fusions of Table 5. In some embodiments, probes specific for the sequence-variable target region set comprise probes specific for at least a portion of at least 1, at least 2, at least 3, at least 4, at least 5, at least 6, at least 7, at least 8, at least 9, at least 10, at least 11, at least 12, at least 13, at least 14, at least 15, at least 16, at least 17, or 18 of the indels of Table 5. In some embodiments, probes specific for the sequence-variable target region set comprise probes specific for at least a portion of at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, or at least 20 of the genes of Table 6.

In some embodiments, the probes specific for the sequence-variable target region set comprise probes specific for target regions from at least 10, 20, 30, or 35 cancer-related genes, such as AKTI, ALK, BRAF, CCND1, CDK2A, CTNNB1, EGFR, ERBB2, ESR1, FGFR1, FGFR2, FGFR3, FOXL2, GAT A3, GNA11, GNAQ, GNAS, HRAS, IDH1, IDH2, KIT, KRAS, MED 12, MET, MYC, NFE2L2, NRAS, PDGFRA, PIK3CA, PPP2R1A, PTEN, RET, STK11, TP53, and U2AF 1.

### Precision Treatments

The precision diagnostics provided by the improved computer system 110 may result in precision treatment plans, which may be identified by the computer system 110 (and/or curated by health professionals). For example, one type of precision diagnostic and treatment may relate to genes in the homologous recombination repair (HRR) pathway.

Homologous recombination is a type of genetic recombination in which nucleotide sequences are exchanged between two similar or identical molecules of DNA. It is most widely used by cells to accurately repair harmful breaks that occur on both strands of DNA, known as double-strand breaks (DSB). HRR provides a mechanism for the error-free removal of damage present in DNA that has replicated (S and G2 phases), to eliminate chromosomal breaks before the cell division occurs. The primary model for how homologous recombination repairs double-strand breaks in DNA is homologous recombination repair pathway which mediates the double-strand break repair (DSBR) pathway and the synthesis-dependent strand annealing (SDSA) pathway. Germline and somatic deficiencies in homologous recombination genes have been strongly linked to breast, ovarian and prostate cancers.

The number and types of variant nucleotides in a sample can provide an indication of the amenability of the subject providing the sample to treatment, i.e., therapeutic intervention. For example, various poly ADP ribose polymerase (PARP) inhibitors have been shown to stop the growth of tumors from breast, ovarian and prostate cancers caused by hereditary mutations in the BRCA1 or BRCA2 genes. Some of these therapeutic agents may inhibit base excision repair (BER), which may compensate for the deficiency of HRR.

On the other hand, certain BRCA and HRR wildtype patients may not achieve clinical benefit from treatment with a PARP inhibitor. Furthermore, not all ovarian cancer patients with a BRCA mutation will respond to a PARP inhibitor. Moreover, different types of mutations may indicate different therapies. For example, somatic heterozygous deletions in HRR genes may indicate a different therapy than somatic homozygous deletions. Thus, the state of genetic material may influence therapy. In one example, a PARP inhibitor may be administered to an individual harboring a somatic homozygous deletion in a HRR gene, but not to an individual harboring a wildtype allele or somatic heterozygous deletions in the HRR gene.

In some implementations, a subject having HRD as determined by any of the methods disclosed may be administered a targeted therapy. The targeted therapy may comprise a PARP inhibitor. Examples of PARP inhibitors that may be administered include one or more of: VELIPARIB, OLAPARIB, TALAZOPARIB, RUCAPARIB, NIRAPARIB, PAMIPARIB, CEP 9722 (Cephalon), E7016 (Eisai), E7449 (Eisai, a PARP ½ and tankyrase ½ inhibitor), or 3-Aminobenzamide. In some implementations, the targeted therapy may comprise at least one base excision repair (BER) inhibitor. For example, OLAPARIB may inhibit BER. In certain implementations, the targeted therapy may comprise combination of a PARP inhibitor and radiotherapy. In an implementation, the combination of a PARP inhibitor and radiotherapy would permit the PARP inhibitor to lead to formation of double strand breaks from the single-strand breaks generated by the radiotherapy in tumor tissue (e.g., tissue with BRCA1/BRCA2 mutations). This combination can provide more powerful therapy per radiation dose.

### Customized Therapies and Related Administrations

In some implementations, the methods disclosed herein relate to identifying and administering therapies to patients having a given disease, disorder or condition. Essentially any cancer therapy (e.g., surgical therapy, radiation therapy, chemotherapy, and/or the like) is included as part of these methods. In certain implementations, the therapy administered to a subject may comprise at least one chemotherapy drug. In some implementations, the chemotherapy drug may comprise alkylating agents (for example, but not limited to, Chlorambucil, Cyclophosphamide, Cisplatin and Carboplatin), nitrosoureas (for example, but not limited to, Carmustine and Lomustine), anti-metabolites (for example, but not limited to, Fluorauracil, Methotrexate and Fludarabine), plant alkaloids and natural products (for example, but not limited to, Vincristine, Paclitaxel and Topotecan), anti-tumor antibiotics (for example, but not limited to, Bleomycin, Doxorubicin and Mitoxantrone), hormonal agents (for example, but not limited to, Prednisone, Dexamethasone, Tamoxifen and Leuprolide) and biological response modifiers (for example, but not limited to, Herceptin and Avastin, Erbitux and Rituxan). In some implementations, the chemotherapy administered to a subject may comprise FOLFOX or FOLFIRI. Typically, therapies include at least one immunotherapy (or an immunotherapeutic agent). Immunotherapy refers generally to methods of enhancing an immune response against a given cancer type. In certain implementations, immunotherapy refers to methods of enhancing a T cell response against a tumor or cancer.

In some implementations, the immunotherapy or immunotherapeutic agents targets an immune checkpoint molecule. Certain tumors are able to evade the immune system by co-opting an immune checkpoint pathway. Thus, targeting immune checkpoints has emerged as an effective approach for countering a tumor's ability to evade the immune system and activating anti-tumor immunity against certain cancers. Pardoll, Nature Reviews Cancer, 2012, 12:252-264.

In certain implementations, the immune checkpoint molecule is an inhibitory molecule that reduces a signal involved in the T cell response to antigen. For example, CTLA4 is expressed on T cells and plays a role in downregulating T cell activation by binding to CD80 (aka B7.1) or CD86 (aka B7.2) on antigen presenting cells. PD-1 is another inhibitory checkpoint molecule that is expressed on T cells. PD-1 limits the activity of T cells in peripheral tissues during an inflammatory response. In addition, the ligand for PD-1 (PD-L1 or PD-L2) is commonly upregulated on the surface of many different tumors, resulting in the downregulation of anti-tumor immune responses in the tumor microenvironment. In certain implementations, the inhibitory immune checkpoint molecule is CTLA4 or PD-1. In other implementations, the inhibitory immune checkpoint molecule is a ligand for PD-1, such as PD-L1 or PD-L2. In other implementations, the inhibitory immune checkpoint molecule is a ligand for CTLA4, such as CD80 or CD86. In other implementations, the inhibitory immune checkpoint molecule is lymphocyte activation gene 3 (LAG3), killer cell immunoglobulin like receptor (KIR), T cell membrane protein 3 (TIM3), galectin 9 (GAL9), or adenosine A2a receptor (A2aR).

Antagonists that target these immune checkpoint molecules can be used to enhance antigen-specific T cell responses against certain cancers. Accordingly, in certain implementations, the immunotherapy or immunotherapeutic agent is an antagonist of an inhibitory immune checkpoint molecule. In certain implementations, the inhibitory immune checkpoint molecule is PD-1. In certain implementations, the inhibitory immune checkpoint molecule is PD-L1. In certain implementations, the antagonist of the inhibitory immune checkpoint molecule is an antibody (e.g., a monoclonal antibody). In certain implementations, the antibody or monoclonal antibody is an anti-CTLA4, anti-PD-1, anti-PD-L1, or anti-PD-L2 antibody. In certain implementations, the antibody is a monoclonal anti-PD-1 antibody. In some implementations, the antibody is a monoclonal anti-PD-L1 antibody. In certain implementations, the monoclonal antibody is a combination of an anti-CTLA4 antibody and an anti-PD-1 antibody, an anti-CTLA4 antibody and an anti-PD-L1 antibody, or an anti-PD-L1 antibody and an anti-PD-1 antibody. In certain implementations, the anti-PD-1 antibody is one or more of pembrolizumab (Keytruda®) or nivolumab (Opdivo®). In certain implementations, the anti-CTLA4 antibody is ipilimumab (Yervoy®). In certain implementations, the anti-PD-L1 antibody is one or more of atezolizumab (Tecentriq®), avelumab (Bavencio®), or durvalumab (Imfinzi®).

In certain implementations, the immunotherapy or immunotherapeutic agent is an antagonist (e.g. antibody) against CD80, CD86, LAG3, KIR, TIM3, GAL9, or A2aR. In other implementations, the antagonist is a soluble version of the inhibitory immune checkpoint molecule, such as a soluble fusion protein comprising the extracellular domain of the inhibitory immune checkpoint molecule and an Fc domain of an antibody. In certain implementations, the soluble fusion protein includes the extracellular domain of CTLA4, PD-1, PD-L1, or PD-L2. In some implementations, the soluble fusion protein includes the extracellular domain of CD80, CD86, LAG3, KIR, TIM3, GAL9, or A2aR. In one implementation, the soluble fusion protein includes the extracellular domain of PD-L2 or LAG3.

In certain implementations, the immune checkpoint molecule is a co-stimulatory molecule that amplifies a signal involved in a T cell response to an antigen. For example, CD28 is a co-stimulatory receptor expressed on T cells. When a T cell binds to antigen through its T cell receptor, CD28 binds to CD80 (aka B7.1) or CD86 (aka B7.2) on antigen-presenting cells to amplify T cell receptor signaling and promote T cell activation. Because CD28 binds to the same ligands (CD80 and CD86) as CTLA4, CTLA4 is able to counteract or regulate the co-stimulatory signaling mediated by CD28. In certain implementations, the immune checkpoint molecule is a co-stimulatory molecule selected from CD28, inducible T cell co-stimulator (ICOS), CD137, OX40, or CD27. In other implementations, the immune checkpoint molecule is a ligand of a co-stimulatory molecule, including, for example, CD80, CD86, B7RP1, B7-H3, B7-H4, CD137L, OX40L, or CD70.

Agonists that target these co-stimulatory checkpoint molecules can be used to enhance antigen-specific T cell responses against certain cancers. Accordingly, in certain implementations, the immunotherapy or immunotherapeutic agent is an agonist of a co-stimulatory checkpoint molecule. In certain implementations, the agonist of the co-stimulatory checkpoint molecule is an agonist antibody and preferably is a monoclonal antibody. In certain implementations, the agonist antibody or monoclonal antibody is an anti-CD28 antibody. In other implementations, the agonist antibody or monoclonal antibody is an anti-ICOS, anti-CD137, anti-OX40, or anti-CD27 antibody. In other implementations, the agonist antibody or monoclonal antibody is an anti-CD80, anti-CD86, anti-B7RP1, anti-B7-H3, anti-B7-H4, anti-CD137L, anti-OX40L, or anti-CD70 antibody.

Therapeutic options for treating specific genetic-based diseases, disorders, or conditions, other than cancer, are generally well-known to those of ordinary skill in the art and will be apparent given the particular disease, disorder, or condition under consideration.

In certain implementations, the customized therapies described herein are typically administered parenterally (e.g., intravenously or subcutaneously). Pharmaceutical compositions containing the immunotherapeutic agent are typically administered intravenously. Certain therapeutic agents are administered orally. However, customized therapies (e.g., immunotherapeutic agents, etc.) may also be administered by any method known in the art, including, for example, buccal, sublingual, rectal, vaginal, intraurethral, topical, intraocular, intranasal, and/or intraauricular, which administration may include tablets, capsules, granules, aqueous suspensions, gels, sprays, suppositories, salves, ointments, or the like.

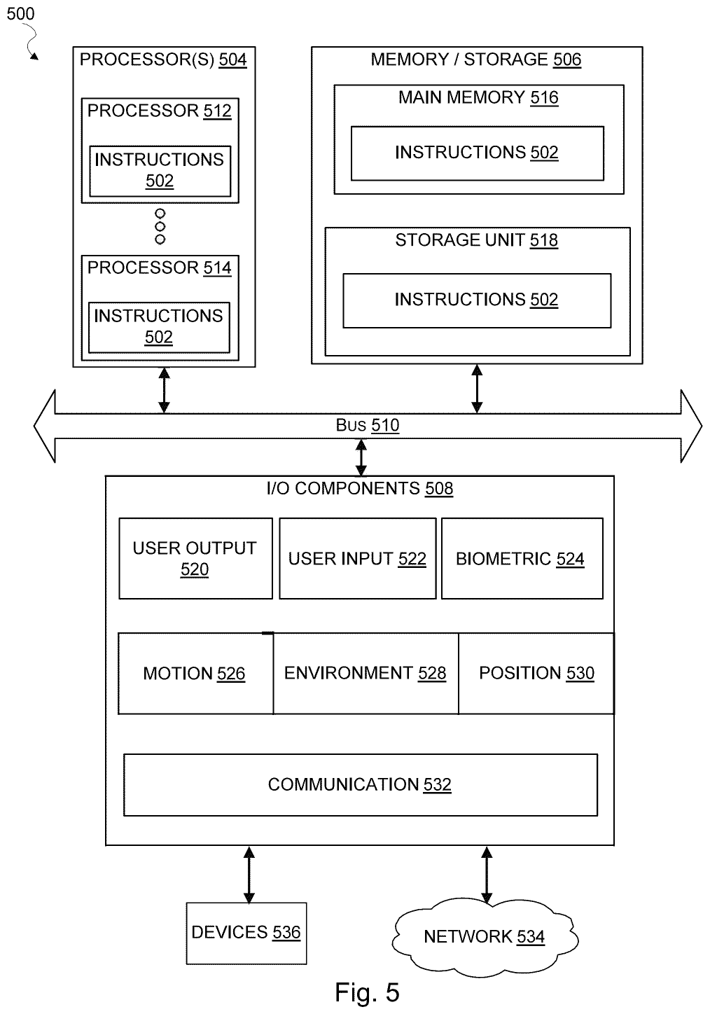

FIG. 5 is a block diagram illustrating components of a machine 500, according to some example implementations, able to read instructions from a machine-readable medium (e.g., a machine-readable storage medium) and perform any one or more of the methodologies discussed herein. Specifically, FIG. 5 shows a diagrammatic representation of the machine 500 in the example form of a computer system, within which instructions 502 (e.g., software, a program, an application, an applet, an app, or other executable code) for causing the machine 500 to perform any one or more of the methodologies discussed herein may be executed. As such, the instructions 502 may be used to implement modules or components described herein. The instructions 502 transform the general, non-programmed machine 500 into a particular machine 500 programmed to carry out the described and illustrated functions in the manner described. In alternative implementations, the machine 500 operates as a standalone device or may be coupled (e.g., networked) to other machines. In a networked deployment, the machine 500 may operate in the capacity of a server machine or a client machine in a server-client network environment, or as a peer machine in a peer-to-peer (or distributed) network environment. The machine 500 may comprise, but not be limited to, a server computer, a client computer, a personal computer (PC), a tablet computer, a laptop computer, a netbook, a set-top box (STB), a personal digital assistant (PDA), an entertainment media system, a cellular telephone, a smart phone, a mobile device, a wearable device (e.g., a smart watch), a smart home device (e.g., a smart appliance), other smart devices, a web appliance, a network router, a network switch, a network bridge, or any machine capable of executing the instructions 502, sequentially or otherwise, that specify actions to be taken by machine 500. Further, while only a single machine 500 is illustrated, the term “machine” shall also be taken to include a collection of machines that individually or jointly execute the instructions 502 to perform any one or more of the methodologies discussed herein.

The machine 500 may include processors 504, memory/storage 506, and I/O components 508, which may be configured to communicate with each other such as via a bus 510. In an example implementation, the processors 504 (e.g., a central processing unit (CPU), a reduced instruction set computing (RISC) processor, a complex instruction set computing (CISC) processor, a graphics processing unit (GPU), a digital signal processor (DSP), an application-specific integrated circuit (ASIC), a radio-frequency integrated circuit (RFIC), another processor, or any suitable combination thereof) may include, for example, a processor 512 and a processor 514 that may execute the instructions 502. The term “processor” is intended to include multi-core processors 504 that may comprise two or more independent processors (sometimes referred to as “cores”) that may execute instructions 502 contemporaneously. Although FIG. 5 shows multiple processors 504, the machine 500 may include a single processor 512 with a single core, a single processor 512 with multiple cores (e.g., a multi-core processor), multiple processors 512, 514 with a single core, multiple processors 512, 514 with multiple cores, or any combination thereof.

The memory/storage 506 may include memory, such as a main memory 516, or other memory storage, and a storage unit 518, both accessible to the processors 504 such as via the bus 510. The storage unit 518 and main memory 516 store the instructions 502 embodying any one or more of the methodologies or functions described herein. The instructions 502 may also reside, completely or partially, within the main memory 516, within the storage unit 518, within at least one of the processors 504 (e.g., within the processor's cache memory), or any suitable combination thereof, during execution thereof by the machine 500. Accordingly, the main memory 516, the storage unit 518, and the memory of processors 504 are examples of machine-readable media.

The I/O components 508 components 508 may include a wide variety of components to receive input, provide output, produce output, transmit information, exchange information, capture measurements, and so on. The specific I/O components 508 that are included in a particular machine 500 will depend on the type of machine. For example, portable machines such as mobile phones will likely include a touch input device or other such input mechanisms, while a headless server machine will likely not include such a touch input device. It will be appreciated that the I/O components 508 components 508 may include many other components that are not shown in FIG. 5. The I/O components 508 are grouped according to functionality merely for simplifying the following discussion and the grouping is in no way limiting. In various example implementations, the I/O components 508 components 508 may include user output components 520 and user input components 522. The user output components 520 may include visual components (e.g., a display such as a plasma display panel (PDP), a light emitting diode (LED) display, a liquid crystal display (LCD), a projector, or a cathode ray tube (CRT)), acoustic components (e.g., speakers), haptic components (e.g., a vibratory motor, resistance mechanisms), other signal generators, and so forth. The user input components 522 may include alphanumeric input components (e.g., a keyboard, a touch screen configured to receive alphanumeric input, a photo-optical keyboard, or other alphanumeric input components), point-based input components (e.g., a mouse, a touchpad, a trackball, a joystick, a motion sensor, or other pointing instrument), tactile input components (e.g., a physical button, a touch screen that provides location or force of touches or touch gestures, or other tactile input components), audio input components (e.g., a microphone), and the like.

In further example implementations, the I/O components 508 components 508 may include biometric components 524, motion components 526, environmental components 528, or position components 530 among a wide array of other components. For example, the biometric components 524 may include components to detect expressions (e.g., hand expressions, facial expressions, vocal expressions, body gestures, or eye tracking), measure biosignals (e.g., blood pressure, heart rate, body temperature, perspiration, or brain waves), identify a person (e.g., voice identification, retinal identification, facial identification, fingerprint identification, or electroencephalogram based identification), and the like. The motion components 526 may include acceleration sensor components (e.g., accelerometer), gravitation sensor components, rotation sensor components (e.g., gyroscope), and so forth. The environmental components 528 may include, for example, illumination sensor components (e.g., photometer), temperature sensor components (e.g., one or more thermometer that detect ambient temperature), humidity sensor components, pressure sensor components (e.g., barometer), acoustic sensor components (e.g., one or more microphones that detect background noise), proximity sensor components (e.g., infrared sensors that detect nearby objects), gas sensors (e.g., gas detection sensors to detection concentrations of hazardous gases for safety or to measure pollutants in the atmosphere), or other components that may provide indications, measurements, or signals corresponding to a surrounding physical environment. The position components 530 may include location sensor components (e.g., a GPS receiver component), altitude sensor components (e.g., altimeters or barometers that detect air pressure from which altitude may be derived), orientation sensor components (e.g., magnetometers), and the like.

Communication may be implemented using a wide variety of technologies. The I/O components 508 may include communication components 532 operable to couple the machine 500 to a network 534 or devices 536. For example, the communication components 532 may include a network interface component or other suitable device to interface with the network 534. In further examples, communication components 532 may include wired communication components, wireless communication components, cellular communication components, near field communication (NFC) components, Bluetooth® components (e.g., Bluetooth® Low Energy), Wi-Fi® components, and other communication components to provide communication via other modalities. The devices 536 may be another machine 500 or any of a wide variety of peripheral devices (e.g., a peripheral device coupled via a USB).

Moreover, the communication components 532 may detect identifiers or include components operable to detect identifiers. For example, the communication components 532 may include radio frequency identification (RFID) tag reader components, NFC smart tag detection components, optical reader components (e.g., an optical sensor to detect one-dimensional bar codes such as Universal Product Code (UPC) bar code, multi-dimensional bar codes such as Quick Response (QR) code, Aztec code, Data Matrix, Dataglyph, MaxiCode, PDF417, Ultra Code, UCC RSS-2D bar code, and other optical codes), or acoustic detection components (e.g., microphones to identify tagged audio signals). In addition, a variety of information may be derived via the communication components 532, such as location via Internet Protocol (IP) geo-location, location via Wi-Fi® signal triangulation, location via detecting an NFC beacon signal that may indicate a particular location, and so forth.

As used herein, “component” refers to a device, physical entity, or logic having boundaries defined by function or subroutine calls, branch points, APIs, or other technologies that provide for the partitioning or modularization of particular processing or control functions. Components may be combined via their interfaces with other components to carry out a machine process. A component may be a packaged functional hardware unit designed for use with other components and a part of a program that usually performs a particular function of related functions. Components may constitute either software components (e.g., code embodied on a machine-readable medium) or hardware components. A “hardware component” is a tangible unit capable of performing certain operations and may be configured or arranged in a certain physical manner. In various example implementations, one or more computer systems (e.g., a standalone computer system, a client computer system, or a server computer system) or one or more hardware components of a computer system (e.g., a processor or a group of processors) may be configured by software (e.g., an application or application portion) as a hardware component that operates to perform certain operations as described herein.

A hardware component may also be implemented mechanically, electronically, or any suitable combination thereof. For example, a hardware component may include dedicated circuitry or logic that is permanently configured to perform certain operations. A hardware component may be a special-purpose processor, such as a field-programmable gate array (FPGA) or an ASIC. A hardware component may also include programmable logic or circuitry that is temporarily configured by software to perform certain operations. For example, a hardware component may include software executed by a general-purpose processor 504 or other programmable processor. Once configured by such software, hardware components become specific machines (or specific components of a machine 500) uniquely tailored to perform the configured functions and are no longer general-purpose processors 504. It will be appreciated that the decision to implement a hardware component mechanically, in dedicated and permanently configured circuitry, or in temporarily configured circuitry (e.g., configured by software) may be driven by cost and time considerations. Accordingly, the phrase “hardware component” (or “hardware-implemented component”) should be understood to encompass a tangible entity, be that an entity that is physically constructed, permanently configured (e.g., hardwired), or temporarily configured (e.g., programmed) to operate in a certain manner or to perform certain operations described herein. Considering implementations in which hardware components are temporarily configured (e.g., programmed), each of the hardware components need not be configured or instantiated at any one instance in time. For example, where a hardware component comprises a general-purpose processor 504 configured by software to become a special-purpose processor, the general-purpose processor 504 may be configured as respectively different special-purpose processors (e.g., comprising different hardware components) at different times. Software accordingly configures a particular processor 512 processor 512, 514 or processors 504, for example, to constitute a particular hardware component at one instance of time and to constitute a different hardware component at a different instance of time.

Hardware components can provide information to, and receive information from, other hardware components. Accordingly, the described hardware components may be regarded as being communicatively coupled. Where multiple hardware components exist contemporaneously, communications may be achieved through signal transmission (e.g., over appropriate circuits and buses) between or among two or more of the hardware components. In implementations in which multiple hardware components are configured or instantiated at different times, communications between such hardware components may be achieved, for example, through the storage and retrieval of information in memory structures to which the multiple hardware components have access. For example, one hardware component may perform an operation and store the output of that operation in a memory device to which it is communicatively coupled. A further hardware component may then, at a later time, access the memory device to retrieve and process the stored output.

Hardware components may also initiate communications with input or output devices, and can operate on a resource (e.g., a collection of information). The various operations of example methods described herein may be performed, at least partially, by one or more processors 504 that are temporarily configured (e.g., by software) or permanently configured to perform the relevant operations. Whether temporarily or permanently configured, such processors 504 may constitute processor-implemented components that operate to perform one or more operations or functions described herein. As used herein, “processor-implemented component” refers to a hardware component implemented using one or more processors 504. Similarly, the methods described herein may be at least partially processor-implemented, with a particular processor 512 processor 512, 514 or processors 504 being an example of hardware. For example, at least some of the operations of a method may be performed by one or more processors 504 or processor-implemented components. Moreover, the one or more processors 504 may also operate to support performance of the relevant operations in a “cloud computing” environment or as a “software as a service” (SaaS). For example, at least some of the operations may be performed by a group of computers (as examples of machines 1000 including processors 504), with these operations being accessible via a network 534 (e.g., the Internet) and via one or more appropriate interfaces (e.g., an API). The performance of certain of the operations may be distributed among the processors, not only residing within a single machine 500, but deployed across a number of machines. In some example implementations, the processors 504 or processor-implemented components may be located in a single geographic location (e.g., within a home environment, an office environment, or a server farm). In other example implementations, the processors 504 or processor-implemented components may be distributed across a number of geographic locations.

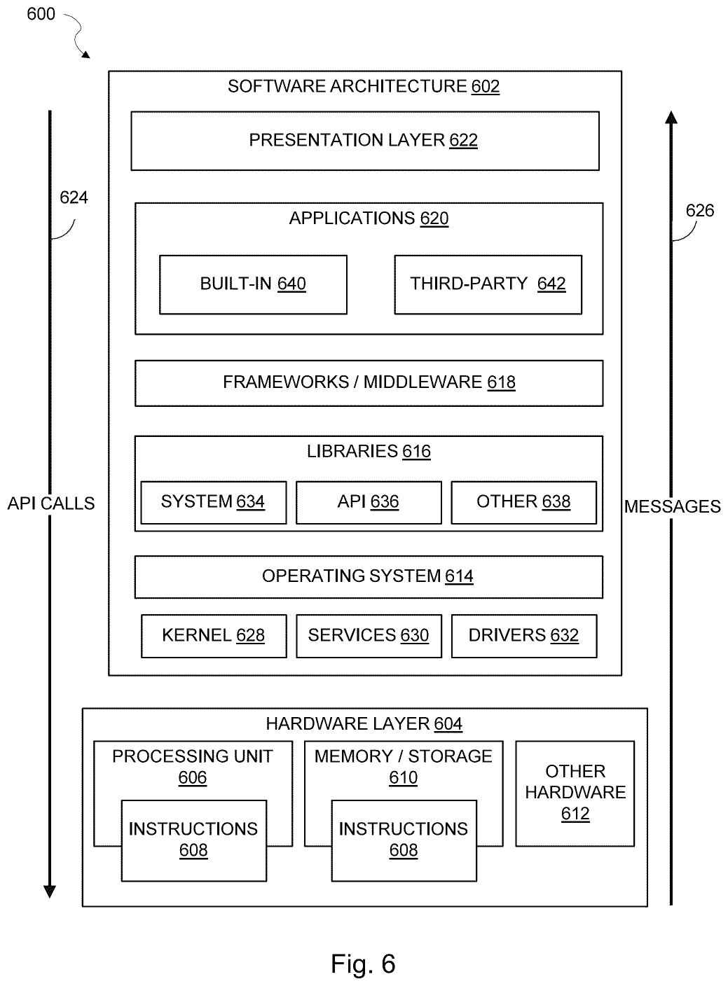

FIG. 6 is a block diagram illustrating system 600 that includes an example software architecture 602, which may be used in conjunction with various hardware architectures herein described. FIG. 6 is a non-limiting example of a software architecture, and it will be appreciated that many other architectures may be implemented to facilitate the functionality described herein. The software architecture 602 may execute on hardware such as machine 500 of FIG. 5 that includes, among other things, processors 504, memory/storage 506, and input/output (I/O) components 508. A representative hardware layer 604 is illustrated and can represent, for example, the machine 500 of FIG. 5. The representative hardware layer 604 includes a processing unit 606 having associated executable instructions 608. Executable instructions 608 represent the executable instructions of the software architecture 602, including implementation of the methods, components, and so forth described herein. The hardware layer 604 also includes at least one of memory or storage modules memory/storage 610, which also have executable instructions 608. The hardware layer 604 may also comprise other hardware 612.

In the example architecture of FIG. 6, the software architecture 602 may be conceptualized as a stack of layers where each layer provides particular functionality. For example, the software architecture 602 may include layers such as an operating system 614, libraries 616, frameworks/middleware 618, applications 620, and a presentation layer 622. Operationally, the applications 620 or other components within the layers may invoke API calls 624 through the software stack and receive messages 626 in response to the API calls 624. The layers illustrated are representative in nature and not all software architectures have all layers. For example, some mobile or special purpose operating systems may not provide a frameworks/middleware 618, while others may provide such a layer. Other software architectures may include additional or different layers.

The operating system 614 may manage hardware resources and provide common services. The operating system 614 may include, for example, a kernel 628, services 630, and drivers 632. The kernel 628 may act as an abstraction layer between the hardware and the other software layers. For example, the kernel 628 may be responsible for memory management, processor management (e.g., scheduling), component management, networking, security settings, and so on. The services 630 may provide other common services for the other software layers. The drivers 632 are responsible for controlling or interfacing with the underlying hardware. For instance, the drivers 632 include display drivers, camera drivers, Bluetooth® drivers, flash memory drivers, serial communication drivers (e.g., Universal Serial Bus (USB) drivers), Wi-Fi® drivers, audio drivers, power management drivers, and so forth depending on the hardware configuration.

The libraries 616 provide a common infrastructure that is used by at least one of the applications 620, other components, or layers. The libraries 616 provide functionality that allows other software components to perform tasks in an easier fashion than to interface directly with the underlying operating system 614 functionality (e.g., kernel 628, services 630, drivers 632). The libraries 616 may include system libraries 634 (e.g., C standard library) that may provide functions such as memory allocation functions, string manipulation functions, mathematical functions, and the like. In addition, the libraries 616 may include API libraries 636 such as media libraries (e.g., libraries to support presentation and manipulation of various media format such as MPEG4, H.264, MP3, AAC, AMR, JPG, PNG), graphics libraries (e.g., an OpenGL framework that may be used to render two-dimensional and three-dimensional in a graphic content on a display), database libraries (e.g., SQLite that may provide various relational database functions), web libraries (e.g., WebKit that may provide web browsing functionality), and the like. The libraries 616 may also include a wide variety of other libraries 638 to provide many other APIs to the applications 620 and other software components/modules.

The frameworks/middleware 618 (also sometimes referred to as middleware) provide a higher-level common infrastructure that may be used by the applications 620 or other software components/modules. For example, the frameworks/middleware 618 may provide various graphical user interface functions, high-level resource management, high-level location services, and so forth. The frameworks/middleware 618 may provide a broad spectrum of other APIs that may be utilized by the applications 620 or other software components/modules, some of which may be specific to a particular operating system 614 or platform.

The applications 620 include built-in applications 640 and third-party applications 642. Examples of representative built-in applications 640 may include, but are not limited to, a contacts application, a browser application, a book reader application, a location application, a media application, a messaging application, or a game application. Third-party applications 642 may include an application developed using the ANDROID™ or IOS™ software development kit (SDK) by an entity other than the vendor of the particular platform, and may be mobile software running on a mobile operating system such as IOS™, ANDROID™, WINDOWS® Phone, or other mobile operating systems. The third-party applications 642 may invoke the API calls 624 provided by the mobile operating system (such as operating system 614) to facilitate functionality described herein.

The applications 620 may use built-in operating system functions (e.g., kernel 628, services 630, drivers 632), libraries 616, and frameworks/middleware 618 to create UIs to interact with users of the system. Alternatively, or additionally, in some systems, interactions with a user may occur through a presentation layer, such as presentation layer 622. In these systems, the application/component “logic” can be separated from the aspects of the application/component that interact with a user.

At least some of the processes described herein can be embodied in computer-readable instructions for execution by one or more processors such that the operations of the processes may be performed in part or in whole by the functional components of one or more computer systems. Accordingly, computer-implemented processes described herein are by way of example with reference thereto, in some situations. However, in other implementations, at least some of the operations of the computer-implemented processes described herein can be deployed on various other hardware configurations. The computer-implemented processes described herein are therefore not intended to be limited to the systems and configurations described with respect to FIGS. 5 and 6 and can be implemented in whole, or in part, by one or more additional system and/or components.

Although the flowcharts described herein can show operations as a sequential process, many of the operations can be performed in parallel or concurrently. In addition, the order of the operations can be re-arranged. A process is terminated when its operations are completed. A process can correspond to a method, a procedure, an algorithm, etc. The operations of methods may be performed in whole or in part, can be performed in conjunction with some or all of the operations in other methods, and can be performed by any number of different systems, such as the systems described herein, or any portion thereof, such as a processor included in any of the systems.

### EXAMPLES

### Example 1—Genomic and Epigenomic Detection Assay

In this analysis, the Inventors demonstrated our highly sensitive targeted assay that simultaneously captures both genomic alterations and methylation signatures in cell-free DNA (cfDNA). Our assay can detect differential methylations that classify cancer from healthy donors, as well as the quantification of promoter methylation. To capture tumor-associated methylated cfDNA signals, the Inventors developed a custom assay on a broad genomic panel (˜15.2 Mb) targeting unmethylated regions in plasma cfDNA from healthy individuals. This panel covers promoter regions of 11,950 genes, including well-known tumor suppressor genes (TSGs) e.g., PTEN, TP53 and homologous recombination and repair (HRR) genes, e.g., BRCA1, RAD51.

For each sample that runs through the assay, with the pre-defined promoter regions of each covered gene, a methylation score is derived from observed methylation signatures. To train the method of making promoter hyper-methylation calls, the Inventors first trained and evaluated the specificity of the model on blood samples of 131 cancer-free donor. The Inventors then tested the performance on a validation dataset of blood samples from 559 cancer patients (203 lung cancer, 146 breast cancer, 151 bladder cancer, 32 CRC and 27 other cancer types) and 2,631 cancer-free donors.

In blood samples of these 131 cancer-free donors, the Inventors established the threshold of making promoter-methylation calls. The Inventors focus our analysis on 88 cancer-related genes.

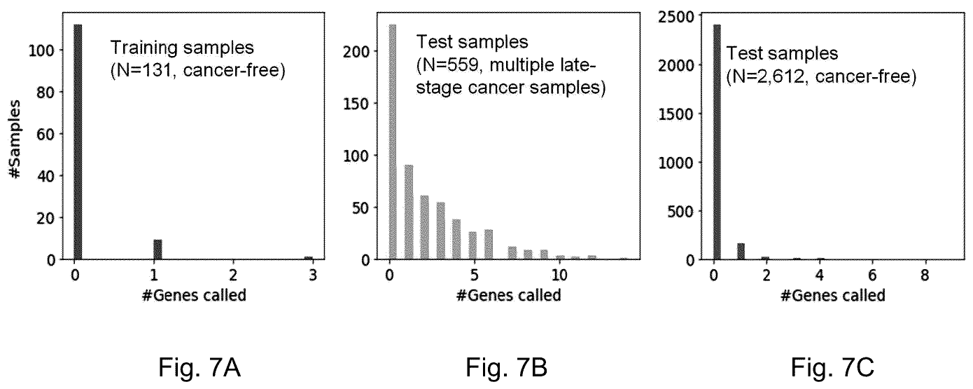

Under our established threshold, 12 promoter methylations (in at least one of the 88 genes) was observed across all 131 training samples (FIG. 7A). In the test dataset of 2,612 cancer-free donors, the Inventors observed a total of 251 promoter methylation calls in these 88 genes (FIG. 7C)—from that the specificity is estimated >99%. In the test data of 559 late-stage cancer patients, 334 (60%) were called with at least one promoter methylation in these genes. With samples that get promoter methylation calls, the median calls per samples is 3 (FIG. 7B).

The Inventors manually examined the distribution of methylation signals in cancer patients and healthy donors from the test data. In cancer patients with methylation calls, the Inventors observed signals mostly within CpG clusters. In most cancer-free donors, the Inventors observed no signals associated with methylation in this promoter region: e.g., in MLH1 promoter region, the Inventors didn't observe any methylation signals in the 131 healthy-donors from the training set.

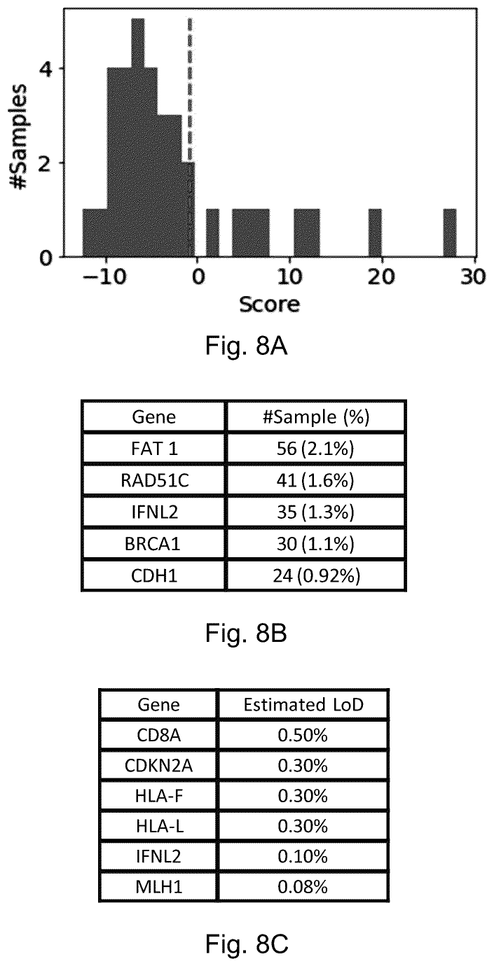

To further investigate the supposed false positives (FP), the Inventors examined cancer-prediction scores for cancer-free that have more than one methylation call. The Inventors found 40% have high cancer-prediction scores (>10 times of standard deviation than the cancer-normal cutoff, FIG. 8A). As “cancer-free” donors are self-identified in this cohort, it is likely these two donors have already developed cancer. FIG. 8B is a table showing genest that are called most often for promoter methylation in cancer-free donors.

For samples with low cancer-prediction scores, the Inventors also found likely true methylations for a few cancer-related genes, e.g. BRCA1. Publications indicate that promoter methylations may happen sporadically in the general population at a very low rate.

The Inventors calculated LoD of our method by in-silico mixing KM12 cell line and cancer-free donors at the level of 0.1%, 0.3%, 0.5%, 0.7% and 1%. The Inventors calculated LoD for 6 of the cancer-related genes that was called as promoter hyper-methylation with high signals in the cell line KM12 (FIG. 8C).

For each gene, its LoD is affected by methylation levels and its background noise in the healthy population. MLH1 with a fully methylation in KM12 (validated by orthogonal experiments) and has a very low methylation rate in the general population, has the lowest LoD among all genes. For the other 5 genes, their LoD ranges between 0.1% and 0.5%.

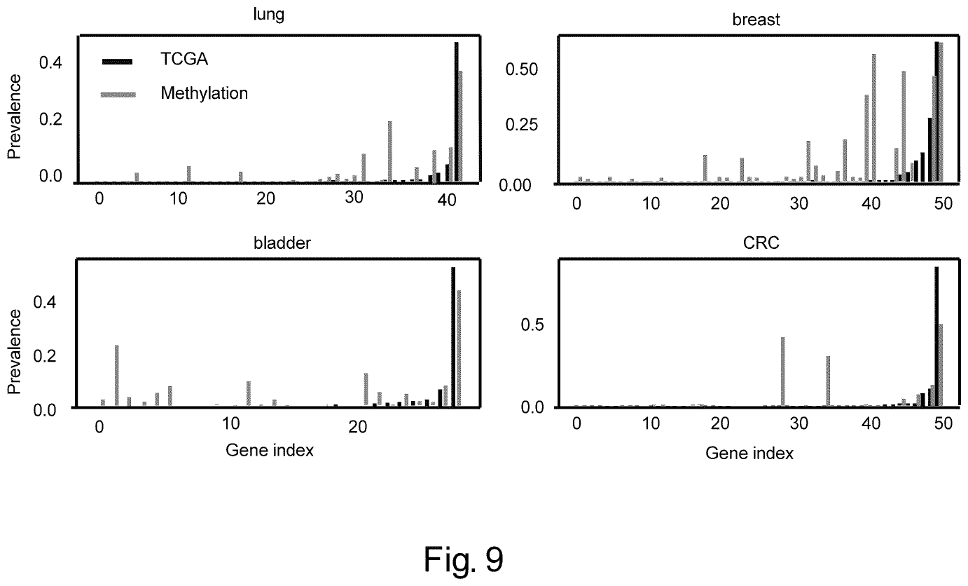

To further validate the promoter hyper-methylation and to validate our findings, the Inventors examined the TSG and HRR genes in cancer samples from the TCGA database. The Inventors focus our analysis on 2,380 primary tumor cancer samples with available methylation data from 450K array. For each gene, the Inventors calculated its prevalence (percentage of samples observed with promoter methylation) in both TCGA data and our test dataset (FIG. 9).

For most genes the Inventors observed similar prevalence between TCGA and our methylation detection method. It is worthwhile to note that samples in TCGA are tissue samples and many of them are still in early-stage, but our test dataset is mostly cfDNA from blood of late-stage patients.

The Inventors additionally examined the two genes (CD8A and CDKN2A) with high prevalence in our CRC samples but not in TCGA CRC data. The CD8A promoter region was covered by five array probes and two of them show >0.5 prevalence but this high prevalence was lowered by the other three array probes that show no methylation signals. A similar situation happens with CDKN2A which has 1 out of 5 array probes showing a prevalence of 0.1 but the other 4 have very low level of methylation signals.

To showcase the feasibility of our assay, the Inventors further summarized promoter methylation in MLH1. Previous studies have shown that 54%-100% of CRC patients with micro-satellite instability (MSI-H) tumor harbor MLH1 promoter methylation.

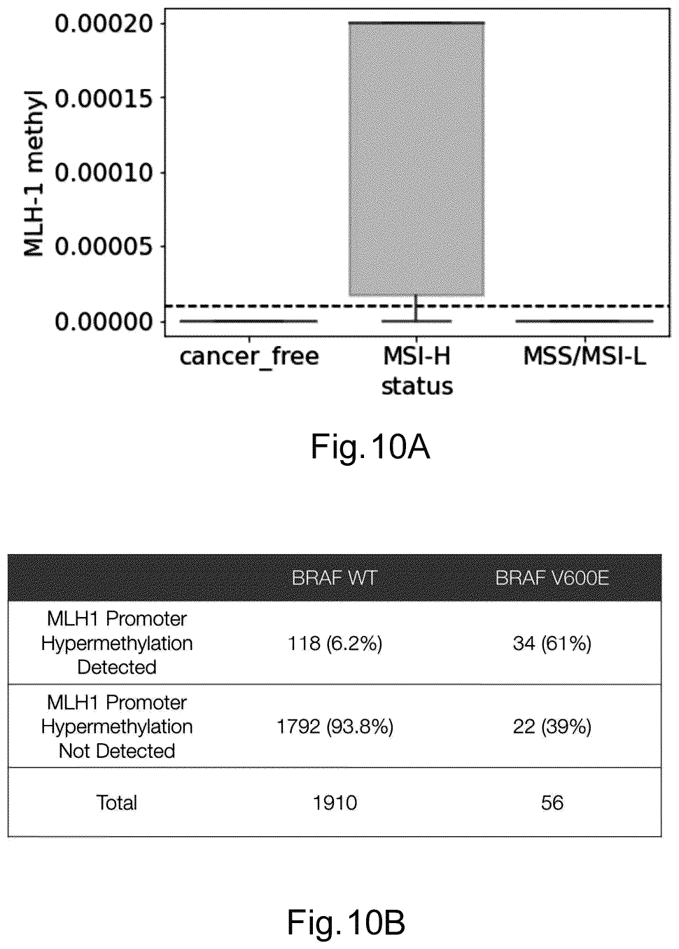

In 1,966 CRC patients, the Inventors detected significantly higher MLH1 promoter methylation in MSI-H group (Fisher's p<0.05), compared to patients with micro-satellite stable (MSS) status and the cancer-free population (FIG. 10A).

Furthermore, the Inventors tested the prevalence of the mutation, BRAF-V600E, a common driver mutation for CRC. Our finding shows the strong co-occurrence of MLH1 promoter hyper-methylation and BRAF-V600E (FIG. 10B), which is consistent with previous findings.

### Example 2—Liquid Biopsy

Liquid biopsy offers a rapid and non-invasive alternative to tissue biopsy for identifying biomarkers. More recently, its application has broadened to include assessment of early response to therapy (i.e. molecular response) and in the early-stage settings, detection of minimal residual disease (MRD) and early disease recurrence1. While circulating tumor fraction (cTF) estimated by somatic mutations is well associated with the tumor progression and prognosis, interference can occur from clonal hematopoiesis of indeterminate potential (CHIP), and for cell-free DNA (cfDNA) samples that lack detectable somatic mutations, somatic tumor fraction cannot be estimated. In this analysis, the Inventors demonstrate that epigenomic signatures accurately measure cTF using orthogonal analytes to somatic mutations and enable cTF estimation even in cases without detectable tumor driver variants.

To capture tumor-associated methylated cfDNA, the Inventors designed a custom assay on a broad genomic panel (15.2 Mb) that targets unmethylated regions in plasma cfDNA from healthy individuals. DNA molecules that support methylation were enriched by our assay and this information was post-processed into our machine learning model.

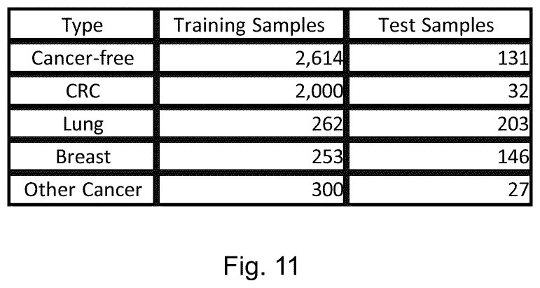

With this panel, the Inventors profiled plasma samples from a training set of ˜2,000 cancer patients and ˜2,600 cancer-free donors (FIG. 11). For the prediction of cancer/cancer-free status, the Inventors trained a logistic regression model. For tumor-fraction prediction, the Inventors trained a linear model utilizing the allele frequency of genomic variant calls as the underlying truth. The training performance was validated by 5-fold cross validation.

On the test dataset, the Inventors profiled 559 cancer patients 131 cancer-free donors. The Inventors applied all cancer-specific prediction models onto the test dataset to estimate 1) the cancer/cancer-free classification performance of single models and aggregated model; 2) the tumor fraction prediction performance.

To further benchmark the accuracy of our methylation models, the Inventors built an in-vitro and an in-silico titration datasets. The in-vitro titration dataset was generated by mixing cfDNA from patients with colorectal cancer (CRC) into the plasma from cancer-free donors via experimental titration. The in-silico titration dataset was generated by computationally mixing sequencing reads from CRC patients with those from cancer-free donors.

cTFs from methylated cfDNA may overcome the current limitations of somatic mutation-based methods. The Inventors demonstrate that our methylation approach is capable of accurately detecting cTFs in tumor-driver positive and negative cases. Our assay can reliably enrich molecules with methylation signals in differentially methylated signals in cancer patients. Our cancer-specific models achieves >90% detection rate for late-stage cancer patients while maintaining a 95% specificity. With genomic driver mutation calls as the surrogate for truth tumor fraction, our methylation-based prediction has a correlation of 0.85 with this surrogate on CRC.

As the Inventors estimate tumor-negative cases to be 30-50% of patients with stage I-III cancer and 15-20% of patients with stage IV cancer, our methylation approach may hold promise for providing better evaluation for patient care and management.

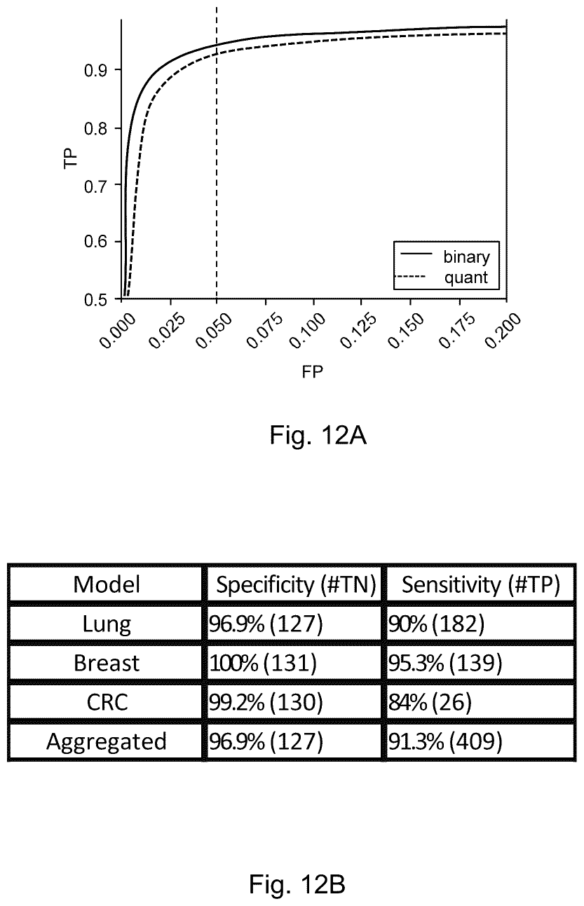

The Inventors built our machine-learning models for cancer/cancer-free classification and tumor-fraction prediction on the training dataset of 2,000 cancer patients and 2,614 cancer-free donors. The Inventors first evaluated our methods on the training dataset via five-fold cross validation—this process was repeated for 10 times. At 95% specificity, our prediction model for cancer/cancer-free status has an average of 93% detection rate for samples across all stages. The tumor-fraction prediction model has a similar performance as the status prediction model (FIG. 12A).

After the training process, the Inventors applied the trained models and their 95% specificity cutoff to the independent test dataset of 559 cancer patients and 131 cancer-free donors. On the test dataset, the Inventors observed a 97% specificity with a total of 4 false positives (FPs) observed across all three models. The FP in CRC model is included in the 4 FPs in the lung model (FIG. 12B).

The test dataset is all late-stage patients, the sensitivity of the breast cancer model (95%) is higher than the all-stage sensitivity (62%) in the training dataset where most (>70%) of its breast cancer patients are at early stage.

Manual examinations revealed that the FPs are slightly above the tumor-normal cutoff, as well as that some strong signals come from regions with sporadic mutations in cancer-free donors. It is possible that with advanced region definition and fine-tuning of the models, the FPs can be eliminated. On the other hand, a few FNs have relatively weaker signals in differentially methylated regions, indicating these samples might be less represented in the training data and addition of extra samples may help to increase the detection power.

The Inventors applied our CRC tumor-fraction prediction model onto the in-silico titration dataset of 1,000 samples, generated by randomly mixing reads from 1,000 CRC patient samples with 1,000 cancer-free donors. The method quantified a cTF over 0.1% in >99% of these samples. In contrast, when applied to 2,037 cancer-free donors, <5% of the samples resulted in an estimated cTFs >0.1%.

The Inventors applied our CRC tumor-fraction prediction model along with our genomic caller for CRC driver mutations on an in-vitro dataset comprised of 270 samples that were generated by experimentally titrating plasma from CRC patients into cancer-free donors at different levels.

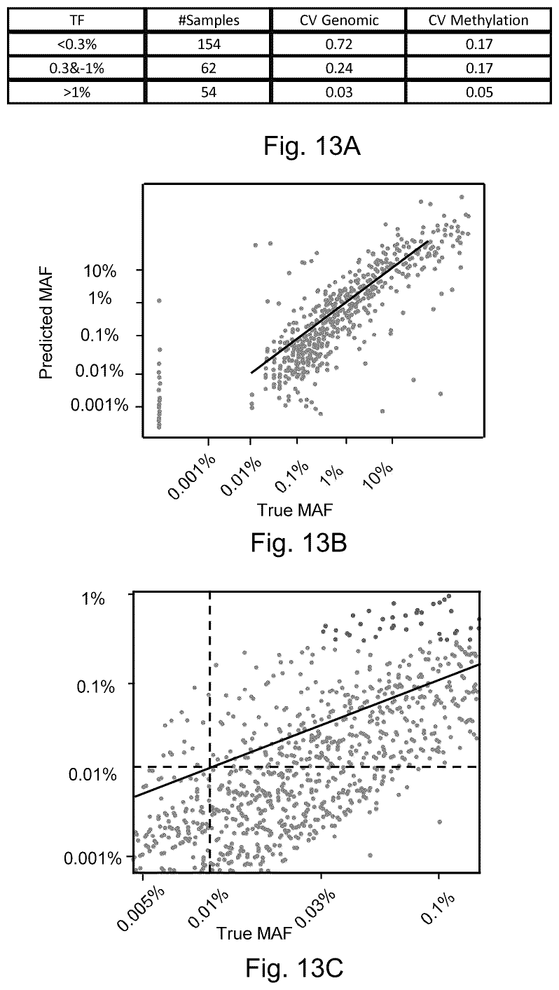

In all samples at low TF, the coefficient of variation (CV) of methylation was more robust than the CV from cTF estimated by genomic mutations: at TF of 0.3˜1%, methylation-based TF has a 5-fold lower than genomic TF (FIG. 13A).

With the predefined set of driver genes for CRC from previous studies, the Inventors used the allele frequency of driver mutations called from genomic data as the approximation for underlying true tumor fraction. The Inventors first compared the predicted tumor fraction from cross-validation against the true tumor fraction in the training set (FIG. 13B). With a Pearson correlation of 0.85, most of the methylation-based tumor fraction are consistent with the underlying truth.

For the top 10 outliers that have significantly higher methylation-predicted TF than the truth TF, 6 are cancer-free samples with high similarity to CRCs across our entire comprehensive panel. For the 19 outliers significantly lower methylation-predicted TF than the truth TF, it is possible that their extremely high genomic TF (>10%) comes from CHIP that escaped our driver-mutation filters.

It is possible that methylation can resolve samples with TF<0.1%; however, this possibility is difficult to evaluate as cTFx<0.1% are below the estimated limit of detection (LoD) for most of current companion diagnostic products. To test this out, the Inventors in-silico titrated molecules from 100 CRC patients into molecules from 100 cancer-free donors at the level between 0.005% and 0.01% and applied our trained models to this dataset (FIG. 13C).

While the cTFs of simulated samples in the 0.03%-0.01% range were consistent with the methylation-based cTF, estimates broke down below 0.01%. The Inventors hypothesize that below this level, there aren't enough true methylation signals existing above noise signals to allow for a robust cTF prediction.

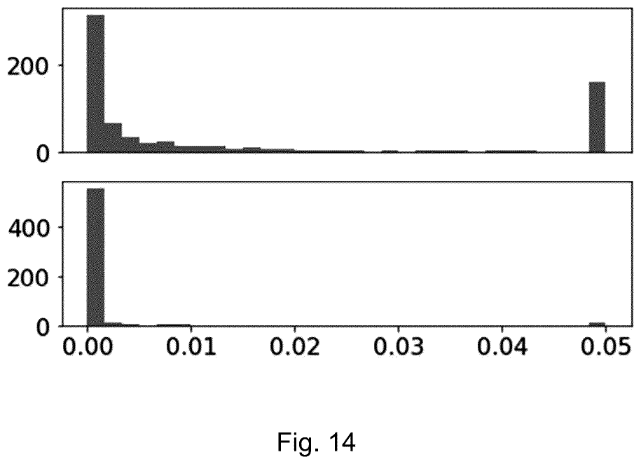

Previous studies have shown that 30-50% of patients with stage I-III cancer, and 15-20% of patients with stage IV cancer, may lack detectable driver mutations. Current methods relying on driver mutations thus cannot quantify the tumor fraction for these patients, leaving a large population with unmet need. Here, the Inventors demonstrated that methylation-based cTFs correlate strongly with mutation-based cTFs, thereby enabling the prediction of tumor fraction for this 15-50% of patients (FIG. 14).

As expected, the median predicted TF in these patients is lower than those with detectable driver mutations (0.3% vs 0.02%).

### Example 3.—CpG Methylation Analysis

Multi-cancer blood-based tests may yield clinical benefit in two ways: by improving adherence to guideline recommended cancer screening with a more convenient, easier to administer, and patient friendly modality, and by detection of early (stage I/II) tumors in cancer types that lack screening recommendations, yet early detection and intervention can save lives. A single test with clinically meaningful performance which addresses both opportunities has yet to be developed.

The Inventors evaluated a cfDNA device based on CpG methylation analysis that enables detection of colorectal (CRC), lung cancer, and multiple additional solid tumor cancer types with specificity thresholds tailored by cancer class based on current screening recommendations and clinical diagnostic pathways.

Methods. Blood samples were obtained from multiple cohorts of individuals with colorectal (N>2,000), lung (N >300), and other solid tumor cancers (bladder, gastric, liver, ovarian, pancreas (N>300)) as well as individuals without cancer (N>3,000).

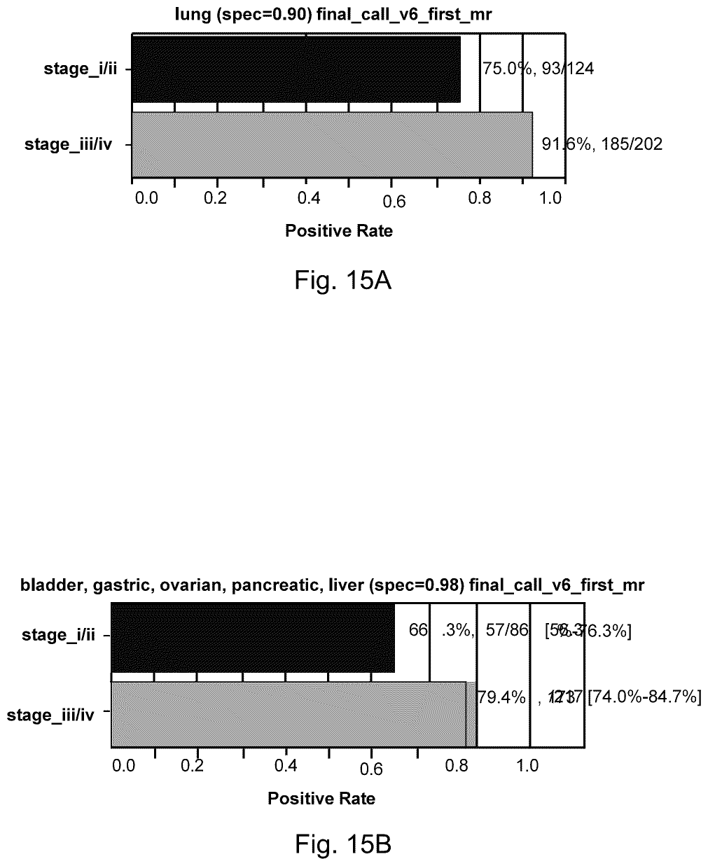

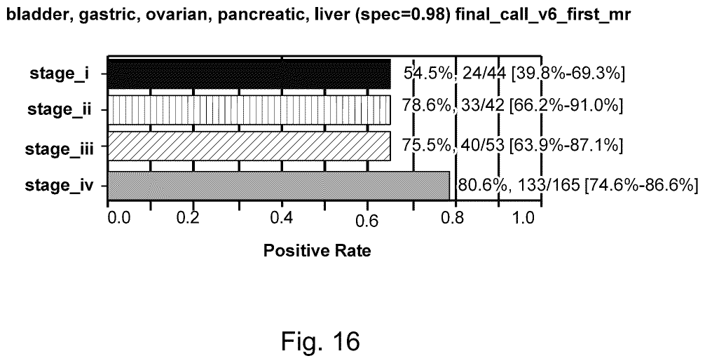

A liquid biopsy CRC screening test, which integrates genomics, epigenomics, and proteomics for the detection of cancer, is the backbone of the device. Lung and multi-cancer detection algorithms were developed through further analysis of the epigenomics patterns of the plasma derived cfDNA, which is enriched in the assay for fragments with high CpG density and high degree of methylation. Sensitivity for CRC and lung cancer detection is calculated at 90% target specificity thresholds. Multi-cancer specificity targeted a 98% threshold. Lung cancer and multi-cancer detection performances are obtained through combining the cross validation results from the development set and the results from a single pre-locked model on the validation set. FIG. 15A is a graphical representation showing positivity rates in individual for lung cancer detection in stage I/II patients and in page III/IV. patients. FIG. 15B is a graphical representation showing positivity rates in individuals for multi-cancer detection (bladder, gastric, ovarian, pancreatic, and liver) in stage I/II patients and in stage III/IV patients. Additionally, FIG. 16A is a graphical representation showing a more granular breakdown of positivity rates in individuals for multi-cancer detection (bladder, gastric, ovarian, pancreatic, and liver) in stage I patients, stage II patients, stage III patients, and stage IV patients. The specificity thresholds for CRC, lung, and multi-cancer are selected to yield assay performance tailored for the cancer type and clinical diagnostic pathway.

Results. This single device with tailored thresholds yielded CRC sensitivity of 91% (stage I/II: 93%) and lung cancer sensitivity of 85% (stage I/II: 75%) at 90% specificity. Multi-cancer sensitivity was 75% (stage I/II: 66%) at 98% specificity.

Conclusions. This blood-based cancer screening and detection device, with performance tailored to current cancer screening recommendations and clinical diagnostic pathways, yields performance on par with currently available screening tests for cancers with screening guidelines (CRC and lung) and clinically meaningful early-stage detection in cancer types without screening guidelines where early intervention can bring clinical benefit. With this profile, this multi-cancer blood-based test could cover 32% of the expected cancer diagnoses in 2022 according to SEER estimates, with 80% overall sensitivity (stage I/II: 78%), highlighting the ability of this technology to yield clinically meaningful results for the detection of early stage cancer.

### Example 4—Monitoring ctDNA Changes Over Time

Circulating tumor DNA (ctDNA) level and the change in level at a subsequent time point (e.g., on-treatment change from baseline or postoperative changes through time) are promising tools for predicting patient prognosis and response to therapy. Existing methods commonly use minor allele frequency (MAF) of somatic mutations to quantify circulating tumor fraction (cTF). Their performance can be limited by the number of detectable somatic mutations and the associated limit of detection (LoD), as well as interference from copy number variation and non-tumor alterations, such as clonal hematopoiesis (CHIP). Here, the Inventors describe the LoD, precision and limit of quantitation (LoQ) of cTF level and change using a next generation sequencing panel covering over 800 genes with genome-wide methylation detection.

The epigenomics cTF (represented by epiMAF) of a single sample is estimated from methylation signals across targeted regions of the methylation panel, calibrated using our internal training data that has clinical blood draw samples of over 5,000 individuals, including cancer-free donors and patients with mixed cancer types. Epigenomics cTF change compares two or more samples from the same patient to identify patient-specific methylated regions, and compare the methylation signals of the paired regions. Somatic mutations also were detected through the genomic panel.

LoD was determined as the minimum cTF level at which >=95% of replicates exhibited methylation signals derived from tumors. LoQ was defined as the minimum cTF level at which the coefficient of variation (CV) across replicates was less than 30%. The accuracy of methylation-based cTF was compared to cTFs derived from the maximum MAF of somatic mutations on 5,045 clinical samples of cancer patients.

One colorectal cancer sample, one breast cancer sample, one lung cancer sample, and one cell line sample were titrated into cancer-free backgrounds at target levels ranging from 0.1% to 0.5% MAF. The methylation LoD, which was defined as the lowest concentration of tumor-derived DNA detectable with >95% accuracy, was estimated to be approximately 0.05%.

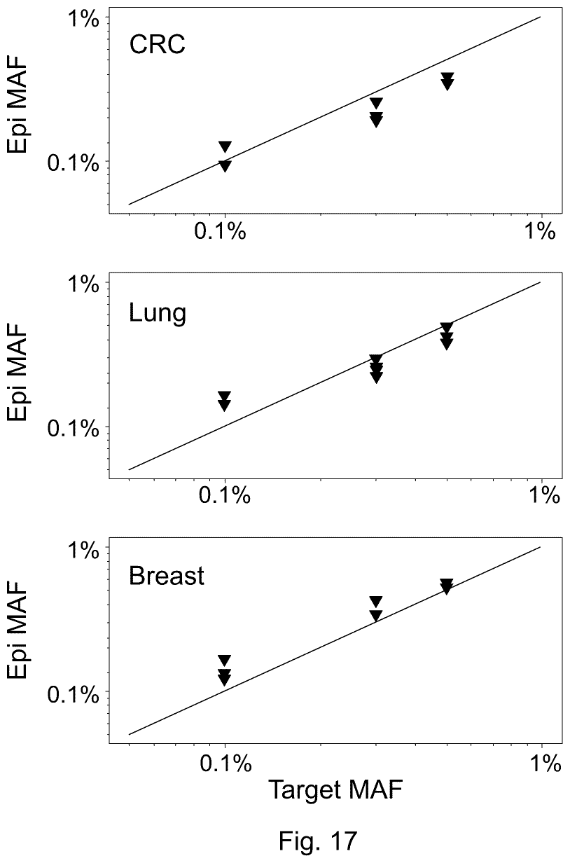

FIG. 17 is a graphical representation of epigenomic MAF in relation to target MAF for colorectal cancer, lung cancer, and breast cancer and indicates the accuracy of epigenomic cTF in clinical filtrations. The epigenomics cTF of clinical samples exhibit a high degree of consistency with underlying titration levels and maintain a strong linearity between different titration levels, as indicated by a Pearson-r of greater than 0.9 and a linearity error less than 5%.

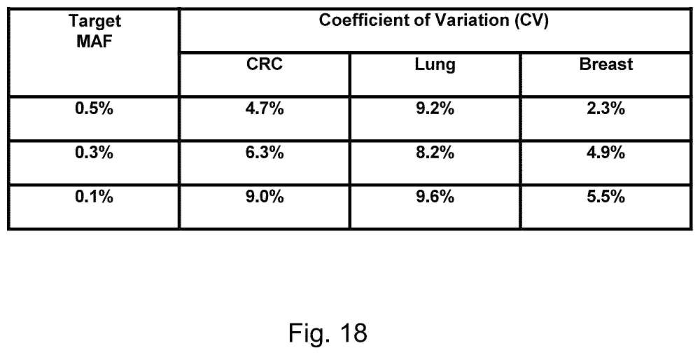

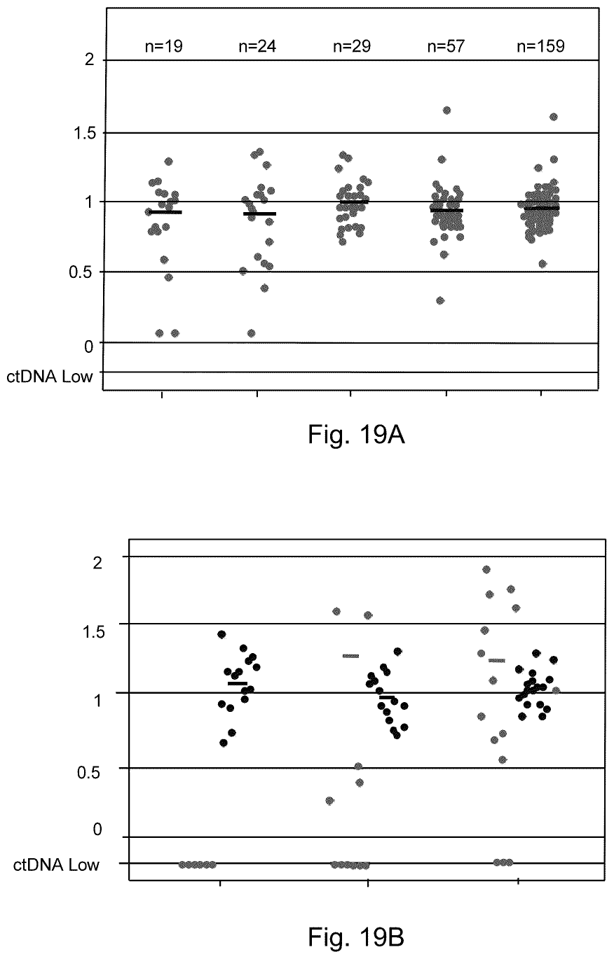

FIG. 18 is a table showing epigenomic cTF variations in technical replicates and indicates that the quantitative precision of epigenomics cTF is capable of reaching an LoQ of less than 0.1% in CRC, lung and breast clinical samples. FIG. 19A is a graphical representation showing that the somatic mutation based cTF is robust for replicates within the same cTF levels, particularly at cTF levels of 0.5% or higher. However, at lower titration levels, the epigenomic cTF is more stable. FIG. 19B is a graphical representation showing that the epigenomic cTF can maintain a 100% evaluation rate and has a LoQ down to 0.1% cTF.

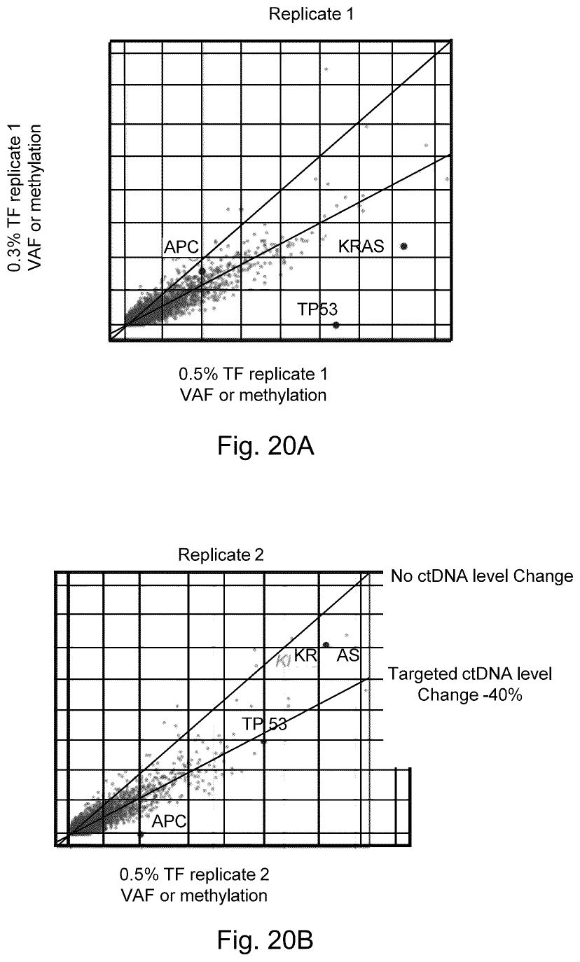

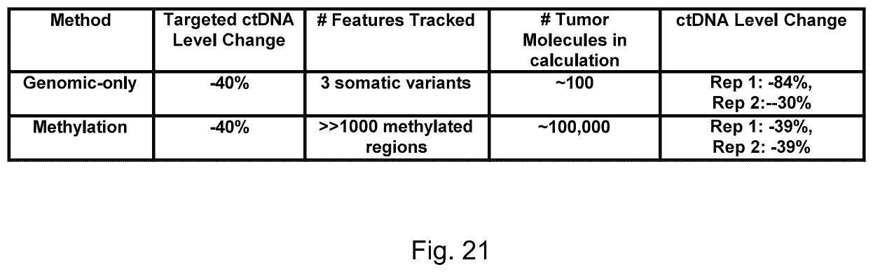

The robustness of epiMAF at low cTF is attributed to the high number of “evaluable” regions in the panel. Specifically, in two technical replicates of a clinical colorectal cancer (CRC) sample with titration levels at 0.5% and 0.3% cTF, epiMAF was estimated based on thousands of regions, whereas genomic cTF can only be estimated from three detectable somatic mutations. FIG. 20A is a graphical representation of methylation signals and somatic mutations for a first replicate of clinical titrations. FIG. 20B is a graphical representation of methylation signals and somatic mutations for a second replicate of clinical titrations. FIG. 21 is a table indicating ctDNA level changes for the first replicate and the second replicate calculated using a genomic-only method and a methylation method.

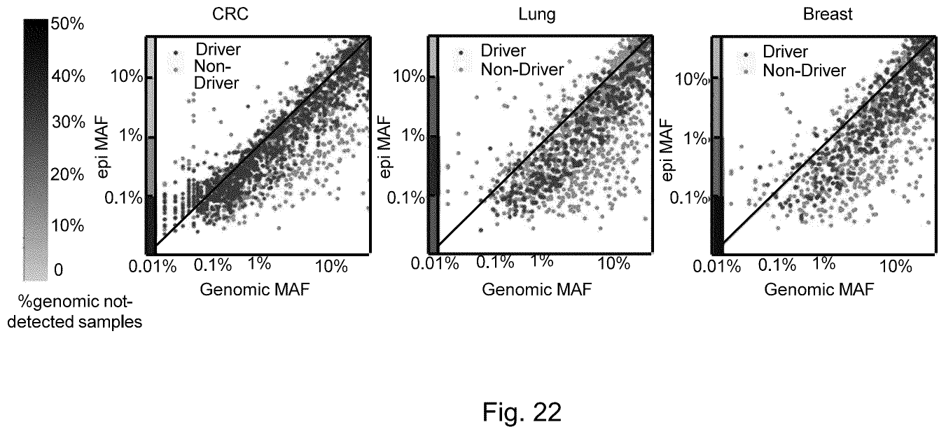

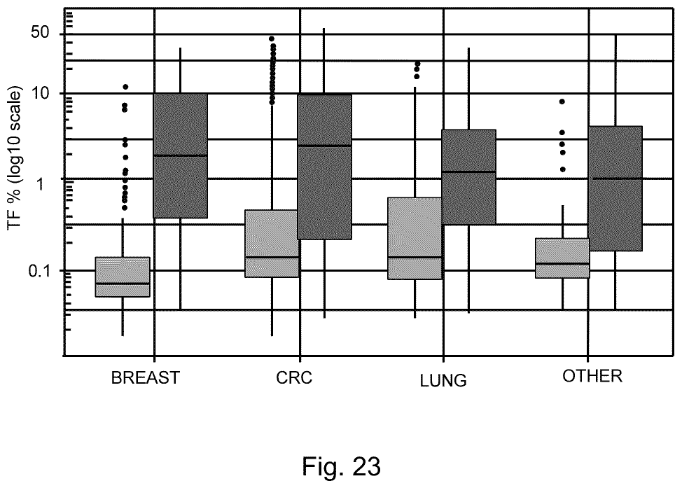

In a cohort of 5,045 clinical samples (CRC, lung, and breast cancer patients, (N=522, 909, 696 and 784 for stage I to IV, together with 2,656 of unknown stage), 64% had somatic mutations, and 90% showed evidence of the ctDNA presence based on methylation analysis. Notably, the epiMAF method produces highly consistent estimates with somatic MAFs for patients with “driver” somatic mutations, which are likely to be a more accurate representation of the true cTF. FIG. 22 is a graphical representation of epigenomic vs genomic cTF on clinical samples (one point for one sample). FIG. 23 is a graphical representation of the epiMAF distribution in early and late stage cancer patients for breast cancer, colorectal cancer, lung cancer, and a group of other cancers.

Upon analyzing 231 additional samples from various cancer types, the Inventors found that the majority of samples lacking detectable somatic mutations had tumor fractions below 0.1% (as shown in the side bars of FIG. 22). Patients with early-stage cancer are more prone to have such low tumor fractions where the current somatic mutation-based methods are unable to detect or quantify the mutations as indicated in FIG. 23.

Methylome sequencing enables accurate quantification of ctDNA level with a liquid-only approach, offering easy-to-access longitudinal ctDNA monitoring. Previous studies show that 30-50% patients with stage I-III cancer, and 15-20% patients with stage IV cancer, lack detectable somatic mutations. The methodologies described herein accurately detect and quantify cTF in these patients, improving patient evaluations and disease management.

### Example 5—MBD Partitioning Detection Scheme

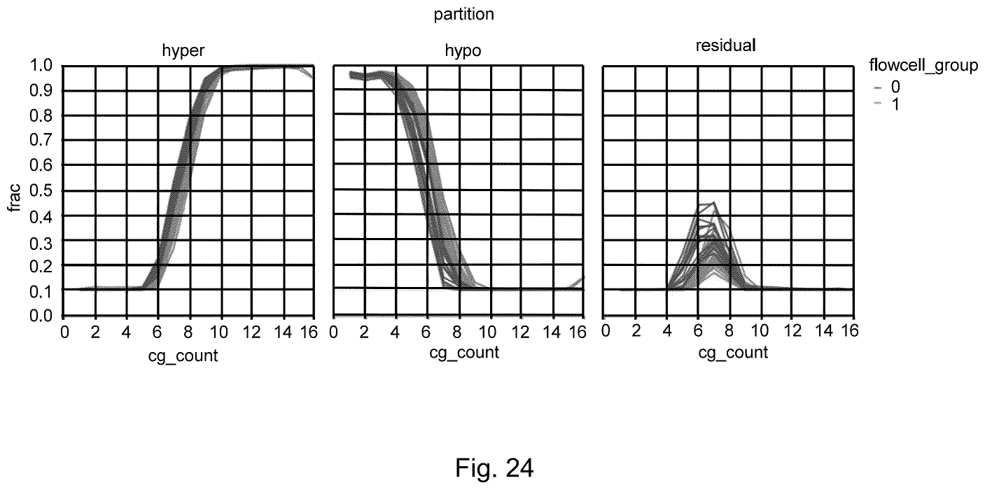

A number of subjects provided samples that were analyzed according to a cell-free DNA assay. Information derived from the samples by treating the samples with a number of solutions including MBD to separate molecules of the samples into a first partition, a second partition, and a third partition with each partition representing a different amount of methylated cytosines in genomic regions having an amount of CG content. The molecules having a given number of methylated CpGs were determined for each partition. FIG. 24 is a graphic showing a probability distribution indicating the number of methylated cytosines included in the three partitions.

For each sample, an MBD partitioning profile was calculated Q was determined according:

$$Q = [ \begin{matrix}{q}_{6} \\ … \\ {q}_{c} \\ … \\ {q}_{12}\end{matrix} ]$$

$${q}_{c} = {log}_{10} \frac{m}{M}$$

where where c=6, 7 . . . 12.
The qc values are standardized to q′_{c} based on training data. In these cases, mc is the number of molecules included in a hyper/residual partition with c CpGs from positive control regions and M is the total hyper/residual molecules with 12-30 CpGs from positive control regions and M=Σ_{c=12}³⁰m_{c}.

The region scores at region i (rᵢ) were normalized to determine normalized region scores r′ᵢ where

$${r}_{i}^{′} = {r}_{i} - {\sum }_{c = 6}^{12} ( {b}_{ic} , X {q}_{c}^{′} ) .$$

Additionally, the term b_{i,c} was estimated by fitting a linear regression model for individual classification regions using

$${r}_{i , s} = \frac{1}{S} X {\sum }_{s} {r}_{i , s} + {\sum }_{c = 6}^{12} ( {b}_{i , c} , X {q}_{c , s}^{′} )$$

$$\frac{1}{S} X {\sum }_{s} {r}_{i , s}$$

where is the mean score of region i.

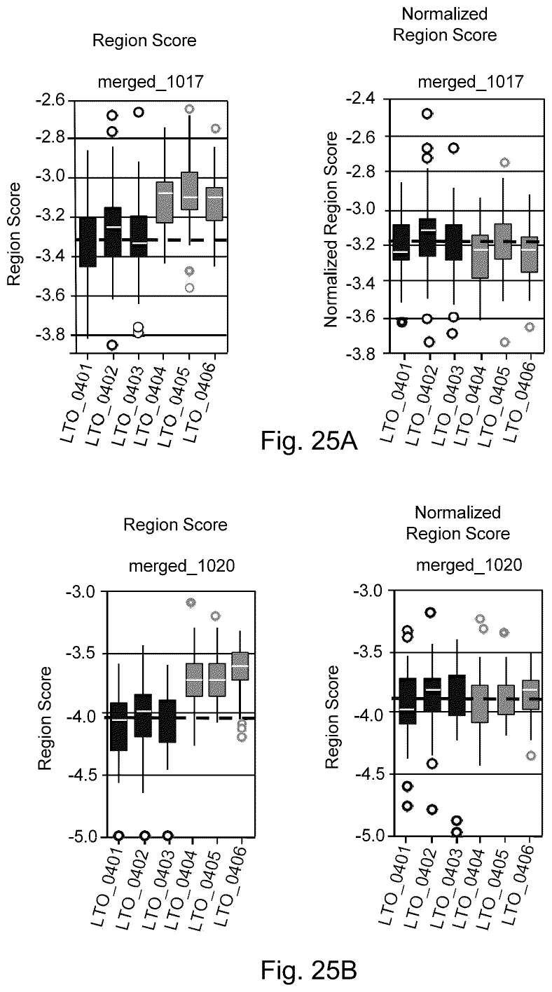

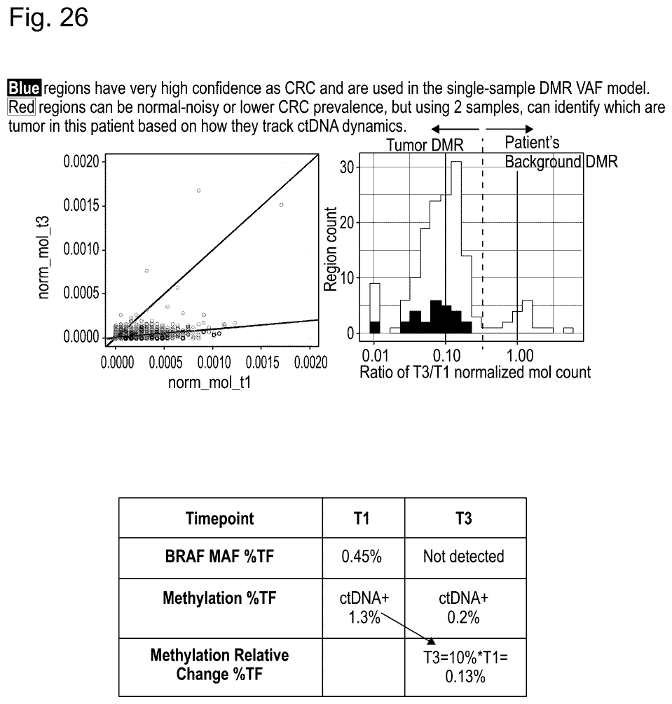

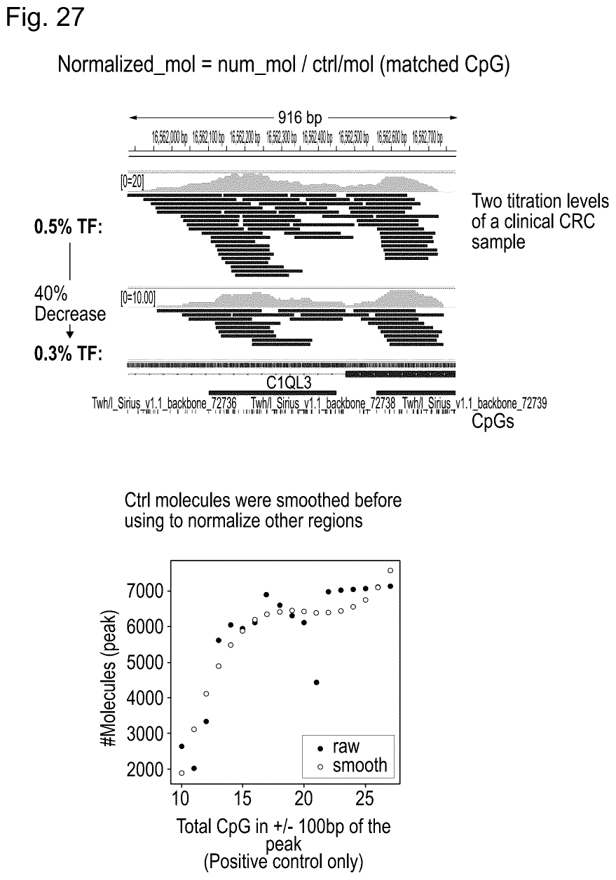

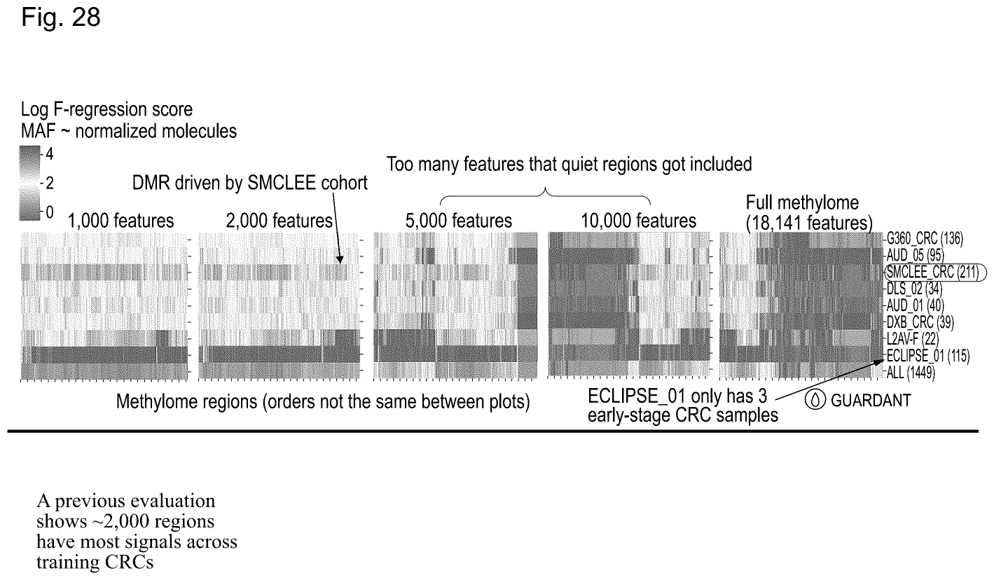

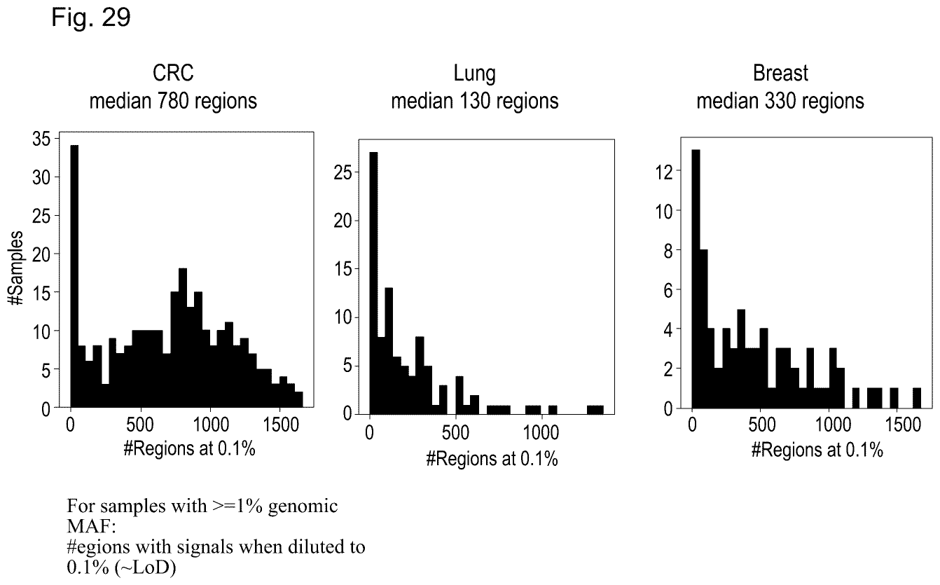

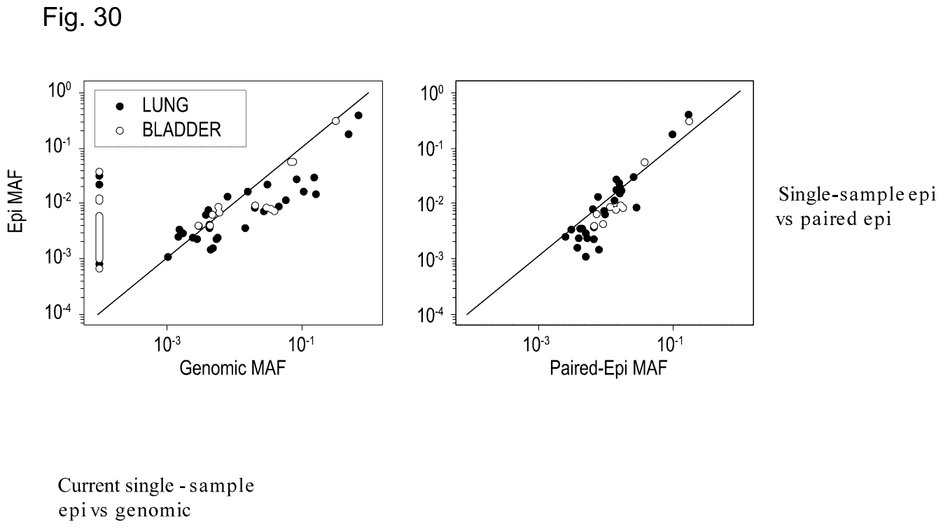

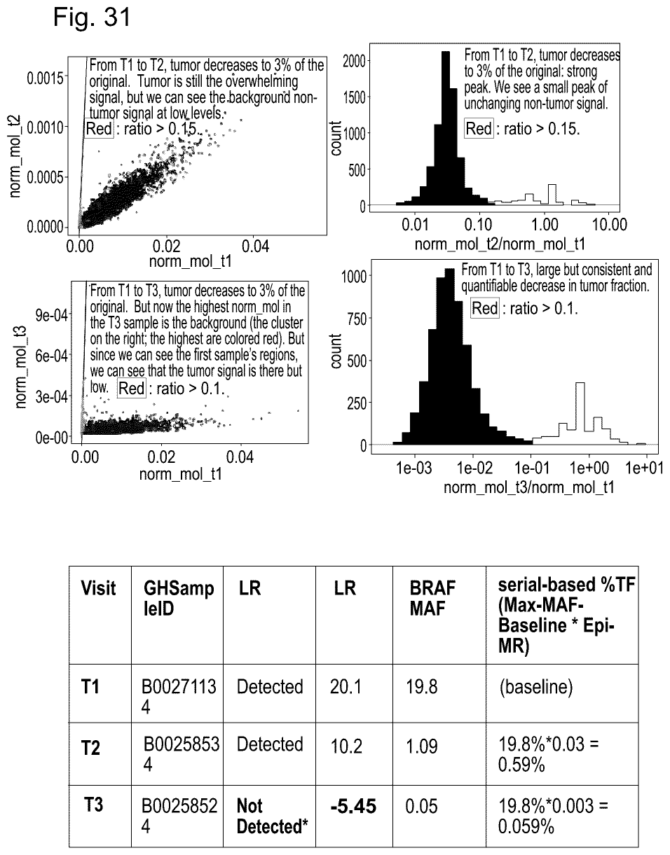

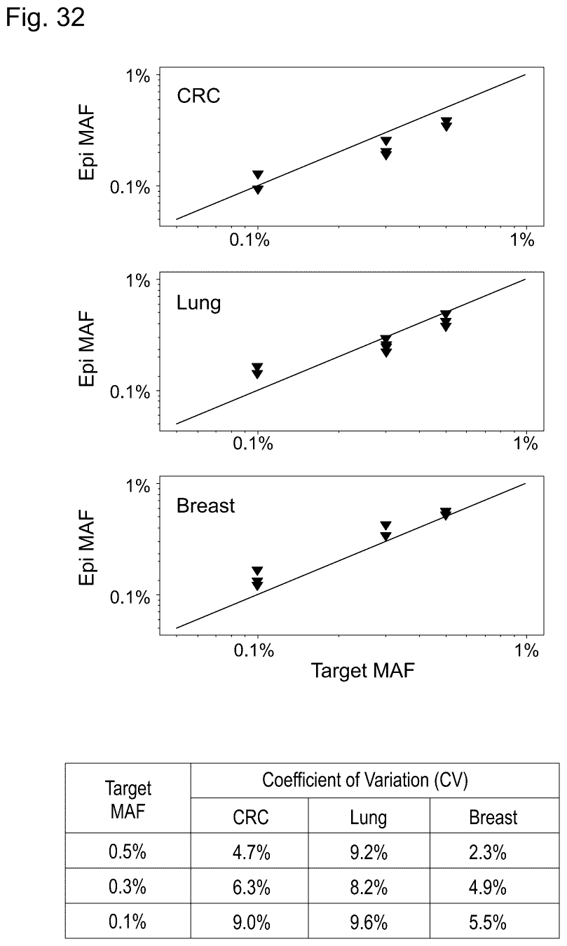

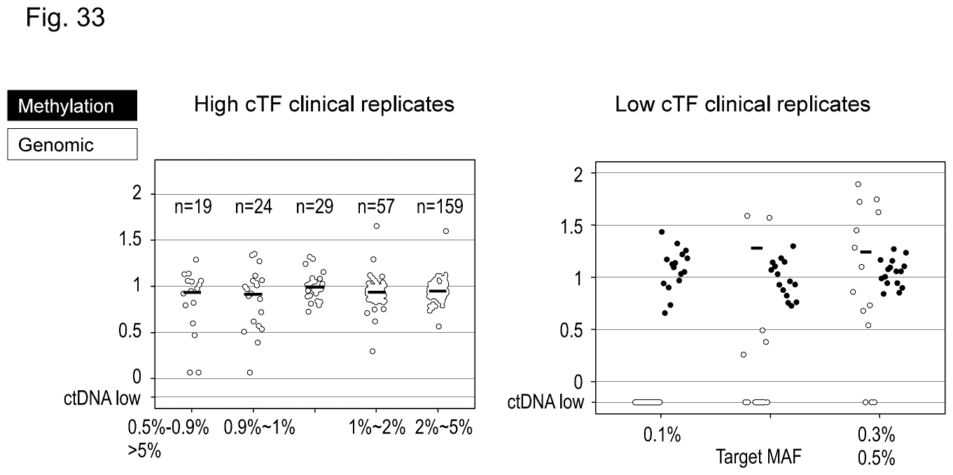

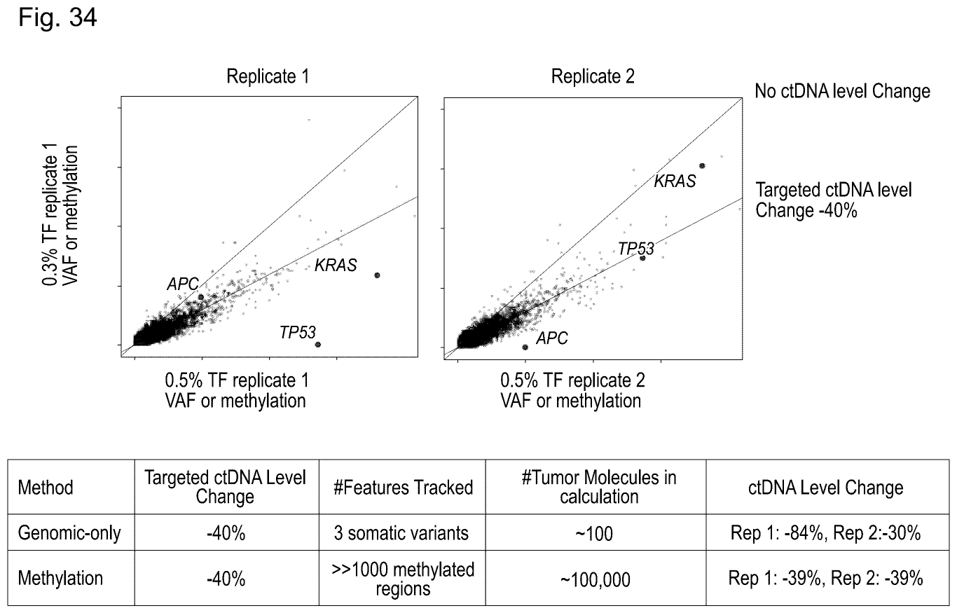

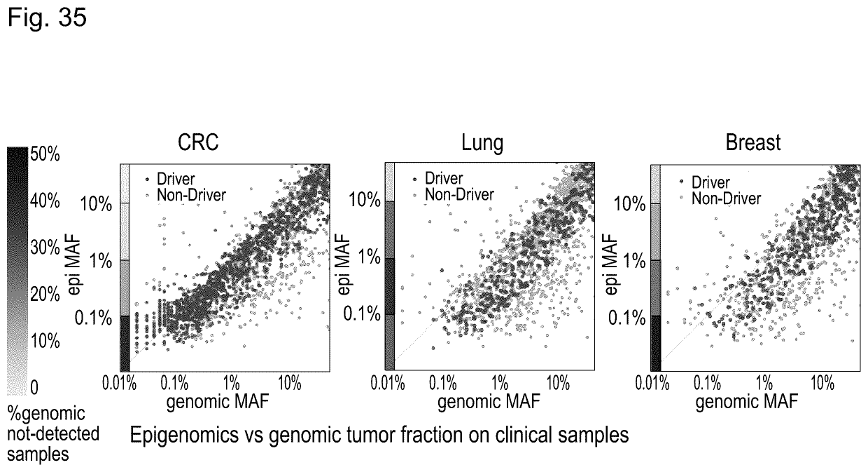

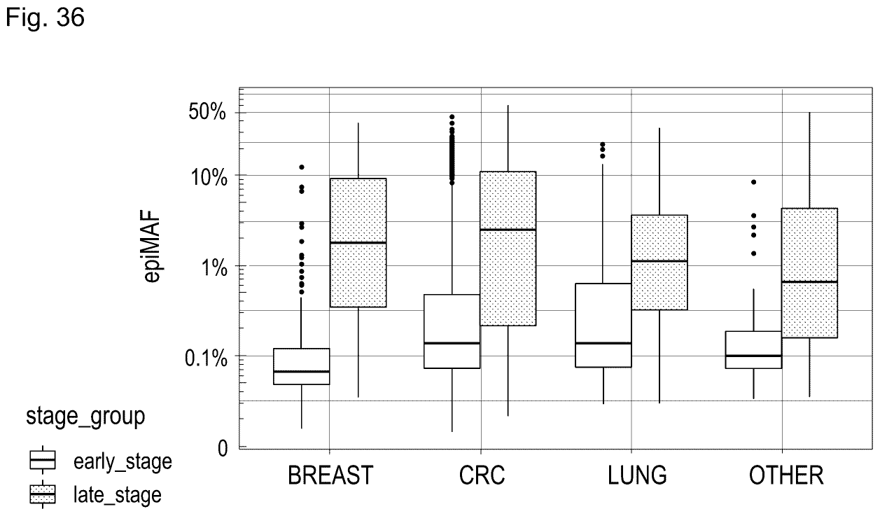

FIG. 25A includes a graphic showing changes to metrics for a first classification region for a first group of samples treated with MBD using a first set of reagents and a second group of samples treated with MBD using a second set of reagents. Additionally, FIG. 25B includes a graphic showing changes to metrics for a second classification region for a first classification region for a first group of samples treated with MBD using a first set of reagents and a second group of samples treated with MBD using a second set of reagents.

### Example 6—Method Summary

| | |
|---|---|
| DMR | Select from ~2000 cancertype-specific cancer-prevalent- |
| features | normal-quiet regions: |
| | Normalized by positive control |
| | Molecule count at in-sample peak |
| | No null subtraction |
| DMR | N regions = 300 top |
| aggregation | Outlier removal = 2% of ~2000 regions = ~40 regions |
| | Scaling factor = 0.76 |

### Example 7—Additional Method Summary for Samples Such as Lung

| Method summary: | Application to Samples such as Lung |
|---|---|
| DMR features | Select from ~2000 cancertype-specific cancer- |
| | prevalent-normal-quiet regions: |
| | Normalized by positive control |
| | Noise reduction: |
| | Molecule count at fixed peak |
| | Subtract null |
| DMR | N regions = Reduce to 100 top |
| aggregation | Outlier removal using MAD >5 |
| | Scaling factor = Update to 1.0 |

### Example 8—Method Summary

Generally, normalization is performed by comparing number of molecules against a control for match CpG. Thereafter, one can utilize a set of differentially methylated regions (DMRs) that, including from a database including information using a trained dataset. Of interest, is excluding regions with too frequent signals in normal, as in a single-sample setting, it's impossible to distinguish normal/cancer signals. Additionally, of interest is selecting high-prevalence cancer-specific regions, as selecting low-prevalence regions (more signals) introduces additional noise.

Thereafter, one selects top regions to estimate epi MAF. Better to define the possibly methylated regions in each sample as prevalence analysis indicates not all regions are methylated in all samples. epiTF can be calculated when methylation is detected, with a Limit of Quantification (LoQ), the TF where variation <=defined threshold, focus on >=0.1

Additionally, determination of sample TF % from sample's top regions can include using a decision tree. Among the selected regions, one select the sample's 100 regions with highest methylation “VAF” wherein % TF=Average methylation “VAF” of these region. In some instances it may be beneficial to take 5%˜95% quantile to avoid outliers.

### Example 9—Problem of TF Quantification, Differentially Methylated Regions (DMRs)

Problem of TF quantification, including difficulties in identifying which DMR are present in cancer, the fraction of signal in DMR from cancer and clonality of the cancer DMR and relationship to tumor fraction.

Straight counting tumor derived molecules is not trivial. On cancer hyper DMRs, tumor derived molecules are hypermethylated molecules. However, different subsets of DMRs may have signal in different cancer types, and different samples within cancer types.

This supports a model that has the capability to identify patient-specific clonal DMRs. However, limits include counting noise: Low number of signal molecules on the tumor specific DMRs makes detecting low tumor fraction challenging and biological noise: hypermethylated molecules from normal cells can be mistreated as tumor signal.

### Example 10—Normalization

One may utilize a trained dataset of colorectal cancer, with the described methods and compositions measuring up to 18,141 features. It can be seen that 2,000 regions have most signals across samples, as expanding beyond 2,000 does not continue identifying differential methylated regions, with further drawbacks of including quite regions.

Thereafter a molecule count, including normalized molecule count by comparison to control molecule counts of matched CpGs.

### Example 11—Methods

The epigenomics cTF (or methyl cTF) of a single sample is estimated from methylation signals across targeted regions of the Combined genomic and/or epigenomic detection assay described herein methylation panel, calibrated using our internal training data that has clinical blood draw samples of over 5,000 individuals, including cancer-free donors and patients with mixed cancer types. Somatic mutations were also detected through the a combined genomic and/or epigenomic panel. The genomic cTF (or somatic cTF) is defined as the highest VAF of detected somatic mutations.

LoD was determined as the minimum cTF level at which >=95% of replicates exhibited methylation signals derived from tumors. LoQ was defined as the minimum cTF level at which the coefficient of variation (CV) across replicates was less than 30%. The accuracy of methylation-based cTF was compared to cTFs derived from the maximum MAF of somatic mutations on 5,045 clinical samples of cancer patients.

### Example 12—Results

One colorectal cancer sample, one breast cancer sample, one lung cancer sample, and one cell line sample were titrated into cancer-free backgrounds at target levels ranging from 0.1% to 0.5% MAF. The methylation LoD, which was defined as the lowest concentration of tumor-derived DNA detectable with >95% accuracy, was estimated to be approximately 0.05% at the input level of 5-30 ng. (See poster #6601 “Analytical validation of a robust integrated genomic and epigenomic liquid biopsy for biomarker discovery, therapy selection and response monitoring”).

The methyl cTF of clinical samples exhibit a high degree of consistency with underlying titration levels and maintain a strong linearity between different titration levels, as indicated by a Pearson-r of greater than 0.9 and a linearity error less than 5% (FIG. 1).

The quantitative precision of the methyl cTF is capable of reaching an LoQ of less than 0.1% in CRC, lung and breast clinical samples (Table 1). The genomic cTF is robust for replicates within the same cTF levels, particularly at cTF levels of 1% or higher (FIG. 2, left panel). However, at lower titration levels, the methyl cTF is more stable. The epigenomic cTF can maintain a 100% evaluation rate and has a LoQ down to 0.1% cTF (FIG. 2, right panel).

The robustness of methyl cTF is attributed to the high number of “evaluable” regions in the panel. Specifically, in two technical replicates of a clinical colorectal cancer (CRC) sample with titration levels at 0.5% and 0.3% cTF, the methyl cTF was estimated based on thousands of regions, whereas the genomic cTF can only be estimated from three detectable somatic mutations (FIG. 3 and Table 2).

### Example 13—Add'l Results

In a cohort of 5,045 clinical samples (CRC, lung, and breast cancer patients, (N=522, 909, 696 and 784 for stage I to IV, together with 2,656 of unknown stage), 64% had somatic mutations, and 90% showed evidence of the ctDNA presence based on methylation analysis. Notably, in FIG. 4 with Pearson-r(driver) >0.9, methyl cTF is highly consistent with somatic MAFs from “driver” mutations, which may be a more accurate representation of cTF than “non-driver” mutations.

Upon analyzing 231 additional samples from various cancer types, the Inventors found that the majority of samples lacking detectable somatic mutations had epigenomics tumor fractions below 0.1% (side bars of FIG. 4). Samples from early-stage cancer has a significantly lower cTF than late-stage cancer (FIG. 5, paired t-test p<0.01). In these early-stage samples, somatic mutation-based methods are unable to detect evidence of ctDNA.

What is claimed is:

1. A method comprising:
obtaining training sequence data including training sequencing reads derived from a plurality of samples of a plurality of subjects, individual training sequencing reads including a nucleotide sequence corresponding to a fragment of a nucleic acid included in a sample of the plurality of samples and an amount of methylated cytosines included in regions of the nucleotide sequence having cytosine-guanine content;
analyzing the training sequencing reads to determine a first quantitative measure derived from the training sequencing reads that corresponds to individual classification regions of a plurality of classification regions, at least a portion of the individual classification regions of the plurality of classification regions corresponding to genomic regions of a reference genome that have an amount of methylated cytosines in subjects in which cancer is detected;
analyzing the training sequencing reads to determine a second quantitative measure derived from the training sequencing reads that correspond to a plurality of control regions, individual control regions of the plurality of control regions corresponding to additional genomic regions of the reference genome that have an amount of methylated cytosines in which cancer is not detected;
determining a metric for the individual classification regions of the plurality of classification regions based on the first quantitative measure for the individual classification regions and the second quantitative measure for the plurality of control regions;
generating training data comprising the metric for the individual classification regions of the plurality of classification regions for the training sequence reads from samples of training subjects;
implementing one or more machine learning algorithms to generate a model, the model including weights for individual classification regions of the plurality of classification regions and at least a portion of the weights of the individual classification regions being different from one another.

2. The method of claim 1, comprising:
obtaining testing sequence data from an additional subject that is not included in the plurality of subjects, the testing sequence data including testing sequencing reads derived from a sample of the additional subject, individual testing sequencing reads including a nucleotide sequence corresponding to a fragment of a nucleic acid included in the additional sample and individual testing sequencing reads corresponding to molecules having an amount of methylated cytosines included in regions of the nucleotide sequence having cytosine-guanine content; and
determining, using the model and the additional sequence data, a measurement of tumor fraction in the additional subject.

3. The method of claim 1, comprising selecting a sub-set of the plurality of classification regions.

4. The method of claim 3, wherein the sub-set of the plurality of classification regions comprise one or more cancer-specific regions.

5. The method of claim 1, wherein the metric for the individual classification regions of the plurality of classification regions based on the first quantitative measure for the individual classification regions and the second quantitative measure for the plurality of control regions comprises a sub-set of the plurality of classification regions.

6. The method of claim 2, comprising:
analyzing the testing sequencing reads to determine a first quantitative measure derived from the testing sequencing reads that correspond to the individual classification regions of the plurality of classification regions;
analyzing the testing sequencing reads to determine a second quantitative measure derived from the testing sequencing reads that correspond to the individual control regions of the plurality of control regions;
normalizing the second quantitative measure based on the corresponding individual control regions of the plurality of control regions;
determining the metric for the individual classification regions based on the first quantitative measure for the individual classification regions and the normalized second quantitative measure for the plurality of control regions; and
applying a machine learning algorithm to the metrics for the individual classification regions to determine a measurement of tumor fraction in the additional subject.

7. The method of claim 1-6, wherein:
the one or more machine learning algorithms comprise one or more classification algorithms.

8. The method of claim 1-7, wherein the one or more machine learning algorithms comprises one or more regression algorithms.

9. The method of claim 8, wherein applying a machine learning algorithm to the metrics for the individual classification regions to determine a measurement of tumor fraction in the additional subject comprises selecting a sub-set of the plurality of classification regions

10. The method of claim 1-9, wherein:
the training data comprises the individual measures of tumor fraction for the individual samples of the plurality of samples; and
the model is generated based on the individual measures of tumor fraction for the individual samples of the plurality of samples.

11. The method of claim 1-10, wherein the metric for the individual classification regions is determined based on a scaling factor and/or an error correction factor.

12. The method of claim 1-11, wherein the plurality of classification regions individually correspond to genomic regions in which a methylation rate of cytosines in the genomic regions of nucleic acids derived from cells obtained from subjects in which cancer is present is different from a methylation rate of cytosines in the genomic regions of nucleic acids derived from cells obtained from subjects in which cancer is not present.

13. The method of claim 1-12, wherein the plurality of classification regions correspond to a first plurality of classification regions for a first cancer type and the model can be generated for a second cancer type based on a second plurality of classification regions that are different from the first plurality of classification regions.

14. A method comprising:
obtaining sequencing reads derived from a sample obtained from a subject, individual sequencing reads including a nucleotide sequence corresponding to a fragment of a nucleic acid included in the sample and corresponding to molecules having a threshold amount of methylated cytosines included in regions of the nucleotide sequence having cytosine-guanine content;
determining, by the computing system, a first quantitative measure derived from the sequencing reads that corresponds to individual classification regions of a plurality of classification regions, at least a portion of the individual classification regions of the plurality of classification regions corresponding to genomic regions of a reference genome with amount of methylated cytosines in subjects in which cancer is detected;
analyzing, by the computing system, the sequencing reads to determine a second quantitative measure derived from the sequencing reads that correspond to a plurality of control regions, individual control regions of the plurality of control regions corresponding to additional genomic regions of the reference genome that have cytosine-guanine content and an amount of methylated cytosines in additional subjects in which cancer is not detected;
determining, by the computing system, a plurality of metrics with individual metrics of the plurality of metrics corresponding to individual classification regions of the plurality of classification regions based on the first quantitative measure for the individual classification regions and the second quantitative measure for the plurality of control regions; and
determining a measurement of tumor fraction in the additional subject.

15. The method of claim 14, comprising selecting a sub-set of the plurality of classification regions.

16. The method of claim 15, wherein the sub-set of the plurality of classification regions comprise one or more cancer-specific regions.

17. The method of claim 14, wherein the metric for the individual classification regions of the plurality of classification regions based on the first quantitative measure for the individual classification regions and the second quantitative measure for the plurality of control regions comprises a sub-set of the plurality of classification regions.

18. The method of claim 14-17, comprising:
determining an order of the values of the plurality of metrics; and
determining a subset of classification regions from among the plurality of classification regions based on the order;
wherein a portion of the plurality of metrics that correspond to the subset of the classification regions is used to determine a measurement of tumor fraction in the additional subject.

19. The method of claim 14-18, wherein the determining a measurement of tumor fraction in the additional subject comprises applying a scaling factor.

20. The method of claim 14-19, wherein the determined measurement of tumor fraction corresponds to an indication of cancer status in the subject.

21. The method of claim 14-20, wherein determining a measurement of tumor fraction in the subject comprises,
applying a model generated from training data.

22. The method of claim 14-21, wherein the model generated from training data comprises:
obtaining training sequence data including training sequencing reads derived from a plurality of samples of a plurality of subjects, individual training sequencing reads including a nucleotide sequence corresponding to a fragment of a nucleic acid included in a sample of the plurality of samples and an amount of methylated cytosines included in regions of the nucleotide sequence having cytosine-guanine content;
analyzing the training sequencing reads to determine a first quantitative measure derived from the training sequencing reads that corresponds to individual classification regions of a plurality of classification regions, at least a portion of the individual classification regions of the plurality of classification regions corresponding to genomic regions of a reference genome that have a threshold amount of methylated cytosines in subjects in which cancer is detected and that have at least the threshold cytosine-guanine content;
analyzing the training sequencing reads to determine a second quantitative measure derived from the training sequencing reads that correspond to a plurality of control regions, individual control regions of the plurality of control regions corresponding to additional genomic regions of the reference genome that have at least the threshold cytosine-guanine content and that have a threshold amount of methylated cytosines in which cancer is not detected;
determining a metric for the individual classification regions of the plurality of classification regions based on the first quantitative measure for the individual classification regions and the second quantitative measure for the plurality of control regions;
generating training data comprising the metric for the individual classification regions of the plurality of classification regions for the training sequence reads from samples of training subjects;
implementing one or more machine learning algorithms to generate the model.

23. A method comprising:
obtaining testing sequence data from a subject, the testing sequence data including testing sequencing reads derived from a sample of the subject, individual testing sequencing reads including a nucleotide sequence corresponding to a fragment of a nucleic acid included in the additional sample and individual testing sequencing reads corresponding to molecules having an amount of methylated cytosines included in regions of the nucleotide sequence;
analyzing the testing sequencing reads to determine a first quantitative measure derived from the testing sequencing reads that correspond to individual classification regions of a plurality of classification regions at least a portion of the individual classification regions of the plurality of classification regions corresponding to genomic regions of a reference genome that have an amount of methylated cytosines in subjects in which cancer is detected;
analyzing the testing sequencing reads to determine a second quantitative measure derived from the testing sequencing reads that correspond to individual control regions a plurality of control regions, individual control regions of the plurality of control regions corresponding to additional genomic regions of the reference genome that have an amount of methylated cytosines in additional subjects in which cancer is not detected;
determining a metric for the individual classification regions based on the first quantitative measure for the individual classification regions and the second quantitative measure for the plurality of control regions; and
generating training data that includes the metric for the individual classification regions of the plurality of classification regions for the training sequence reads from samples of training subjects;
implementing one or more machine learning algorithms to generate a model, the model including weights for individual classification regions of the plurality of classification regions and at least a portion of the weights of the individual classification regions being different from one another to determine a measurement of tumor fraction in the subject.

24. The method of claim 23, comprising:
obtaining training sequence data including training sequencing reads derived from a plurality of samples of a plurality of training subjects, individual training sequencing reads including a nucleotide sequence corresponding to a fragment of a nucleic acid included in a sample of the plurality of samples and individual training sequencing reads corresponding to molecules having a threshold amount of methylated cytosines included in regions of the nucleotide sequence having at least a threshold cytosine-guanine content;
analyzing the training sequencing reads to determine an additional first quantitative measure derived from the training sequencing reads that corresponds to individual classification regions of the plurality of classification regions;
analyzing the training sequencing reads to determine an additional second quantitative measure derived from the training sequencing reads that correspond to a plurality of control regions;
determining an additional metric for the individual classification regions of the plurality of classification regions based on the additional first quantitative measure for the individual classification regions and the additional second quantitative measure for the plurality of control regions;
generating training data comprising the additional metric for the individual classification regions of the plurality of classification regions for the training sequence reads from samples of the plurality of training subjects;
implementing using the training data, one or more machine learning algorithms to generate the model to determine the indications of cancer status in subjects based on amounts of methylated cytosines in at least a portion of the plurality of classification regions.

25. The method of claim 23-24, wherein:
the one or more machine learning algorithms include one or more classification algorithms.

26. The method of claim 23-25, wherein the one or more machine learning algorithms comprising one or more regression algorithms; and
the indication corresponds to an estimate of tumor fraction of the sample.

27. The method of claim 23-26, wherein the training sequencing reads comprise a first portion of the training sequence data and additional training sequencing reads comprise a second portion of the training sequence data, wherein the additional training sequencing reads are different from the training sequencing reads; and
the method comprising:
analyzing at least one of the first portion of the training sequence data or the second portion of the training sequence data to determine an individual frequency of a plurality of variants present in an individual sample of the plurality of samples;
determining for the individual sample, a variant of the plurality of variants having a maximum frequency that corresponds to the individual frequency having a greatest value among individual frequencies derived from an individual sample; and
determining individual measures of tumor fraction for an individual sample based on the greatest value of the individual frequencies derived from the individual sample.

28. The method of claim 23-27, wherein:
the training data includes the individual measures of tumor fraction for the individual samples of the plurality of samples; and
the model is generated based on the individual measures of tumor fraction for the individual samples of the plurality of samples.

29. The method of any one of claims 23-28, wherein the sample of the subject and the plurality of samples of the plurality of training subjects include cell free nucleic acids.

30. A method comprising:
obtaining sequencing reads derived from one or more samples obtained from a subject, individual sequencing reads including a nucleotide sequence corresponding to a fragment of a nucleic acid included in the sample and corresponding to molecules having a threshold amount of methylated cytosines included in regions of the nucleotide sequence having at least a threshold cytosine-guanine content;
determining a first quantitative measure derived from the sequencing reads that corresponds to individual classification regions of a plurality of classification regions, at least a portion of the individual classification regions of the plurality of classification regions corresponding to genomic regions of a reference genome that an amount of methylated cytosines in subjects in which cancer is detected and that have at least the threshold cytosine-guanine content;
analyzing the sequencing reads to determine a second quantitative measure derived from the sequencing reads that correspond to a plurality of control regions, individual control regions of the plurality of control regions corresponding to additional genomic regions of the reference genome that have at least the threshold cytosine-guanine content and that have an amount of methylated cytosines in additional subjects in which cancer is not detected;
determining a plurality of metrics with individual metrics of the plurality of metrics corresponding to individual classification regions of the plurality of classification regions based on the first quantitative measure for the individual classification regions and the second quantitative measure for the plurality of control regions; and
determining an indication of cancer status in the subject based on at least a portion of the plurality of metrics.

31. The method of claim 30, comprising selecting a sub-set of the plurality of classification regions.

32. The method of claim 30-31, wherein the sub-set of the plurality of classification regions comprise one or more cancer-specific regions.

33. The method of claim 30-32, wherein the metric for the individual classification regions of the plurality of classification regions based on the first quantitative measure for the individual classification regions and the second quantitative measure for the plurality of control regions comprises a sub-set of the plurality of classification regions.

34. The method of claim 30-33, comprising at least two samples obtained from a subject

35. The method of claim 30-34, wherein determining a metric for the individual classification regions of the plurality of classification regions based on the first quantitative measure for the individual classification regions and the second quantitative measure for the plurality of control regions comprises:
selecting a sub-set of the plurality of classification regions based on
a regression algorithm based on the first quantitative measure for the individual classification regions and the second quantitative measure for the plurality of control regions.

36. The method of claim 30-35, wherein the first quantitative measure is normalized based on second quantitative measure.

37. The method of any preceding claim, wherein the plurality of samples and the additional sample comprise cell free nucleic acids.

38. The method of any preceding claim, comprising:
combining a plurality of nucleic acids derived from at least one of blood or tissue of a subject with a solution including an amount of methyl binding domain (MBD) proteins to produce a nucleic acid-MBD protein solution; and
performing a plurality of washes of the nucleic acid-MBD protein solution with a salt solution to produce a number of nucleic acid fractions, individual nucleic acid fractions having a threshold number of methylated cytosines in regions of the plurality of nucleic acids having at least the threshold cytosine-guanine content.

39. The method of any preceding claim, wherein a wash of the plurality of washes is performed with a solution having a concentration of sodium chloride (NaCl) and produces a nucleic acid fraction of the number of nucleic acid fractions having a range of binding strengths to MBD proteins.

40. The method of any preceding claim, comprising:
determining that a first nucleic acid fraction is associated with a first partition of a plurality of partitions of nucleic acids, the first partition corresponding to a first range of binding strengths to MBD proteins;
attaching a first molecular barcode to nucleic acids of the first nucleic acid fraction, the first molecular barcode being included in a first set of molecular barcodes associated with the first partition;
determining that a second nucleic acid fraction is associated with a second partition of the plurality of partitions of nucleic acids, the second partition corresponding to a second range of binding energies to MBD proteins different from the first range of binding strengths to MBD proteins; and
attaching a second molecular barcode to nucleic acids of the second nucleic acid fraction, the second molecular barcode being included in a second set of molecular barcodes associated with the second partition.

41. The method of any preceding claim, comprising:
combining at least a portion of the number of nucleic acid fractions with an amount of restriction enzyme that cleaves molecules with one or more unmethylated cytosines to produce at least a portion of the plurality of samples used to produce the sequencing reads, wherein the threshold amount of methylated cytosines corresponds to a minimum frequency of methylated cytosines within a region having at least the threshold cytosine-guanine content.

42. The method of any preceding claim, comprising:
combining at least a portion of the number of nucleic acid fractions with an amount of a restriction enzyme that cleaves molecules with one or more methylated cytosines to produce at least a portion of the plurality of samples used to produce the sequencing reads, wherein the threshold amount of unmethylated cytosines corresponds to a maximum frequency of methylated cytosines that are not cleaved within a region having at least the threshold cytosine-guanine content.

43. The method of any preceding claim, wherein a limit of detection for the model to determine tumor fraction of samples is no greater than 0.05%. 0.05%.

44. A system comprising instructions for processing the methods of any preceding claim.

45. A computer readable medium comprising instructions for processing the methods of any preceding claim.
